(19) United States
(12) Patent Application Publication
(10) Pub. No.: US 2008/0039461 A1
(43) Pub. Date: Feb. 14, 2008

(54) Treatment of pain by inhibition of p38 map kinase

(76) Inventors: Andrew A. Protter, Palo Alto, CA (US); Camilla Svensson, San Diego, CA (US); Tony Yaksh, San Diego, CA (US); Barbara Cordell, Palo Alto, CA (US); Sundeep Dugar, San Jose, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/648,226

(22) Filed: Dec. 28, 2006

Related U.S. Application Data
(63) Continuation of application No. 11/429,900, filed on May 8, 2006, now abandoned.
Application No. 11/429,900 is a continuation of application No. 11/208,351, filed on Aug. 19, 2005, now abandoned.
Application No. 11/208,351 is a continuation-in-part of application No. 10/655,745, filed on Sep. 5, 2003, now abandoned.
(60) Provisional application No. 60/408,610, filed on Sep. 5, 2002.

(51) Int. Cl. A61K 31/535; A61K 31/415; A61K 31/445; A61P 25/00; A61K 31/505

(52) U.S. Cl. 514/235.2; 435/29; 514/235.8; 514/236.5; 514/274; 514/323; 514/407

(57) ABSTRACT

The present invention relates to methods for the prevention or treatment of pain by the inhibition of p38 MAP kinase.

### RELATED APPLICATIONS

This application is a continuation of U.S. patent application Ser. No. 11/429,900, filed on May 8, 2006, which is a continuation of U.S. patent application Ser. No. 11/208,351, filed on Aug. 19, 2005, which is a continuation-in-part of U.S. patent application Ser. No. 10/655,745, filed on Sep. 5, 2003, which claims the benefit of priority of U.S. Provisional Patent Application No. 60/408,610, filed on Sep. 5, 2002, all disclosures of which are hereby incorporated by reference in their entirety.

### STATEMENT REGARDING FEDERALLY SPONSORED RESEARCH OR DEVELOPMENT

This invention is supported in part by Grant No. NS16541 of the National Institutes of Health. The United States government may have certain rights in this invention.

### BACKGROUND OF THE INVENTION

Pain continues to present a significant obstacle for medical management and treatment, and accompanies physical injury, and surgery, as well as many chronic disease states. In 1998 alone, 38.7 million and 63.8 million were treated in the United States for chronic and acute pain, respectively. However, most pain-ablating treatments have significant, and sometimes debilitating, side effects. For example, the commonly used nonsteriodal anti-inflammatory drugs have gastrointestinal side effects, while narcotics administered for more severe pain are limited by addiction potential, tolerance development, constipation, gastrointestinal distress, and respiratory depression. Accordingly, there is a great need to develop new methods of treatment that will avoid such deleterious side effects, while still effectively ameliorating pain.

Pain is elicited by a nociceptive event wherein environmental stimuli are converted into electrochemical and protein signals that are then transmitted from the periphery to the brain. Physiological pain is initiated by sensory nociceptor fibers innervating peripheral tissues following a noxious mechanical, chemical or thermal stimuli. The subsequent sensory response elicits the perception of pain through the activation of neurons in the spinal cord, which project to the cortex via a relay in the thalamus. This activation threshold of physiological pain can be lowered as a result of prior activation or from intense or sustained stimulation. Pathological pain, on the other hand, can be produced by innocuous stimuli not normally capable of inducing a pain state (allodynia) or by noxious stimuli that evoke a greater and more prolonged pain (hyperalgesia). Allodynia can result from two different conditions: increased responsiveness of spinal cord ‘pain’ transmission neurons (central sensitization) or lowered nociceptor activation thresholds (peripheral sensitization). With central sensitization, pain can be produced by activity in the primary sensory C fibers. Peripheral sensitization is produced when nociceptive A-δ fiber terminal become exposed to products of tissue damage and inflammation. The C fiber central sensitization and A-δ fiber peripheral sensitization processes can be analyzed separately in vivo using different behavioral models (reviewed by Yaksh, T., Trends in Pharm. Sci. (1999) 20: 329-337).

Inflammatory pain and neuropathic pain exemplify hyperalgesia, wherein tissue damage and inflammation initiate inflammatory pain. Such inflammatory pain results in pain hypersensitivity that generally returns to normal, but only if the induction process is controlled and is reversible. Otherwise, a chronic state of hyperalgesia ensues. Similarly, nervous system lesions or disease initiates neuropathic pain, which is a chronic state of hyperalgesia, that usually persists long after the initiating event has been resolved.

A wide variety of intracellular signaling molecules permit neurons and other cells to respond to environmental stimuli. MAP kinases transduce signals received from an extracellular stimulus to the nucleus, permitting the individual cell to respond to changes within its microenvironment.

p38 MAP kinase is a member of a family of signaling molecules known as the mitogen-activated protein kinase (MAP kinase) family. p38 MAP kinase is activated by a variety of cellular stressors, including ultraviolet radiation, osmotic shock, and inflammatory cytokines, such as IL-1 and TNF. Four isoforms of p38 have been identified and are designated as p38α, p38β, p38γ and p38δ. Jiang, Y., et al., J Biol Chem (1996) 271:17920-17926; Kumar, S., et al., Biochem Biophys Res Comm (1997) 235:533-538; Stein, B., et al., J Biol Chem (1997) 272:19509-19517; Li, Z., et al., Biochem Biophys Res Comm (1996) 228:334-340; Wang, X., et al., J Biol Chem (1997) 272:23668-23674. These isoforms differ in tissue expression patterns, substrate utilization, response to direct and indirect stimuli, and susceptibility to kinase inhibitors. For example, one study has demonstrated the activation of p38β MAP kinase results in myocyte hypertrophy, while the activation of p38α MAP kinase leads to myocyte apoptosis. Wang, Y., et al., J Biol Chem (1998) 273:2161-2168.

p38 MAP kinase activation is mediated in certain neuronal cells (retinal ganglion neurons) by increased glutamate through the NMDA glutamate receptors. NMDA receptors also mediate the fast excitatory transmission at synapses in the spinal cord and other regions of the central nervous system that are crucial in nociception, in particular, central sensitization (reviewed in Woolf & Salter, Science (2000) 288:1765-1768). Under physiologic conditions, p38 MAP kinase activation appears transient. Once activated, p38 mediates the induction of mRNA synthesis for a variety of inflammatory mediators, including IL-1β, TNF-α, IL-6, and COX-2.

### BRIEF SUMMARY OF THE INVENTION

The present invention relates to methods for the prevention or treatment of pain, by the inhibition of p38 MAP kinase.

In one aspect, the present invention provides a method to prevent or treat pain in a mammal in need thereof comprising administering an inhibitor of p38 kinase in a therapeutically effective amount to said mammal.

In another aspect, the present invention provides a method to prevent or treat pain in a mammal in need thereof comprising administering an inhibitor of p38 kinase in a therapeutically effective amount to prevent a facilitative state for sensory of pain in said mammal. In one embodiment the inhibitor is an inhibitor of p38α kinase. In one embodiment, the inhibitor exhibits an IC₅₀ value for p38α kinase that is at least ten fold less than the IC₅₀ value said inhibitor exhibits relative to other isoforms of p38 MAP kinase.

A method for preventing a facilitative state for sensation of pain in a mammal comprising administering an inhibitor of p38 kinase in a therapeutically effective amount to said mammal. In a preferred embodiment, the facilitative state comprises hyperalgesia. In yet another preferred embodiment, the facilitative state comprises allodynia.

A method for preventing a facilitative state for sensation of pain in a mammal comprising administering an inhibitor of p38 kinase in a therapeutically effective amount to said mammal.

Alternatively, the present invention provides a method to prevent or treat pain in a mammal in need thereof comprising administering an inhibitor of p38 kinase peripherally or systemically in a therapeutically effective amount to said mammal.

In another aspect, the present invention provides a method to prevent pain in a mammal in need thereof comprising administering an inhibitor of p38 kinase in a therapeutically effective amount to said mammal prior to a nociceptive event.

In yet another aspect, the present invention provides a method to prevent or treat pain in a mammal in need thereof comprising administering an inhibitor of p38 kinase in combination with an agent that inhibits pain and/or reduces inflammation in therapeutically effective amounts to said mammal.

The present invention also provides for a method of identifying a compound for preventing or treating pain in a mammal in need thereof, which comprises assaying candidate compounds for inhibition of p38 kinase activity, and identifying a compound that inhibits p38 kinase in a mammalian cell as indicative of a compound that alleviates or inhibits pain.

In another aspect, the present invention provides for a method to prevent or treat pain in a mammal in need thereof comprising administering a compound identified by the method of identifying a compound for alleviating or inhibiting pain in a mammal in need thereof, which comprises assaying candidate compounds for inhibition of p38 kinase activity, and identifying a compound that inhibits p38 kinase in a mammalian cell as indicative of a compound that alleviates or inhibits pain to the mammal.

### BRIEF DESCRIPTION OF THE SEVERAL VIEWS OF THE DRAWINGS

FIG. 1A. Thermal escape latency is plotted versus time after induction of thermal hyperalgesia by intrathecal (IT) injection of sP (30 nmol/10 μL) in rats pretreated (−10 min) with intrathecal saline, SD (60 kg) or SB203580 (SB) (30 μg). B. Percent of hyperalgesic index observed after IT SD or SB. (# indicates P<0.001 versus control (IT vehicle but no IT sP) and * indicates P<0.001 versus group receiving IT vehicle+IT sP). C. Western blots displaying bands for phosphorylated p38 MAPK (P-p38 MAPK) (Top) and total p38 MAPK (bottom) 10 minutes after intrathecal injection of saline or sP (30 nmol/10 μL). D. Representative Western blots showing COX-2 and COX-1 protein expression in spinal cord harvested 4 hours after IT injection of saline or sP (30 nmol/10 μL). Pretreatment but not posttreatment with IT SD (60 μg) (−10 min versus +5 min) prevented the increase in COX-2 expression. The lanes represents homogenates from different rats and (+) represents purified bovine COX-2 enzyme.

FIG. 2A. Flinching behavior plotted versus time following injection of formalin into the dorsal side of the left hindpaw of rats pretreated (−10 min) with intrathecal saline, SD (60 μg) or SB (30 μg). B. Cumulative number of flinches during Phase 2 (10-60 min, total number) observed after different doses of IT SD or SB. (p) indicates post-treatment, where SD was administrated intrathecally 5 minutes after the injection of paw formalin. C-F. Histochemical demonstration of FOS positive neurons in the ipsilateral dorsal horns at 2 hours following the intraplantar (IPLT) injection of formalin in the left paw of: (C) vehicle treated rat, (D) rat receiving IT vehicle and IPLT formalin or (E) rat receiving IT SD (60 μg) and IPLT formalin. (F) Spinal cord section from formalin treated rat but with no primary FOS antibody present under tissue processing. G. Histograms displaying the number of FOS-positive neurons in the ipsi- and contralateral dorsal horn of rats receiving IT Vehicle alone; IT vehicle+IPLT formalin or IT SD (60 μg)+IPLT formalin (n=4-6 rats per group, 10 sections per animal analyzed). Paw formalin resulted in a significant ipsilateral increase in FOS positive neurons #(p<0.001) and this increase was prevented by pre-treatment with IT SD *(p<0.001) vs. formalin alone).

FIG. 3. Thermal escape latency plotted versus time after the injection of IPLT carrageenan in rats pretreated (−10 min) with intrathecal vehicle, SD (60 μg) or SB (30 μg/10 μL). The control group received IT vehicle but no carrageenan. B. Hyperalgesic index observed after different doses of IT SD or SB. (#) represents P<0.001 versus control (IT vehicle but no carrageenan), (+) P<0.05 and (*) P<0.001 versus vehicle treated carrageenan injected group). C. Tactile thresholds (grams) measured in the ipsilateral paw after a thermal injury applied to the heel of one paw of the rats pretreated (−10 min) with intrathecal saline or SD (60 μg). D. Hyperalgesic index observed after different doses of IT SD. (p) indicates post treatment with IT SD (60 μg), SD was administrated 5 minutes after the thermal injury. (≠) represents P<0.001 versus control (IT vehicle but no thermal injury and (*) P<0.001 versus vehicle treated thermally injured group.

FIG. 4. P-p38 MAPK immunoreactivity (green fluorescence) in dorsal horn of lumbar spinal cord 10 minutes after (A) IT saline and (B) IT substance P (30 nmol/10 μL). A pronounced increase of p38 MAPK immunoractivity was seen in the superficial layers of the dorsal horn after IT sP. Spinal cord section incubated without primary antibody showing no unspecific binding of (C) anti-rabbit secondary antibody or (D) anti-mouse secondary antibody. (E) Colocalization of p38 MAPK and microglia-like structures. Sections were double labeled with anti-P-p38 MAPK (green) and a microglia marker anti-OX-43 (red). Close up of cell in dashed box showing (F) anti-OX-43 staining (red) and (G) anti-P-p38 (green) staining separately and (H) colocalized of OX-43 and P-p38 labeling (yellow). No colocalization was detected between p38 MAPK immunoreactivity (green) and (I) neuronal marker (NeuN), (J) oligodendrocytes marker (APC) or (K) astrocyte marker (GFAP) in dorsal horn of lumbar spinal cord 10 minutes after IT substance P (30 nmol/10 μL). The inserts in I and J are close-ups of cells present in the image. Section E-K was also stained with DAPI (blue), a nuclear marker.

FIG. 5. The escape latency plotted versus time after induction of hyperalgesia by intrathecal (IT) administration of NMDA (0.3 μg) in rats pretreated with 10 μg of SA versus control.

FIG. 6. Flinching behavior plotted versus time following induction of thermal hyperalgesis by intraplantar (IPLT) injection of carageenan into rat's hindpaw. SE was intravenously administered pre injury at indicated dosages.

FIG. 7. Flinching behavior plotted versus time following induction of thermal hyperalgesis by intraplantar (IPLT) injection of carageenan into rat's hindpaw. SD was administered intrathecally both prior to and after nociceptive event.

FIG. 8. Tabular data regarding effects of administration of SC.

FIG. 9. Graphical representation of paw withdrawal data from rats administered SC in a Randall Selitto Test.

FIG. 10. Graphical representation of paw withdrawal data from rats administered SC in a Plantar Test.

FIG. 11. Tabular data regarding effects of administration of SA.

FIG. 12A-B. Graphical representation of paw withdrawal data from rats administered SA in a Randall Selitto Test (A) and Plantar Test (B).

### DETAILED DESCRIPTION OF THE INVENTION

For clarity of disclosure, and not by way of limitation, the detailed description of the invention is divided into subsections.

### DEFINITIONS

Unless defined otherwise, all technical and scientific terms used herein have the same meaning as is commonly understood by one of ordinary skill in the art to which this invention belongs. All patents, patent applications, and other publications and sequences from GenBank and other databases referred to herein are incorporated by reference in their entirety. If a definition set forth in this section is contrary to or otherwise inconsistent with a definition found in such incorporated references, the definition set forth in this section prevails over the definition that is incorporated herein by reference.

As used herein, “a” or “an” means “at least one” or “one or more.”

As used herein, “allodynia” refers to a painful response to innocuous (non-painful) stimuli.

As used herein, “hyperalgesia” refers to an exaggerated response and/or sensitivity to painful stimuli.

As used herein, “IC₅₀” refers to an amount, concentration, or dosage of a particular test compound that achieves 50% inhibition of a maximal response in an assay that measure such a response.

As used herein, “nociceptive event” refers to painful or injurious stimuli directly or indirectly causing the transmission of pain.

As used herein, “preemptive analgesia” refers to the administration of anti-pain therapy prior to the first nociceptive event and, without being bound by any theory, likely preventing or reducing the activation of the nociceptors.

As used herein, “prevention or treatment of pain” refers to inhibition and/or alleviation of pain sensation.

As used herein, a “surgery” refers to the performance of an operation including, but not limited to, dental, reconstructive, cosmetic, and restorative procedures, as well as the removal of an organ or tissue or some portion thereof.

As used herein, a “therapeutically effective amount” refers to a concentration or amount that is effective upon administration to prevent or treat pain in a mammal.

Methods to Prevent or Treat Pain

The present invention provides a method to prevent or treat pain in a mammal by administering an inhibitor of p38 kinase in a therapeutically effective amount to said mammal.

Any mammal can be treated with the present method, including both human and animal subjects. Most preferably, humans are treated to prevent pain by administering the p38 inhibitor prior to a nociceptive event.

Any form of pain, chronic or acute, can be treated by the methods of the present invention. Pain states susceptible to treatment by the present method include, but are not limited to, neurological pain, neuropathies, polyneuropathies, diabetes-related polyneuropathies, headache (migraine and tension), trauma, neuralgias, post-zosterian neuralgia, trigeminal neuralgia, algodystrophy, HIV-related pain, musculo-skeletal pain, osteo-traumatic pain (e.g., bone fractures), arthritis, fibromyalgia, osteoarthritis, rheumatoid arthritis, spondylarthritis, phantom limb pain, back pain, vertebral pain, slipped disc surgery failure, post-surgery pain, cancer-related pain, vascular pain, Raynaud's syndrome, Horton's disease, arthritis, varicose ulcers, visceral pain, and childbirth.

Additionally, any form of anticipated pain may be prevented by the methods of the present invention. Preferably, the present method is used to prevent pain associated with surgery. Early intervention therapy is commonly known as preemptive analgesia, which reduces the hypersensitization of nociceptors by blocking pain impulses from ever reaching the brain.

Preemptive analgesia has received widespread acceptance as an adjunct to reduce perioperative pain in patients who undergo dental and surgical procedures, such as generally disclosed by Mayer et al. in U.S. Pat. No. 5,502,058. The technique is well accepted and is believed to involve the pharmacological interruption of afferent neurons to the dorsal horns of the spinal cord prior to the delivery of painful stimuli, such as a surgical incision. The anesthetic concept can be applied to most dental or surgical procedures, minimizing postoperative pain and the necessity for narcotic or parenteral analgesia, as well as reducing hospitalizations and required convalescence.

The pharmaceutical compositions utilized by the present invention comprise an inhibitor of p38 MAP kinase as an active ingredient, a pharmaceutically acceptable carrier and optionally other therapeutic ingredients or adjuvants. The pharmaceutical compositions may be conveniently presented in unit dosage form and prepared by any of the methods well known in the art of pharmacy.

Any known route of administration may used in the present invention. The compositions or compounds useful in the present invention may be administered orally, parenterally, topically, rectally, nasally, vaginally, or via implanted reservoir. Parenteral or systemic administration includes, but is not limited to, subcutaneous, intravenous, intraperitoneally, intramuscular, intra-articular, intra-synovial, intrasternol, intrathecal, intralesional, and intracranial injections. Preferably, the compositions or compounds of the present invention are administered orally, intrathecally or intraperitoneally/systemically.

Intrathecal administration allows the local administration of a compound to those regions of the spinal cord, such as to the dorsal horn regions, where polysynaptic relay of pain sensation occurs. Intrathecal administration, either via a bolus dosage or a constant infusion, delivers the compound directly to the subarachnoid space containing the cerebral spinal fluid (CSF).

Central delivery to spinal cord regions also can be effected by epidural injection to a region of the spinal cord exterior to the arachnoid membrane. It may be advantageous to add a means for enhancing permeation of the active compound through meningeal membranes. Such means are known in the art and include, but are not limited to, liposomal encapsulation, and the addition of a surfactant or an ion-pairing agent. Alternatively or additionally, increased arachnoid membrane permeation can be effected by administering a hypertonic dosing solution that increases permeability of meningeal barriers.

Administration by slow infusion is particularly useful when central routes such as intrathecal or epidural methods are employed. A number of implantable or body-mountable pumps useful in delivering compound at a regulated rate are known in the art. See, e.g., U.S. Pat. No. 4,619,652.

Any suitable formulation may be used. A compendium of art-known formulations is found in Remington's Pharmaceutical Sciences, latest edition, Mack Publishing Company, Easton, Pa.

The manner of administration and the formulation and dosage of the compounds useful in the invention depends on the nature of the condition, the severity of the condition, the particular subject to be treated, and the judgment of the practitioner; formulation will depend on mode of administration. Compounds useful in the present method can be administered pre-nociceptive event, post-nociceptive event, or some combination thereof. Compounds useful in the present invention can be administered once or more than once to a single patient in need of such treatment. The dosage of compound administered intrathecally can be 0.1 mg to 1 g/kg, preferably 1-100 mg/kg. The dosage of compound administered via the epideral route can be 0.1 μg to 1 mg/kg, preferably 1-100 μg/kg.

It should be noted that the attending physician would know how to and when to terminate, interrupt or adjust therapy to lower dosage due to adverse effects. Conversely, the physician also would know how to and when to adjust treatment to higher levels if the clinical response is not adequate.

Inhibitors of p38 MAP Kinase

As used herein, the term “inhibitor” includes any suitable molecule, compound, formulation or substance that may regulate p38 MAP kinase activity. The inhibitor may be a protein or fragment thereof, a small molecule compound, or even a nucleic acid molecule. It may affect a single p38 MAP kinase isoform or more than one isoform of p38 MAP kinase. In a preferred embodiment of the invention, the inhibitor regulates the α isoform of p38 MAP kinase.

According to the present invention, the inhibitor may exhibit its regulatory effect upstream or downstream of p38 MAP kinase or on p38 MAP kinase directly. Examples of inhibitor regulated p38 activity include those where the inhibitor may decrease transcription and/or translation of p38 MAP kinase, may decrease or inhibit post-translational modification and/or cellular trafficking of p38 MAP kinase, or may shorten the half-life of p38 MAP kinase. The inhibitor may also reversibly or irreversibly bind p38 MAP kinase, inhibit its activation, inactivate its enzymatic activity, or otherwise interfere with its interaction with downstream substrates.

If acting on p38 MAP kinase directly, the inhibitor should exhibit an IC₅₀ value of about 5 μM or less, preferably 500 nm or less, more preferably 100 nm or less. In a related embodiment, the inhibitor should exhibit an IC₅₀ value relative to the p38α isoform that is preferably at least ten fold less than that observed when the same inhibitor is tested against other p38 MAPK isoforms in the same or comparable assay.

To determine whether a candidate is an inhibitor useful for the treatment or prevention of pain in a mammal, an evaluation can be done on its p38 MAP kinase activity as well as its relative IC₅₀ value. This evaluation can be accomplished through a variety of convential in vitro assays. Such assays include those that assess inhibition of kinase or ATPase activity of activated p38 MAP kinase. The assays may also assess the ability of the inhibitor to bind p38 MAP kinase or to reduce or block an identified downstream effect of activated p38 MAP kinase, e.g., cytokine secretion.

For example, conventional binding assays are fairly inexpensive and simple to run. As previously mentioned, binding of a molecule to p38 MAP kinase, in and of itself, may be inhibitory, due to steric, allosteric or charge-charge interactions. A binding assay can be performed in solution or on a solid phase using p38 MAP kinase or a fragment thereof as a target. By using this as an initial screen, one can evaluate libraries of compounds for potential p38 regulatory activity.

The target may be either free in solution, fixed to a support, expressed in or on the surface of a cell. A label (ie. radioactive, fluorescent, quenching, et cetera.) can be placed on the target, compound, or both to determine presence or absence of binding. This approach can also be used to conduct a competitive binding assay to assess the inhibition of binding of a target to a natural or artificial substrate or binding partner. In any case, one may measure, either directly or indirectly, the amount of free label versus bound label to determine binding. There are many known variations and adaptations of this approach to minimize interference with binding activity and optimize signal.

For purposes of in vitro cellular assays, the compounds that represent potential inhibitors of p38 MAP kinase function can be administered to a cell in any number of ways. Preferably, the compound or composition can be added to the medium in which the cell is growing, such as tissue culture medium for cells grown in culture. The compound is provided in standard serial dilutions or in an amount determined by analogy to known modulators. Alternatively, the potential inhibitor may be encoded by a nucleic acid that is introduced into the cell wherein the cell essentially produces the potential inhibitor itself.

Alternative assays involving in vitro analysis of potential inhibitors include those where cells (HeLa) transfected with DNA coding for relevant kinases can be activated with substances such as sorbitol, IL-1, TNF, or PMA (phorbol myristate acetate). After immunoprecipitation of cell lysates, equal aliquots of immune complexes of the kinases are pre-incubated for an adequate time with a specific concentration of the potential inhibitor followed by addition of kinase substrate buffer mix containing labeled ATP and GST-ATF2 or MBP. After incubation, kinase reactions are ceased by the addition of SDS loading buffer. Phosphorylated substrate is resolved through SDS-PAGE and visualized and quantitated in a phosphorimager. Both p38 regulation, in terms of phosphorylation, and IC₅₀ values can be determined by quantitation. See, for example Kumar, S., McDonnell, P., Gum, R., Hand, A., Lee, J., and Young, P. (1997) Biochem. Biophys. Res. Commun. 235, 533-538.

Other in vitro assays may also assess the production of TNF-α as a correlate to p38 MAP kinase activity. One such example is a human whole blood assay. In this assay, venous blood is collected from healthy male volunteers into a heparinized syringe and is used within 2 hours of collection. Test compounds are dissolved in 100% DMSO and 1 μl aliquots of drug concentrations ranging from 0 to 1 mM are dispensed into quadruplicate wells of a 24-well microtiter plate (Nunclon Delta SI, Applied Scientific, So. San Francisco, Calif.). Whole blood is added at a volume of 1 ml/well and the mixture is incubated for 15 minutes with constant shaking (Titer Plate Shaker, Lab-Line Instruments, Inc., Melrose Park, Ill.) at a humidified atmosphere of 5% CO₂ at 37° C. Whole blood is cultured either undiluted or at a final dilution of 1:10 with RPMI 1640 (Gibco 31800+NaHCO₃, Life Technologies, Rockville, Md. and Scios, Inc., Sunnyvale, Calif.). At the end of the incubation period, 10 μl of LPS (E. coli 0111:B4, Sigma Chemical Co., St. Louis, Mo.) is added to each well to a final concentration of 1 or 0.1 μg/ml for undiluted or 1:10 diluted whole blood, respectively. The incubation is continued for an additional 2 hours. The reaction is stopped by placing the microtiter plates in an ice bath and plasma or cell-free supernates are collected by centrifugation at 3000 rpm for 10 minutes at 4° C. The plasma samples are stored at −80° C. until assayed for TNF-α levels by ELISA, following the directions supplied by Quantikine Human TNF-α assay kit (R&D Systems, Minneapolis, Minn.). IC₅₀ values are calculated using the concentration of inhibitor that causes a 50% decrease as compared to a control.

A similar assay is an enriched mononuclear cell assay. The enriched mononuclear cell assay, begins with cryopreserved Human Peripheral Blood Mononuclear Cells (HPBMCs) (Clonetics Corp.) that are rinsed and resuspended in a warm mixture of cell growth media. The resuspended cells are then counted and seeded at 1×10⁶ cells/well in a 24-well microtitre plate. The plates are then placed in an incubator for an hour to allow the cells to settle in each well. After the cells have settled, the media is aspirated and new media containing 100 ng/ml of the cytokine stimulatory factor lipopolysaccharide (LPS) and a test chemical compound is added to each well of the microtiter plate. Thus, each well contains HPBMCs, LPS and a test chemical compound. The cells are then incubated for 2 hours, and the amount of the cytokine Tumor Necrosis Factor Alpha (TNF-α) is measured using an enzyme linked immunosorbent assay (ELISA). One such ELISA for detecting the levels of TNF-α is commercially available from R&D Systems. The amount of TNF-α production by the HPBMCs in each well is then compared to a control well to determine whether the chemical compound acts as an inhibitor of cytokine production.

IC₅₀ values are calculated using the concentration of inhibitor that causes a 50% decrease as compared to a control.

Exemplary Inhibitors

Compounds useful in the practice of the present invention include, but are not limited to, compounds of formula: 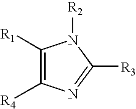
wherein

R₁ is a heteroaryl ring selected from 4-pyridyl, pyrimidinyl, quinolyl, isoquinolinyl, quinazolin-4-yl, 1-imidazolyl, 1-benzimidazolyl, 4-pyridazinyl, and a 1,2,4-triazin-5-yl ring, which heteroaryl ring is substituted one to three times with Y, N(R₁₀)C(O)R_{b}, a halo-substituted mono- or di-C₁₋₆ alkyl-substituted amino, or NHRₐ and which ring is further optionally substituted with C₁₋₄ alkyl, halogen, hydroxyl, optionally-substituted C₁₋₄ alkoxy, optionally-substituted C₁₋₄ alkylthio, optionally-substituted C₁₋₄ alkylsulfinyl, CH₂OR₁₂, amino, mono- and di-C₁₋₆ alkyl-substituted amino, NHRₐ, N(R₁₀)C(O)R_{b}, N(R₁₀)S(O)₂R_{d}, or an N-heterocyclyl ring which has from 5 to 7 members and optionally contains an additional heteroatom selected from oxygen, sulfur or NR₁₅;

Y is X₁—Rₐ;

X₁ is oxygen or sulfur;

Rₐ is C₁₋₆ alkyl, aryl, arylC₁₋₆ alkyl, heterocyclic, heterocyclylC₁₋₆ alkyl, heteroaryl, or heteroarylC₁₋₆ alkyl, wherein each of these moieties can be optionally substituted;

R_{b} is hydrogen, C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl;

R_{d} is C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl;

R₃ is hydrogen;

R₄ is phenyl, naphth-1-yl, naphth-2-yl, or a heteroaryl, which is optionally substituted by one or two substituents, each of which is independently selected, and which, for a 4-phenyl, 4-naphth-1-yl, 5-naphth-2-yl or 6-naphth-2-yl substituent, is halogen, cyano, nitro, —C(Z)NR₇R₁₇, —C(Z)OR₁₆, —(CR₁₀R₂₀)ᵥCOR₁₂, —SR₅, —SOR₅, —OR₁₂, halo-substituted-C₁₋₄ alkyl, C₁₋₄ alkyl, -ZC(Z)R₁₂, —NR₁₀C(Z)R₁₆, or —(CR₁₀R₂₀)ᵥNR₁₀R₂₀ and which, for other positions of substitution, is halogen, cyano, —C(Z)NR₁₃R₁₄, —C(Z)OR_{f}, —(CR₁₀R₂₀)_{m″}COR_{f}, —S(O)ₘR_{f}, —OR_{f}, —OR₁₂, halo-substituted C₁₋₄ alkyl, C₁₋₄ alkyl, —(CR₁₀R₂₀)_{m″}NR₁₀C(Z)R_{f}, —NR₁₀S(O)_{m′}R₈, —NR₁₀S(O)_{m′}NR₇R₁₇, -ZC(Z)R_{f}, -ZC(Z)R₁₂, or —(CR₁₀R₂₀)_{m″}NR₁₃R₁₄;

R_{f} is heterocyclyl, heterocyclylC₁₋₁₀ alkyl or R₈;

Z is oxygen or sulfur;

v is 0, 1, or 2;

m is 0, 1, or 2;

m′ is 1 or 2;

m″ is 0, 1, 2, 3, 4, or 5;

R₂ is C₁₋₁₀ alkyl N₃, —(CR₁₀R₂₀)_{n′}OR₉, heterocylyl, heterocycylC₁₋₁₀ alkyl, C₁₋₁₀ alkyl, halo-substituted C₁₋₁₀ alkyl, C₂₋₁₀ alkenyl, C₂₋₁₀ alkynyl, C₃₋₇ cycloalkyl, C₃₋₇ cycloalkylC₁₋₁₀ alkyl, C₅₋₇ cycloalkenyl, C₅₋₇cycloalkenylC₁₋₁₀ alkyl, aryl, arylC₁₋₁₀ alkyl, heteroaryl, heteroarylC₁₋₁₀ alkyl, (CR₁₀R₂₀)ₙOR₁₁, (CR₁₀R₂₀)ₙS(O)ₘR₁₈, (CR₁₀R₂₀)ₙNHS(O)₂R₁₈, (CR₁₀R₂₀)ₙNR₁₃R₁₄, (CR₁₀R₂₀)ₙNO₂, (CR₁₀R₂₀)ₙCN, (CR₁₀R₂₀)_{n′}SO₂R₁₈, (CR₁₀R₂₀)ₙS(O)_{m′}NR₁₃R₁₄, (CR₁₀R₂₀)ₙC(Z)R₁₁, (CR₁₀R₂₀)ₙOC(Z)R₁₁, (CR₁₀R₂₀)ₙC(Z)OR₁₁, (CR₁₀R₂₀)ₙC(Z)NR₁₃R₁₄, (CR₁₀R₂₀)ₙC(Z)NR₁₁OR₉, (CR₁₀R₂₀)ₙNR₁₀C(Z)R₁₁, (CR₁₀R₂₀)ₙNR₁₀C(Z)NR₁₃R₁₄, (CR₁₀R₂₀)ₙN(OR₆)C(Z)NR₁₃R₁₄, (CR₁₀R₂₀)ₙN(OR₆)C(Z)R₁₁, (CR₁₀R₂₀)ₙC(═NOR₆)R₁₁, (CR₁₀R₂₀)ₙNR₁₀C(═NR₁₉)NR₁₃R₁₄, (CR₁₀R₂₀)ₙOC(Z)NR₁₃R₁₄, (CR₁₀R₂₀)ₙNR₁₀C(Z)NR₁₃R₁₄, (CR₁₀R₂₀)ₙNR₁₀C(Z)OR₁₀, 5-(R₁₈)-1,2,4-oxadiazol-3-yl or 4-(R₁₂)-5-(R₁₈R₁₉)-4,5-dihydro-1,2,4-oxadiazol-3-yl; wherein the aryl, arylalkyl, heteroaryl, heteroaryl alkyl, cycloalkyl, cycloalkyl alkyl, heterocyclic and heterocyclic alkyl groups can be optionally substituted;

n is an integer having a value of 1 to 10;

n′ is 0, or an integer having a value of 1 to 10;

R₅ is hydrogen, C₁₋₄ alkyl, C₂₋₄ alkenyl, C₂₋₄ alkynyl or NR₇R₁₇, excluding the moieties —SR₅ being —SNR₇R₁₇ and —S(O)R₅ being —SOH;

R₆ is hydrogen, a pharmaceutically-acceptable cation, C₁₋₁₀ alkyl, C₃₋₇ cycloalkyl, aryl, arylC₁₋₄ alkyl, heteroaryl, heteroarylC₁₋₁₀ alkyl, heterocyclyl, aroyl, or C₁₋₁₀ alkanoyl;

R₇ and R₁₇ are each independently selected from hydrogen or C₁₋₄ alkyl, or R₇ and R₁₇ together with the nitrogen to which they are attached form a heterocyclic ring of 5 to 7 members which ring optionally contains an additional heteroatom selected from oxygen, sulfur or NR₁₅;

R₈ is C₁₋₁₀ alkyl, halo-substituted C₁₋₁₀ alkyl, C₂₋₁₀ alkenyl, C₂₋₁₀ alkynyl, C₃₋₇ cycloalkyl, C₅₋₇ cycloalkenyl, aryl, arylC₁₋₁₀ alkyl, heteroaryl, heteroarylC₁₋₁₀ alkyl, (CR₁₀R₂₀)ₙOR₁₁, (CR₁₀R₂₀)ₙS(O)ₘR₁₈, (CR₁₀R₂₀)ₙNHS(O)₂R₁₈, or (CR₁₀R₂₀)ₙNR₁₃R₁₄, wherein the aryl, arylalkyl, heteroaryl, and heteroaryl alkyl can be optionally substituted;

R₉ is hydrogen, —C(Z)R₁₁, optionally-substituted C₁₋₁₀ alkyl, S(O)₂R₁₈, optionally-substituted aryl or optionally-substituted arylC₁₋₄ alkyl;

R₁₀ and R₂₀ are each independently selected from hydrogen or C₁₋₄ alkyl;

R₁₁ is hydrogen, C₁₋₁₀ alkyl, C₃₋₇ cycloalkyl, heterocyclyl, heterocyclylC₁₋₁₀ alkyl, aryl, arylC₁₋₁₀ alkyl, heteroaryl or heteroarylC₁₋₁₀ alkyl;

R₁₂ is hydrogen or R₁₆;

R₁₃ and R₁₄ are each independently selected from hydrogen or optionally-substituted C₁₋₄ alkyl, optionally-substituted aryl or optionally-substituted arylC₁₋₄ alkyl, or together with the nitrogen to which they are attached form a heterocyclic ring of 5 to 7 members which ring optionally contains an additional heteroatom selected from oxygen, sulfur or NR₉;

R₁₅ is R₁₀ or C(Z)C₁₋₄ alkyl;

R₁₆ is C₁₋₄ alkyl, halo-substituted C₁₋₄ alkyl, or C₃₋₇ cycloalkyl;

R₁₈ is C₁₋₁₀ alkyl, C₃₋₇ cycloalkyl, heterocyclyl, aryl, arylC₁₋₁₀ alkyl, heterocyclyl, heterocyclylC₁₋₁₀ alkyl, heteroaryl or heteroarylC₁₋₁₀ alkyl; and

R₁₉ is hydrogen, cyano, C₁₋₄ alkyl, C₃₋₇ cycloalkyl or aryl;

or a pharmaceutically-acceptable salt thereof,

or wherein

R₁, Y, X₁, Rₐ, R_{b}, R_{d}, v, m, m′, m″, Z, n, n′, and R₅ are defined as above, and

R₂ is hydrogen, C₁₋₁₀ alkyl, halo-substituted C₁₋₁₀ alkyl, C₂₋₁₀ alkenyl, C₂₋₁₀ alkynyl, C₃₋₇ cycloalkyl, C₃₋₇ cycloalkylC₁₋₁₀ alkyl, C₅₋₇ cycloalkenyl, aryl, arylC₁₋₁₀ alkyl, heteroaryl, heteroarylC₁₋₁₀ alkyl, heterocyclyl, heterocyclylC₁₋₁₀ alkyl, (CR₁₀R₂₈)ₙOR₁₂, (CR₁₀R₂₈)_{n′}OR₁₃, (CR₁₀R₂₈)_{n′}S(O)ₘR₂₅, (CR₁₀R₂₈)ₙS(O)₂R₂₅, (CR₁₀R₂₈)_{n′}NHS(O)₂R₂₅, (CR₁₀R₂₈)_{n′}NR₈R₉, (CR₁₀R₂₈)_{n′}NO₂, (CR₁₀R₂₈)_{n′}CN, (CR₁₀R₂₈)_{n′}S(O)ₘNR₈R₉, (CR₁₀R₂₈)_{n′}C(Z)R₁₃, (CR₁₀R₂₈)_{n′}C(Z)OR₁₃, (CR₁₀R₂₈)_{n′}C(Z)NR₈R₉, (CR₁₀R₂₈)_{n′}C(Z)NR₁₃OR₁₂, (CR₁₀R₂₈)_{n′}NR₁₀C(Z)R₁₃, (CR₁₀R₂₈)_{n′}NR₁₀C(Z)NR₈R₉, (CR₁₀R₂₈)_{n′}N(OR₂₁)C(Z)NR₈R₉, (CR₁₀R₂₈)_{n′}N(OR₂₁)C(Z)R₁₃, (CR₁₀R₂₈)_{n′}C(═NOR₂₁)R₁₃, (CR₁₀R₂₈)_{n′}NR₁₀C(═NR₂₇)NR₈R₉, (CR₁₀R₂₈)_{n′}OC(Z)NR₈R₉, (CR₁₀R₂₈)_{n′}NR₁₀C(Z)OR₁₀, (CR₁₀R₂₈)_{n′}NR₁₀C(Z)OR₁₀, 5-(R₂₅)-1,2,4-oxadiazol-3-yl or 4-(R₁₂)-5-(R₁₈R₁₉)-4,5-dihydro-1,2,4-oxadiazol-3-yl; wherein the cycloalkyl, cycloalkyl alkyl, aryl, arylalkyl, heteroaryl, heteroarylalkyl, heterocyclyl, or heterocyclylalkyl moieties can be optionally substituted;

R₃ is hydrogen or Q-(Y₁)ₜ;

Q is an aryl or heteroaryl group;

t is 1, 2, or 3;

Y₁ is independently selected from hydrogen, C₁₋₅ alkyl, halo-substituted C₁₋₅ alkyl, halogen, or —(CR₁₀R₂₀)ₙY₂;

Y₂ is OR₈, NO₂, S(O)_{m″}R₁₁, SR₈, S(O)_{m″}OR₈, S(O)ₘNR₈R₉, NR₈R₉, O(CR₁₀R₂₀), NR₈R₉, C(O)R₈, CO₂R₈, CO₂(CR₁₀R₂₀)_{n′}CONR₈R₉, ZC(O)R₈, CN, C(Z)NR₈R₉, NR₁₀C(Z)R₈, C(Z)NR₈OR₉, NR₁₀C(Z)NR₈R₉, NR₁₀S(O)_{m″}R₁₁, N(OR₂₁)C(Z)NR₈R₉, N(OR₂₁)C(Z)R₈, C(═NOR₂₁)R₈, NR₁₀C(═NR₁₅)SR₁₁, NR₁₀C(═NR₅)NR₈R₉, NR₁₀C(═CR₁₄R₂₄)SR₁₁, NR₁₀C(═CR₁₄R₂₄)NR₈R₉, NR₁₀C(O)C(O)NR₈R₉, NR₁₀C(O)C(O)OR₁₀, C(═NR₁₃)NR₈R₉, C(═NOR₁₃)NR₈R₉, C(═NR₁₃)ZR₁₁, OC(Z)NR₈R₉, NR₁₀S(O)_{m″}CF₃, NR₁₀C(Z)OR₁₀, 5-(R₁₈)-1,2,4-oxadiazol-3-yl or 4-(R₁₂)-5-(R₁₈R₁₉)-4,5-dihydro-1,2,4-oxadiazol-3-yl;

R₄ is phenyl, naphth-1-yl or naphth-2-yl which is optionally substituted by one or two substituents, each of which is independently selected, and which, for a 4-phenyl, 4-naphth-1-yl or 5-naphth-2-yl substituent, is halo, nitro, cyano, C(Z)NR₇R₁₇, C(Z)OR₂₃, (CR₁₀R₂₀)ᵥCOR₃₆, SR₅, SOR₅, OR₃₆, halo-substituted-C₁₋₄ alkyl, C₁₋₄ alkyl, ZC(Z)R₃₆, NR₁₀C(Z)R₂₃, or (CR₁₀R₂₀)ᵥNR₁₀R₂₀ and which, for other positions of substitution, is halo, nitro, cyano, C(Z)NR₁₆R₂₆, C(Z)OR₈, (CR₁₀R₂₀)_{m″}COR₈, S(O)ₘR₈, OR₈, halo-substituted-C₁₋₄ alkyl, C₁₋₄ alkyl, (CR₁₀R₂₀)_{m″}NR₁₀C(Z)R₈, NR₁₀S(O)_{m′}R₁₁, NR₁₀S(O)_{m′}NR₇R₁₇, ZC(Z)R₈ or (CR₁₀R₂₀)_{m″}NR₁₆R₂₆;

R₇ and R₁₇ are each independently selected from hydrogen or C₁₋₄ alkyl, or R₇ and R₁₇ together with the nitrogen to which they are attached form a heterocyclic ring of 5 to 7 members, which ring optionally contains an additional heteroatom selected from oxygen, sulfur or NR₂₂;

R₈ is hydrogen, heterocyclyl, heterocyclylalkyl or R₁₁;

R₉ is hydrogen, C₁₋₁₀ alkyl, C₂₋₁₀ alkenyl, C₂₋₁₀ alkynyl, C₃₋₇ cycloalkyl, C₅₋₇ cycloalkenyl, aryl, arylalkyl, heteroaryl or heteroarylalkyl, or R₈ and R₉ can together with the nitrogen to which they are attached form a heterocyclic ring of 5 to 7 members, which ring optionally contains an additional heteroatom selected from oxygen, sulfur or NR₁₂;

R₁₀ and R₂₀ are each independently selected from hydrogen or C₁₋₄ alkyl;

R₁₁ is C₁₋₁₀ alkyl, halo-substituted C₁₋₁₀ alkyl, C₂₋₁₀ alkenyl, C₂₋₁₀ alkynyl, C₃₋₇ cycloalkyl, C₅₋₇ cycloalkenyl, aryl, arylalkyl, heteroaryl or heteroarylalkyl;

R₁₂ is hydrogen, —C(Z)R₁₃ or optionally-substituted C₁₋₄ alkyl, optionally-substituted aryl, optionally-substituted arylC₁₋₄ alkyl, or S(O)₂R₂₅;

R₁₃ is hydrogen, C₁₋₁₀ alkyl, C₃₋₇ cycloalkyl, heterocyclyl, heterocyclylC₁₋₁₀ alkyl, aryl, arylC₁₋₁₀ alkyl, heteroaryl or heteroaryl C₁₋₁₀ alkyl, wherein all of these moieties can be optionally substituted;

R₁₄ and R₂₄ are each independently selected from hydrogen, alkyl, nitro or cyano;

R₁₅ is hydrogen, cyano, C₁₋₄ alkyl, C₃₋₇ cycloalkyl or aryl;

R₁₆ and R₂₆ are each independently selected from hydrogen or optionally-substituted CIA alkyl, optionally-substituted aryl or optionally-substituted arylC₁₋₄ alkyl, or together with the nitrogen to which they are attached form a heterocyclic ring of 5 to 7 members, which ring optionally contains an additional heteroatom selected from oxygen, sulfur or NR₁₂;

R₁₈ and R₁₉ are each independently selected from hydrogen, C₁₋₄ alkyl, substituted alkyl, optionally-substituted aryl, optionally-substituted arylalkyl, or together denote an oxygen or sulfur;

R₂₁ is hydrogen, a pharmaceutically-acceptable cation, C₁₋₁₀ alkyl, C₃₋₇ cycloalkyl, aryl, arylC₁₋₄ alkyl, heteroaryl, heteroarylalkyl, heterocyclyl, aroyl, or C₁₋₁₀ alkanoyl;

R₂₂ is R₁₀ or C(Z)-C₁₋₄ alkyl;

R₂₃ is C₁₋₄ alkyl, halo-substituted-C₁₋₄ alkyl, or C₃₋₅ cycloalkyl;

R₂₅ is C₁₋₁₀ alkyl, C₃₋₇ cycloalkyl, heterocyclyl, aryl, arylalkyl, heterocyclyl, heterocyclylC₁₋₁₀ alkyl, heteroaryl or heteroarylalkyl;

R₂₇ is hydrogen, cyano, C₁₋₄ alkyl, C₃₋₇ cycloalkyl, or aryl;

R₂₈ is hydrogen, C₁₋₆ alkyl, C₃₋₇ cycloalkyl, aryl, arylC₁₋₄ alkyl, heteroaryl, heteroarylC₁₋₄alkyl, heterocyclyl, or heterocyclylC₁₋₄ alkyl moiety, all of which can be optionally substituted; and

R₃₆ is hydrogen or R₂₃;

or a pharmaceutically acceptable salt thereof.

Exemplary compounds of this formula include:
1-[3-(4-morpholinyl)propyl]-4-(4-fluorophenyl)-5-(4-pyridyl)imidazole;
1-(3-chloropropyl)-4-(4-fluorophenyl)-5-(4-pyridyl)imidazole;
1-(3-azidopropyl)-4-(4-fluorophenyl)-5-(4-pyridyl)imidazole;
1-(3-aminopropyl)-4-(4-fluorophenyl)-5-(4-pyridyl)imidazole;
1-(3-methylsulfonamidopropyl)-4-(4-fluorophenyl)-5-(4-pyridyl)imidazole;
1-[3-(N-phenylmethyl)aminopropyl]-4-(4-fluorophenyl)-5-(4-pyridyl)imidazole;
1-[3-(N-phenylmethyl-N-methyl)aminopropyl]-4-(4-fluorophenyl)-5-(4-pyridyl)imidazole;
1-[3-(1-pyrrolidinyl)propyl]-4-(4-fluorophenyl)-5-(4-pyridyl)imidazole;
1-(3-diethylaminopropyl)-4-(4-fluorophenyl)-5-(4-pyridyl)imidazole;
1-[3-(1-piperidinyl)propyl]-4-(4-fluorophenyl)-5-(4-pyridyl)imidazole;
1-[3-(methylthio)propyl]-4-(4-fluorophenyl)-5-(4-pyridyl)imidazole;
1-[2-(4-morpholinyl)ethyl]-4-(4-fluorophenyl)-5-(4-pyridyl)imidazole;
1-[3-(4-morpholinyl)propyl]-4-(3-methylthiophenyl)-5-(4-pyridyl)imidazole;
(+/−)-1-[3-(4-morpholinyl)propyl]-4-(3-methylsulfinylphenyl)-5-(4-pyridyl)imidazole;
1-[3-(N-methyl-N-benzyl)aminopropyl]-4-(3-methylthiophenyl)-5-(4-pyridyl)imidazole;
1-[3-(N-methyl-N-benzyl)aminopropyl]-4-(3-methylsulfinylphenyl)-5-(4-pyridyl)imidazole;
1-[4-(methylthio)phenyl]-4-(4-fluorophenyl)-5-(4-pyridyl)imidazole;
1-[4-(methylsulfinyl)phenyl]-4-(4-fluorophenyl)-5-(4-pyridyl)imidazole;
1-[3-(methylthio)phenyl]-4-(4-fluorophenyl)-5-(4-pyridyl)imidazole;
(+/−)-1-[3-(methylsulfinyl)phenyl]-4-(4-fluorophenyl)-5-(4-pyridyl)imidazole;
1-[2-(methylthio)phenyl]-4-(4-fluorophenyl)-5-(4-pyridyl)imidazole;
1-[2-(methylsulfinyl)phenyl]-4-(4-fluorophenyl)-5-(4-pyridyl)imidazole;
1-[4-(4-morpholinyl)butyl]-4-(4-fluorophenyl)-5-(4-pyridyl)imidazole;
1-cyclopropyl-4-(4-fluorophenyl)-5-(4-pyridyl)imidazole;
1-isopropyl-4-(4-fluorophenyl)-5-(4-pyridyl)imidazole;
1-cyclopropylmethyl-4-(4-fluorophenyl)-5-(4-pyridyl)imidazole;
1-tert-butyl-4-(4-fluorophenyl)-5-(4-pyridyl)imidazole;
1-(2,2-diethoxyethyl)-4-(4-fluorophenyl)-5-(4-pyridyl)imidazole;
1-formylmethyl-4-(4-fluorophenyl)-5-(4-pyridyl)imidazole;
1-hydroxyiminylmethyl-4-(4-fluorophenyl)-5-(4-pyridyl)imidazole;
1-cyanomethyl-4-(4-fluorophenyl)-5-(4-pyridyl)imidazole;
1-[3-(4-morpholinyl)propyl)-4-(4-fluorophenyl)-5-(2-methylpyrid-4-yl)imidazole;
4-(4-fluorophenyl)-1-[3-(4-morpholinyl)propyl]-5-(2-chloropyridin-4-yl)imidazole;
4-(4-fluorophenyl)-1-[3-(4-morpholinyl)propyl]-5-(2-amino-4-pyridinyl)imidazole;
1-(4-carboxymethyl)propyl-4-(4-fluorophenyl)-5-(4-pyridyl)imidazole;
1-(4-carboxypropyl)-4-(4-fluorophenyl)-5-(4-pyridyl)imidazole;
1-(3-carboxymethyl)ethyl-4-(4-fluorophenyl)-5-(4-pyridyl)imidazole;
1-(3-carboxy)ethyl-4-(4-fluorophenyl)-5-(4-pyridyl)imidazole;
1-(1-benzylpiperidin-4-yl)-4-(4-fluorophenyl)-5-(4-pyridyl)imidazole;
5-(2-aminopyrimidin-4-yl)-4-(4-fluorophenyl)-1-[3-(4-morpholinyl)propyl]imidazole;
5-(2-aminopyrimidin-4-yl)-4-(4-fluorophenyl)-1-(1-benzylpiperidin-4-yl)imidazole;
5-(2-aminopyrimidin-4-yl)-4-(4-fluorophenyl)-1-(2-propyl)imidazole;
5-(2-aminopyrimidin-4-yl)-4-(4-fluorophenyl)-1-(cyclopropylmethyl)imidazole;
5-(2-aminopyrimidin-4-yl)-4-(4-fluorophenyl)-1-(1-carboxyethyl-4-piperidinyl)imidazole;
5-(2-aminopyrimidin-4-yl)-4-(4-fluorophenyl)-1-(4-piperidinyl)imidazole;
1-methyl-4-phenyl-5-(4-pyridyl)imidazole;
1-methyl-4-[3-(chlorophenyl)]-5-(4-pyridinyl)imidazole;
1-methyl-4-(3-methylthiophenyl)-5-(4-pyridyl)imidazole;
(+/−)-1-methyl-4-(3-methylsulfinylphenyl)-5-(4-pyridyl)imidazole;
(+/−)-4-(4-fluorophenyl)-1-[3-(methylsulfinyl)propyl]-5-(4-pyridinyl)imidazole;
4-(4-fluorophenyl)-1-[(3-methylsulfonyl)propyl]-5-(4-pyridinyl)imidazole;
1-(3-phenoxypropyl)-4-(4-fluorophenyl)-5-(4-pyridinyl)imidazole;
1-[3-(phenylthio)propyl]-4-(4-fluorophenyl)-5-(4-pyridinyl)imidazole;
1-[3-(4-morpholinyl)propyl]-4-(4-fluorophenyl)-5-(4-quinolyl)imidazole;
(+/−)-1-(3-phenylsulfinylpropyl)-4-(4-fluorophenyl)-5-(4-pyridinyl)imidazole;
1-(3-ethoxypropyl)-4-(4-fluorophenyl)-5-(4-pyridinyl)imidazole;
1-(3-phenylsulfonylpropyl-4-(4-fluorophenyl)-5-(4-pyridinyl)imidazole;
1-[3-(4-morpholinyl)propyl]-4-(3-chlorophenyl)-5-(4-pyridyl)imidazole;
1-[3-(4-morpholinyl)propyl]-4-(3,4-dichlorophenyl)-5-(4-pyridyl)imidazole;
4-[4-(4-fluorophenyl)-1-[3-(4-morpholinyl)propyl]-5-(pyrimid-2-one-4-yl)imidazole;
4-(4-fluorophenyl)-5-[2-(methylthio)-4-pyrimidinyl]-1-[3-(4-morpholinyl)propyl]imidazole;
(+/−)-4-(4-fluorophenyl)-5-[2-(methylsulfinyl)-4-pyrimidinyl]-1-[3-(4-morpholinyl)propyl]imidazole;
1-(1-propenyl)-4-(4-fluorophenyl)-5-(4-pyridinyl)imidazole;
1-(2-propenyl)-4-(4-fluorophenyl)-5-(4-pyridinyl)imidazole;
5-[(2-N,N-dimethylamino)pyrimidin-4-yl]-4-(4-fluorophenyl)-1-[3-(4-morpholinyl)propyl]imidazole;
1-[3-(4-morpholinyl)propyl]-5-(4-pyridinyl)-4-[4-(trifluoromethyl)phenyl]imidazole;
1-[3-(4-morpholinyl)propyl]-5-(4-pyridinyl)-4-[3-(trifluoromethyl)phenyl]imidazole;
1-(cyclopropylmethyl)-4-(3,4-dichlorophenyl)-5-(4-pyridinyl)imidazole;
1-(cyclopropylmethyl)-4-(3-trifluoromethylphenyl)-5-(4-pyridinyl)imidazole;
1-(cyclopropylmethyl)-4-(4-fluorophenyl)-5-(2-methylpyrid-4-yl)imidazole;
1-[3-(4-morpholinyl)propyl]-5-(4-pyridinyl)-4-(3,5-bistrifluoromethylphenyl)imidazole;
5-[4-(2-aminopyrimidinyl)]-4-(4-fluorophenyl)-1-(2-carboxy-2,2-dimethylethyl)imidazole;
1-(1-formyl-4-piperidinyl)-4-(4-fluorophenyl)-5-(4-pyridinyl)imidazole;
5-(2-amino-4-pyrimidinyl)-4-(4-fluorophenyl)-1-(1-methyl-4-piperidinyl)imidazole;
1-(2,2-dimethyl-3-morpholin-4-yl)propyl-4-(4-fluorophenyl)-5-(2-amino-4-pyrimidinyl)imidazole;
4-(4-fluorophenyl)-5-(4-pyridyl)-1-(2-acetoxyethyl)imidazole;
5-(2-aminopyrimidin-4-yl)-4-(4-fluorophenyl)-1-(1-benzylpyrrolin-3-yl)imidazole;
5-(2-aminopyrimidin-4-yl)-4-(4-fluorophenyl)-1-(2,2,6,6-tetramethylpiperidin-4-yl)imidazole;
5-[4-(2-N-methylamino)pyrimidinyl]-4-(4-fluorophenyl)-1-(4-N-methylpiperidine)imidazole;
5-[4-(2-N-methylamino)pyrimidinyl]-4-(4-fluorophenyl)-1-(4-N-morpholino-1-propyl)imidazole;
5-[4-(2-N-methylamino)pyrimidinyl)-4-(4-fluorophenyl)-1-(4-piperidine)imidazole;
5-[(2-ethylamino)pyrimidin-4-yl]-4-(4-fluorophenyl)-1-(1-methylpiperidin-4-yl)imidazole;
4-(4-fluorophenyl)-5-[2-(isopropyl)aminopyrimidin-4-yl]-1-(1-methylpiperidin-4-yl)imidazole;
5-(2-acetamido-4-pyrimidinyl)-4-(4-fluorophenyl)-1-(4-N-morpholino-1-propyl)imidazole;
5-(2-acetamido-4-pyrimidinyl)-4-(4-fluorophenyl)-1-(1-methyl-4-piperidinyl)imidazole;
5-[4-(2-N-methylthio)pyrimidinyl]-4-(4-fluorophenyl)-1-(4-piperidine)imidazole;
4-(fluorophenyl)-1-(methyl-4-piperidinyl)-5-(2-methylthio-4-pyrimidinyl)imidazole;
4-(fluorophenyl)-1-(methyl-4-piperidinyl)-5-(2-methysulfinyl-4-pyrimidinyl)imidazole;
1-tert-butyl-4-(4-fluorophenyl)-5-(2-methysulfinyl-4-pyrimidinyl)imidazole;
5-[4-(2-aminopyrimidinyl)]-4-(4-fluorophenyl)-1-(2,2,6,6-tetramethyl-4-piperidinyl)imidazole;
5-[4-(2-N-methylamino-4-pyrimidinyl)]-4-(4-fluorophenyl)-1-(2,2,6,6-tetramethyl-4-piperidine)imidazole;
5-(2-amino-4-pyrimidinyl)-4-(4-fluorophenyl)-1-(tetrahydro-4-thiopyranyl)imidazole;
5-(2-amino-4-pyrimidinyl)-4-(4-fluorophenyl)-1-(tetrahydro-4-pyranyl)imidazole;
5-(2-methylamino-4-pyrimidinyl)-4-(4-fluorophenyl)-1-(2-cyanoethyl)imidazole;
5-(2-amino-4-pyrimidinyl)-4-(4-fluorophenyl)-1-(tetrahydro-4-sulfinylpyranyl)imidazole;
5-(2-amino-4-pyrimidinyl)-4-(4-fluorophenyl)-1-(tetrahydro-4-sulfonylpyranyl)imidazole;
5-(2-methylamino-4-pyrimidinyl)-4-(4-fluorophenyl)-1-(2,2,2-trifluoroethyl-4-piperidinyl)imidazole;
5-(2-amino-4-pyrimidinyl)-4-(4-fluorophenyl)-1-(trifluoroacetyl-4-piperidinyl)imidazole;
5-(4-pyridyl)-4-(4-fluorophenyl)-1-(4-piperidinyl)imidazole;
5-(4-pyridyl)-4-(4-fluorophenyl)-1-(1-t-butoxycarbonyl-4-piperidinyl)imidazole;
5-(2-amino-4-pyrimidinyl)-4-(4-fluorophenyl)-1-(4-(1,3-dioxycyclopentyl)cyclohexyl)imidazole;
5-(2-amino-4-pyrimidinyl)-4-(4-fluorophenyl)-1-(4-ketocyclohexyl)imidazole;
5-(2-amino-4-pyrimidinyl)-4-(4-fluorophenyl)-1-(4-cyclohexyl oxime) imidazole;
5-(2-amino-4-pyrimidinyl)-4-(4-fluorophenyl)-1-(4-cyclohexyl hydroxylamine) imidazole;
5-(2-amino-4-pyrimidinyl)-4-(4-fluorophenyl)-1-(trans-4-hydroxyurea) imidazole;
5-(2-amino-4-pyrimidinyl)-4-(4-fluorophenyl)-1-(cis-4-hydroxyurea) imidazole;
5-(2-amino-4-pyrimidinyl)-4-(4-fluorophenyl)-1-(4-hydroxycyclohexyl)imidazole;
5-[4-(2-N-methylamino)pyrimidinyl]-4-(4-fluorophenyl)-1-(4-ketocyclohexyl)imidazole;
5-[4-(2-N-methylamino)pyrimidinyl]-4-(4-fluorophenyl)-1-(trans-4-hydroxycyclohexyl)imidazole;
5-[4-(2-N-methylamino)pyrimidinyl]-4-(4-fluorophenyl)-1-(cis-4-hydroxycyclohexyl)imidazole;
5-[4-(2-N-methylamino)pyrimidinyl]-4-(4-fluorophenyl)-1-[4-(cis-pyrrolidinyl)cyclohexyl]imidazole;
5-[4-(2-N-methylamino)pyrimidinyl]-4-(4-fluorophenyl)-1-[4-(trans-1-pyrrolidinyl)cyclohexyl]imidazole;
5-[4-(2-N-methylamino)pyrimidinyl]-4-(4-fluorophenyl)-1-(4-ethynyl-4-hydroxycyclohexyl)imidazole;
5-[4-(2-N-methylamino)pyrimidinyl]-4-(4-fluorophenyl)-1-(4-(1-propynyl)-4-hydroxycyclohexyl)imidazole;
5-[4-(2-N-methylamino)pyrimidinyl]-4-(4-fluorophenyl)-1-(4-amino-4-methylcyclohexyl)imidazole;
5-[4-(2-N-methylamino)pyrimidinyl]-4-(4-fluorophenyl)-1-(4-acetamido-4-methylcyclohexyl)imidazole;
5-[4-(2-N-methylamino)pyrimidinyl]-4-(4-fluorophenyl)-1-(4-hydroxy-4-methylcyclohexyl)imidazole;
5-[4-(2-N-methylamino)pyrimidinyl]-4-(4-fluorophenyl)-1-(4-oxiranylcyclohexyl)imidazole;
5-[4-(2-N-methylamino)pyrimidinyl]-4-(4-fluorophenyl)-1-(4-cyanomethyl-4-hydroxycyclohexyl)imidazole;
5-[4-(2-N-methylamino)pyrimidinyl]-4-(4-fluorophenyl)-1-(4-hydroxy-4-hydroxymethylcyclohexyl)imidazole;
5-[4-(2-amino)pyrimidinyl]-4-(4-fluorophenyl)-1-[4-hydroxy-4-(1-propynyl)cyclohexyl]imidazole;
5-[4-(2-amino)pyrimidinyl]-4-(4-fluorophenyl)-1-(4-hydroxy-4-methylcyclohexyl)imidazole;
5-[4-(2-N-methylamino)pyrimidinyl]-4-(4-fluorophenyl)-1-(4-hydroxy-4-isopropylcyclohexyl)imidazole;
5-[4-(2-N-methylamino)pyrimidinyl]-4-(4-fluorophenyl)-1-(4-hydroxy-4-phenylcyclohexyl)imidazole;
5-[4-(2-N-methylamino)pyrimidinyl]-4-(4-fluorophenyl)-1-(4-hydroxy-4-benzylcyclohexyl)imidazole;
5-[4-(2-N-methylamino)pyrimidinyl]-4-(4-fluorophenyl)-1-(4-hydroxy-4-cyanomethyl cyclohexyl)imidazole;
5-[4-(2-N-methylamino)pyrimidinyl]-4-(4-fluorophenyl)-1-(4-hydroxy-4-(2-cyanoethyl)cyclohexyl)imidazole;
5-[4-(2-N-methylamino)pyrimidinyl]-4-(4-fluorophenyl)-1-(4-hydroxy-4-(2-aminoethyl)cyclohexyl)imidazole;
5-[4-(2-N-methylamino)pyrimidinyl]-4-(4-fluorophenyl)-1-(4-hydroxy-4-(2-nitroethyl)cyclohexyl)imidazole;
5-[4-(2-N-methylamino)pyrimidinyl]-4-(4-fluorophenyl)-1-(4-hydroxymethyl-4-aminocyclohexyl)imidazole;
5-[4-(2-N-methylamino)pyrimidinyl]-4-(4-fluorophenyl)-1-(4-hydroxy-4-aminocyclohexyl)imidazole;
5-[4-(2-N-methylamino)pyrimidinyl]-4-(4-fluorophenyl)-1-(4-aminocyclohexyl)imidazole;
5-[4-(2-N-methylamino)pyrimidinyl]-4-(4-fluorophenyl)-1-(4-hydroxy-4-thiomethyl cyclohexyl)imidazole;
5-[4-(2-N-methylamino)pyrimidinyl]-4-(4-fluorophenyl)-1-(4-hydroxy-4-hydroxy methylcyclohexyl)imidazole;
5-[4-(2-N-methylamino)pyrimidinyl]-4-(4-fluorophenyl)-1-(4-hydroxy-4-aminomethylcyclohexyl)imidazole;
5-[4-(2-amino)pyrimidinyl]-4-(4-fluorophenyl)-1-(4-amino-4-methyl-cyclohexyl)imidazole;
5-[4-(2-amino)pyrimidinyl]-4-(4-fluorophenyl)-1-(4-hydroxy-4-methyl-cyclohexyl)imidazole;
5-[4-(2-amino)pyrimidinyl]-4-(4-fluorophenyl)-1-(4-oxiranylcyclohexyl)imidazole;
4-(fluorophenyl)-1-(methyl-4-piperidinyl)-5-(2-methysulfinyl-4-pyrimidinyl)imidazole;
4-(fluorophenyl)-1-(methyl-4-piperidinyl)-5-(2-methylthio-4-pyrimidinyl)imidazole;
5-[(2-benzylamino)pyrimidin-4-yl]-4-(4-fluorophenyl)-1-(1-methylpiperidin-4-yl)imidazole;
4-(4-fluorophenyl)-1-(1-methylpiperidin-4-yl)-5-[2-(4-tetrahydrothiopyranyl)aminopyrimidin-4-yl]imidazole;
4-(4-fluorophenyl)-5-[(2-hydroxy)ethylamino]pyrimidin-4-yl-1-(1-methylpiperidin-4-yl)imidazole;
5-[(2-(3-chlorobenzylamino)pyrimidin-4-yl]-4-(4-fluorophenyl)-1-(1-methylpiperidin-4-yl)imidazole;
5-[(2-(1-naphthylmethylamino)pyrimidin-4-yl]-4-(4-fluorophenyl)-1-(1-methylpiperidin-4-yl)imidazole;
5-[(2-(1-benzyl-4-piperidinylamino)pyrimidin-4-yl]-4-(4-fluorophenyl)-1-(1-methylpiperidin-4-yl)imidazole;
4-(4-fluorophenyl)-1-(1-methylpiperidin-4-yl)-5-[2-[3-(morpholino)propyl]aminopyrimidin-4-yl]imidazole;
5-[2-[(3-bromophenyl)amino]pyrimidin-4-yl]-4-(4-fluorophenyl)-1-(1-methylpiperidin-4-yl)imidazole;
5-[(2-(piperonylamino)pyrimidin-4-yl]-4-(4-fluorophenyl)-1-(1-methylpiperidin-4-yl)imidazole;
5-[(2-(4-piperidinylamino)pyrimidin-4-yl]-4-(4-fluorophenyl)-1-(1-methylpiperidin-4-yl)imidazole;
5-[(2-(5-chlorotryptamino)pyrimidin-4-yl]-4-(4-fluorophenyl)-1-(1-methylpiperidin-4-yl)imidazole;
5-[(2-(2,2,6,6-tetramethylpiperidin-4-yl)aminopyrimidin-4-yl]-4-(4-fluorophenyl)-1-(1-methylpiperidin-4-yl)imidazole;
5-[(2-[1-ethoxycarbonyl)piperidin-4-yl]aminopyrimidin-4-yl]-4-(4-fluorophenyl)-1-(1-methylpiperidin-4-yl)imidazole;
1-(4-oxocyclohexyl)-4-(4-fluorophenyl)-5-[(2-methoxy)pyrimidin-4-yl]imidazole;
cis-1-(4-hydroxycyclohexyl)-4-(4-fluorophenyl)-5-[(2-methoxy)pyrimidin-4-yl]imidazole;
trans-1-(4-hydroxycyclohexyl)-4-(4-fluorophenyl)-5-[(2-methoxy)pyrimidin-4-yl]imidazole;
1-(4-oxocyclohexyl)-4-(4-fluorophenyl)-5-[(2-methylthio)pyrimidin-4-yl]imidazole;
trans-1-(4-hydroxycyclohexyl)-4-(4-fluorophenyl)-5-[(2-methylthio)pyrimidin-4-yl]imidazole;
1-(4-oxocyclohexyl)-4-(4-fluorophenyl)-5-[(2-hydroxy)pyrimidin-4-yl]imidazole;
1-(4-oxocyclohexyl)-4-(4-fluorophenyl)-5-[(2-isopropoxy)pyrimidin-4-yl]imidazole;
1-(4-hydroxycyclohexyl)-4-(4-fluorophenyl)-5-[(2-isopropoxy)pyrimidin-4-yl]imidazole;
trans-1-(4-hydroxy-4-methylcyclohexyl)-4-(4-fluorophenyl)-5-[(2-methoxy)pyrimidin-4-yl]imidazole;
cis-1-(4-hydroxy-4-methylcyclohexyl)-4-(4-fluorophenyl)-5-[(2-methoxy)pyrimidin-4-yl]imidazole;
trans-1-(4-hydroxycyclohexyl)-4-(4-fluorophenyl)-5-[(2-ethoxy)pyrimidin-4-yl]imidazole;
1-(4-piperidinyl)-4-(4-fluorophenyl)-5-(2-phenoxypyrimidin-4-yl)imidazole;
1-(4-piperidinyl)-4-(4-fluorophenyl)-5-(2-phenoxy-4-pyridinyl)imidazole;
1-(4-piperidinyl)-4-(4-fluorophenyl)-5-[2-(4-methoxyphenoxy)-4-pyridinyl]imidazole;
1-(4-piperidinyl)-4-(4-fluorophenyl)-5-[2-(4-fluorophenoxy)-4-pyridinyl]imidazole;
1-(piperidin-4-yl)-4-(4-fluorophenyl)-5-[2-(4-methoxyphenoxy)pyrimidin-4-yl]imidazole;
1-(piperidin-4-yl)-4-(4-fluorophenyl)-5-[2-(4-fluorophenoxy)pyrimidin-4-yl]imidazole;
1-(piperidin-4-yl)-4-(4-fluorophenyl)-5-[2-(4-aminocarbonylphenoxy)pyrimidin-4-yl]imidazole;
1-(piperidin-4-yl)-4-(4-fluorophenyl)-5-[2-(4-ethylphenoxy)pyrimidin-4-yl]imidazole;
1-(piperidin-4-yl)-4-(4-fluorophenyl)-5-[2-(4-benzyloxyphenoxy)pyrimidin-4-yl]imidazole;
1-(piperidin-4-yl)-4-(4-fluorophenyl)-5-[2-(4-cyanophenoxy)pyrimidin-4-yl]imidazole;
1-(piperidin-4-yl)-4-(4-fluorophenyl)-5-[2-(4-hydroxyphenoxy)pyrimidin-4-yl]imidazole;
1-(4-hydroxycyclohexyl)-4-(4-fluorophenyl)-5-[2-(phenoxy)pyrimidin-4-yl]imidazole;
1-(piperidin-4-yl)-4-(4-fluorophenyl)-5-[2-(2,6-dimethylphenoxy)pyridin-4-yl]imidazole;
1-(piperidin-4-yl)-4-(4-fluorophenyl)-5-[2-(4-methylphenoxy)pyridin-4-yl]imidazole;
1-(piperidin-4-yl)-4-(4-fluorophenyl)-5-[2-(4-chlorophenoxy)pyridin-4-yl]imidazole;
1-[3-(N-morpholino)propyl]-4-(4-fluorophenyl)-5-[2-(phenoxy)pyrimidin-4-yl]imidazole;
1-(piperidin-4-yl)-4-(4-fluorophenyl)-5-[2-(3-methoxyphenoxy)pyrimidin-4-yl]imidazole;
1-(piperidin-4-yl)-4-(4-fluorophenyl)-5-[2-(4-phenylphenoxy)pyrimidin-4-yl]imidazole;
1-(piperidin-4-yl)-4-(4-fluorophenyl)-5-[2-(4-phenoxyphenoxy)pyrimidin-4-yl]imidazole;
1-(piperidin-4-yl)-4-(4-fluorophenyl)-5-[2-(3-hydroxyphenoxy)pyrimidin-4-yl]imidazole;
1-(3-(N-morpholino)propyl)-4-(4-fluorophenyl)-5-[2-(4-fluorophenoxy)pyrimidin-4-yl]imidazole;
1-(piperidin-4-yl)-4-(4-fluorophenyl)-5-[2-(2-hydroxyphenoxy)pyrimidin-4-yl]imidazole;
1-(piperidin-4-yl)-4-(4-fluorophenyl)-5-[2-((3,4-methylenedioxy)phenoxy)pyrimidin-4-yl]imidazole;
1-(piperidin-4-yl)-4-(4-fluorophenyl)-5-[2-(3-fluorophenoxy)pyrimidin-4-yl]imidazole;
1-(piperidin-4-yl)-4-(4-fluorophenyl)-5-[2-(2-fluorophenoxy)pyrimidin-4-yl]imidazole;
1-(piperidin-4-yl)-4-(4-fluorophenyl)-5-[2-(2-methoxyphenoxy)pyrimidin-4-yl]imidazole;
1-(piperidin-4-yl)-4-(4-fluorophenyl)-5-[2-(3-trifluoromethylphenoxy)pyrimidin-4-yl]imidazole;
1-(piperidin-4-yl)-4-(4-fluorophenyl)-5-[2-(3,4-difluorophenoxy)pyrimidin-4-yl]imidazole;
1-(piperidin-4-yl)-4-(4-fluorophenyl)-5-[2-(4-methylsulfonylphenoxy)pyrimidin-4-yl]imidazole;
1-(4-piperidinyl)-4-(4-fluorophenyl)-5-(2-thiophenoxypyrimidin-4yl)imidazole;
1-(4-piperidinyl)-4-(4-fluorophenyl)-5-[2-(1-methyltetrazol-5-ylthio)pyridin-4-yl]imidazole;
5-[2-(2-hydroxyethoxy)pyrimidin-4-yl]-4-(4-fluorophenyl)-1-(4-oxocyclohexyl)imidazole;
5-[2-(2-hydroxyethoxy)]pyrimidin-4-yl)-4-(4-fluorophenyl)-1-(4-hydroxycyclohexyl)imidazole;
5-[2-(2-tert-butylamino)ethoxypyrimidin-4-yl]-4-(4-fluorophenyl)-1-(4-oxocyclohexyl)imidazole;
5-[2-(2-tert-butylamino)ethoxypyrimidin-4-yl]-4-(4-fluorophenyl)-1-(4-hydroxycyclohexyl)imidazole;
1-(4-piperidinyl)-4-(4-Fluorophenyl)-5-(2-isopropoxy-4-pyrimidinyl)imidazole;
1-(4-piperidinyl)-4-(4-Fluorophenyl)-5-(2-methoxy-4-pyrimidinyl)imidazole;
5-(2-hydroxy-4-pyrimidinyl)-4-(4-fluorophenyl)-1-(4-piperidinyl)imidazole;
5-(2-methoxy-4-pyridinyl)-4-(4-fluorophenyl)-1-(4-piperidinyl)imidazole;
5-(2-isopropoxy-4-pyridinyl)-4-(4-fluorophenyl)-1-(4-piperidinyl)imidazole;
5-(2-methylthio-4-pyrimidinyl)-4-(4-fluorophenyl)-1-(4-piperidinyl)imidazole;
5-(2-methylthio-4-pyrimidinyl)-4-(4-fluorophenyl)-1-[1-methyl-4-piperidinyl]imidazole;
5-(2-ethoxy-4-pyrimidinyl)-4-(4-fluorophenyl)-1-(4-piperidinyl)imidazole;
1-(1-ethylcarboxylpiperidin-4-yl)-3-(4-thiomethylphenyl)-5-[2-(thiomethyl)pyrimidin-4-yl]-imidazole;
1-(1-ethylcarbonylpiperidin-4-yl)-4-(4-methylsulfinylphenyl)-5-[(2-methylsulfinyl)pyrimidin-4-yl]imidazole;
2-(4-methylthiophenyl)-4-(4-fluorophenyl)-5-(2-methoxy-4-pyrimidinyl)imidazole;
2-(4-methylsulfinylphenyl)-4-(4-fluorophenyl)-5-(2-methoxy-4-pyrimidinyl)imidazole;
2-[(4-N,N-dimethyl)aminomethylphenyl]-4-(4-fluorophenyl)-5-(2-methoxy-4-pyrimidinyl)imidazole;
2-[(4-N,N-dimethyl)aminomethylphenyl]-4-(4-fluorophenyl)-5-(2-phenoxy-4-pyrimidinyl)imidazole;
(+/−)-2-(4-methylsulfinylphenyl]-4-(4-fluorophenyl)-5-(2-phenoxy-4-pyrimidinyl)imidazole;
2-(4-methylthiophenyl]-4-(4-fluorophenyl)-5-(2-phenoxy-4-pyrimidinyl)imidazole;
   and pharmaceutically acceptable salts thereof.

Compounds useful in the practice of the present invention also include, but are not limited to, compounds of formula: 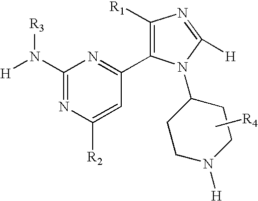
wherein

R₁ is hydrogen, C₁₋₅ alkyl, halogen, C₁₋₅ alkoxy, or arylC₁₋₅ alkyl;

R₂ and R₄ are independently hydrogen, C₁₋₅ alkyl, aryl, arylC₁₋₅ alkyl, heteroaryl, heteroarylC₁₋₅ alkyl, heterocyclic, or heterocyclicC₁₋₅ alkyl; and

R₃ is hydrogen or C₁₋₃ alkyl;

or a pharmaceutically-acceptable salt thereof.

Compounds useful in the practice of the present invention also include, but are not limited to, compounds of formula: 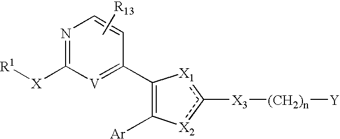
wherein

X is O, CH₂, S or NH, or the moiety X—R¹ is hydrogen;

R¹ is hydrogen, C₁₋₆ alkyl, C₃₋₇ cycloalkyl, aryl, arylC₁₋₆ alkyl, heterocyclyl, heterocyclylC₁₋₆ alkyl, heteroaryl, or heteroarylC₁₋₆ alkyl, any of which, except for hydrogen, can be optionally substituted;

V is CH or N;

Ar is an aryl or heteroaryl ring, either of which can be optionally substituted; one of X₁ and X₂ is N, and the other is NR¹⁵, wherein R¹⁵ is hydrogen, C₁₋₆ alkyl, or arylC₁₋₆ alkyl;

X₃ is a covalent bond or C(R²)(R³);

R² and R³ independently represent optionally substituted C₁₋₆ alkyl, or R² and R³ together with the carbon atom to which they are attached form an optionally substituted C₃₋₇ cycloalkyl, C₃₋₇ cycloalkenyl, or 5- to 7-membered heterocyclyl ring containing up to three heteroatoms independently selected from N, O, and S;

n is 0, 1, 2, 3, or 4;

Y is NR¹⁰R¹¹, NR¹⁰C(Z)NR¹⁰R¹¹, NR¹⁰COOR¹¹, NR¹⁰SO₂R¹¹, or C(O)NR⁴R⁵;

R⁴ and R⁵ independently represent hydrogen, C₁₋₆ alkyl, C₃₋₇ cycloalkyl, aryl, arylC₁₋₆ alkyl, heteroaryl, heteroarylC₁₋₆ alkyl, heterocyclyl, or heterocyclylC₁₋₆ alkyl, any one of which, except hydrogen, can be optionally substituted, or R⁴ and R⁵ together with the nitrogen atom to which they are attached form a 4- to 10-membered optionally-substituted monocyclic or bicyclic ring;

R¹³ is hydrogen, X—R¹, halogen, optionally-substituted C₁₋₆ alkylsulfinyl, CH₂OR¹⁴, di-C₁₋₆ alkylamino, N(R⁶)C(O)R⁷, N(R⁶)S(O)₂R⁸, or a 5- to 7-membered N-heterocyclyl ring which optionally contains an additional heteroatom selected from O, S, and NR⁹;

R¹⁴ is hydrogen, —C(Z)R¹² or optionally-substituted C₁₋₆ alkyl, optionally-substituted aryl, optionally-substituted arylC₁₋₆ alkyl or S(O)₂R⁸;

R⁶ is hydrogen or C₁₋₆ alkyl;

R⁷ is hydrogen, C₁₋₆ alkyl, C₃₋₇ cycloalkyl, aryl, arylC₁₋₆ alkyl, heteroaryl, heteroarylC₁₋₆ alkyl, heterocyclyl or heterocyclylC₁₋₆ alkyl;

R⁸ is C₁₋₆ alkyl, C₃₋₇ cycloalkyl, aryl, arylC₁₋₆ alkyl, heteroaryl, heteroarylC₁₋₆ alkyl, heterocyclyl or heterocyclylC₁₋₆ alkyl;

R⁹ is hydrogen, cyano, C₁₋₄ alkyl, C₃₋₇ cycloalkyl or aryl;

R¹⁰, R¹¹ and R¹² are independently selected from hydrogen, C₁₋₆ alkyl, C₃₋₇ cycloalkyl, heterocyclyl, heterocyclylC₁₋₆ alkyl, heterocyclylC₂₋₆ alkenyl, aryl, arylC₁₋₆ alkyl, arylC₂₋₆ alkenyl, heteroaryl, heteroarylC₁₋₆ alkyl and heteroarylC₂₋₆ alkenyl, any of which can be optionally substituted; or NR¹⁰R¹¹ can represent a 5- to 7-membered heterocyclyl ring optionally containing an additional heteroatom selected from O, N and S; and

Z is oxygen or sulfur;

or a pharmaceutically-acceptable salt thereof.

Compounds useful in the practice of the present invention also include, but are not limited to, compounds of formulas: 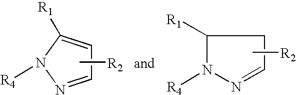
wherein

R₁ is a heteroaryl selected from 4-pyridyl, 4-pyrimidinyl, 4-quinolyl, 6-isoquinolinyl, quinazolin-4-yl, 1-imidazolyl, 1-benzimidazolyl, 4-pyridazinyl, and a 1,2,4-triazin-5-yl ring, which heteroaryl ring is substituted one to three times with Y, NHRₐ, optionally-substituted C₁₋₄ alkyl, halogen, hydroxyl, optionally-substituted C₁₋₄ alkoxy, optionally-substituted C₁₋₄ alkylthio, optionally-substituted C₁₋₄ alkylsulfinyl, CH₂OR₁₂, amino, mono- and di-C₁₋₆ alkyl-substituted amino, N(R₁₀)C(O)R_{b}, N(R₁₀)S(O)₂R_{d}, or an N-heterocyclyl ring which has from 5 to 7 members and optionally contains an additional heteroatom selected from oxygen, sulfur or NR₁₅;

Y is X₁—Rₐ;

X₁ is oxygen or sulfur;

Rₐ is C₁₋₆ alkyl, aryl, arylC₁₋₆ alkyl, heterocyclic, heterocyclylC₁₋₆ alkyl, heteroaryl, or heteroarylC₁₋₆ alkyl, wherein each of these moieties can be optionally substituted;

R_{b} is hydrogen, C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl;

R_{d} is C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl;

R₄ is phenyl, naphth-1-yl, naphth-2-yl, a heteroaryl or a fused phenyl-containing ring system, which is optionally substituted by one or two substituents, each of which is independently selected, and which, for a 4-phenyl, 4-naphth-1-yl, 5-naphth-2-yl or 6-naphth-2-yl substituent, is halogen, cyano, nitro, —C(Z)NR₇R₁₇, —C(Z)OR₁₆, —(CR₁₀R₂₀)ᵥCOR₁₂, —SR₅, —SOR₅, —OR₁₂, halo-substituted-C₁₋₄ alkyl, C₁₋₄ alkyl, -ZC(Z)R₁₂, —NR₁₀C(Z)R₁₆, or —(CR₁₀R₂₀)ᵥNR₁₀R₂₀ and which, for other positions of substitution, is halogen, cyano, nitro, phenyl, —C(Z)NR₁₃R₁₄, —C(Z)OR_{f}, —(CR₁₀R₂₀)_{m″}COR_{f}, —S(O)ₘR_{f}, —OR_{f}, halo-substituted C₁₋₄ alkyl, C₁₋₁₀ alkyl, -ZC(Z)R_{f}, optionally-substituted phenyl, —(CR₁₀R₂₀)_{m″}NR₁₀C(Z)R_{f}, —NR₁₀S(O)_{m′}R₈, —NR₁₀S(O)_{m′}NR₇R₁₇, -ZC(Z)R₁₂, or —(CR₁₀R₂₀)_{m″}R₁₃R₁₄;

R_{f} is heterocyclyl, heterocyclylC₁₋₁₀ alkyl or R₈;

v is 0, 1, or 2;

m is 0, 1, or 2;

m′ is 1 or 2;

m″ is 0, 1, 2, 3, 4, or 5;

R₂ hydrogen, —(CR₁₀R₂₃)ₙOR₉, heterocylyl, heterocyclylC₁₋₁₀ alkyl, C₁₋₁₀ alkyl, halo-substituted C₁₋₁₀ alkyl, C₂₋₁₀ alkenyl, C₂₋₁₀ alkynyl, C₃₋₇ cycloalkyl, C₃₋₇ cycloalkylC₁₋₁₀ alkyl, C₅₋₇ cycloalkenyl, C₅₋₇cycloalkenylC₁₋₁₀ alkyl, aryl, arylC₁₋₁₀ alkyl, heteroaryl, heteroarylC₁₋₁₀ alkyl, (CR₁₀R₂₃)ₙOR₁₁, (CR₁₀R₂₃)ₙS(O)ₘR₁₈, (CR₁₀R₂₃)ₙNHS(O)₂R₁₈, (CR₁₀R₂₃)ₙNR₁₃R₁₄, (CR₁₀R₂₃)ₙNO₂, (CR₁₀R₂₃)ₙCN, (CR₁₀R₂₃)ₙS(O)_{m′}NR₁₃R₁₄, (CR₁₀R₂₃)ₙC(Z)R₁₁, (CR₁₀R₂₃)ₙOC(Z)R₁₁, (CR₁₀R₂₃)ₙC(Z)OR₁₁, (CR₁₀R₂₃)ₙC(Z)NR₁₃R₁₄, (CR₁₀R₂₃)ₙC(Z)NR₁₁OR₉, (CR₁₀R₂₃)ₙNR₁₀C(Z)R₁₁, (CR₁₀R₂₃)ₙNR₁₀C(Z)NR₁₃R₁₄, (CR₁₀R₂₃)ₙN(OR₆)C(Z)NR₁₃R₁₄, (CR₁₀R₂₃)ₙN(OR₆)C(Z)R₁₁, (CR₁₀R₂₃)ₙC(═NOR₆)R₁₁, (CR₁₀R₂₃)ₙNR₁₀C(═NR₁₉)NR₁₃R₁₄, (CR₁₀R₂₃)ₙOC(Z)NR₁₃R₁₄, (CR₁₀R₂₃)ₙNR₁₀C(Z)NR₁₃R₁₄, (CR₁₀R₂₃)ₙNR₁₀C(Z)OR₁₀, 5-(R₁₈)-1,2,4-oxadiazol-3-yl or 4-(R₁₂)-5-(R₁₈R₁₉)-4,5-dihydro-1,2,4-oxadiazol-3-yl; wherein the aryl, arylalkyl, heteroaryl, heteroaryl alkyl, cycloalkyl, cycloalkyl alkyl, heterocyclic and heterocyclic alkyl groups can be optionally substituted;

n is 0, or an integer having a value of 1 to 10;

Z is oxygen or sulfur;

R₅ is hydrogen, C₁₋₄ alkyl, C₂₋₄ alkenyl, C₂₋₄ alkynyl or NR₇R₁₇, excluding the moieties —SR₅ being —SNR₇R₁₇ and —S(O)R₅ being —SOH;

R₆ is hydrogen, a pharmaceutically-acceptable cation, C₁₋₁₀ alkyl, C₃₋₇ cycloalkyl, aryl, arylC₁₋₄ alkyl, heteroaryl, heteroarylC₁₋₄ alkyl, heterocyclyl, aroyl, or C₁₋₁₀ alkanoyl;

R₇ and R₁₇ are each independently selected from hydrogen or C₁₋₄ alkyl, or R₇ and R₁₇ together with the nitrogen to which they are attached form a heterocyclic ring of 5 to 7 members which ring optionally contains an additional heteroatom selected from oxygen, sulfur or NR₁₅;

R₈ is C₁₋₁₀ alkyl, halo-substituted C₁₋₁₀ alkyl, C₂₋₁₀ alkenyl, C₂₋₁₀ alkynyl, C₃₋₇ cycloalkyl, C₅₋₇ cycloalkenyl, aryl, arylC₁₋₁₀ alkyl, heteroaryl, heteroarylC₁₋₁₀ alkyl, (CR₁₀R₂₀)ₙOR₁₁, (CR₁₀R₂₀)ₙS(O)ₘR₁₈, (CR₁₀R₂₀)ₙNHS(O)₂R₁₈, or (CR₁₀R₂₀)ₙNR₁₃R₁₄, wherein the aryl, arylalkyl, heteroaryl, and heteroaryl alkyl can be optionally substituted;

R₉ is hydrogen, —C(Z)R₁₁, optionally-substituted C₁₋₁₀ alkyl, S(O)₂R₁₈, optionally-substituted aryl or optionally-substituted arylC₁₋₄ alkyl;

R₁₀ and R₂₀ are each independently selected from hydrogen or C₁₋₄ alkyl;

R₁₁ is hydrogen, C₁₋₁₀ alkyl, C₃₋₇ cycloalkyl, heterocyclyl, heterocyclylC₁₋₁₀ alkyl, aryl, arylC₁₋₁₀ alkyl, heteroaryl or heteroarylC₁₋₁₀ alkyl, wherein the aryl, arylalkyl, heteroaryl, heteroaryl alkyl, heterocyclyl or heterocyclylalkyl can be optionally substituted;

R₁₂ is hydrogen or R₁₆;

R₁₃ and R₁₄ are each independently selected from hydrogen or optionally-substituted C₁₋₄ alkyl, optionally-substituted aryl or optionally-substituted arylC₁₋₄ alkyl, or together with the nitrogen to which they are attached form a heterocyclic ring of 5 to 7 members which ring optionally contains an additional heteroatom selected from oxygen, sulfur or NR₉;

R₁₅ is hydrogen, C₁₋₄ alkyl or C(Z)-C₁₋₄ alkyl;

R₁₆ is C₁₋₄ alkyl, halo-substituted C₁₋₄ alkyl, or C₃₋₇ cycloalkyl;

R₁₈ is C₁₋₁₀ alkyl, C₃₋₇ cycloalkyl, heterocyclyl, aryl, arylC₁₋₁₀ alkyl, heterocyclyl, heterocyclylC₁₋₁₀ alkyl, heteroaryl or heteroarylC₁₋₁₀ alkyl, wherein the aryl, arylalkyl, heteroaryl, heteroaryl alkyl, heterocyclyl or heterocyclylalkyl can be optionally substituted;

R₁₉ is hydrogen, cyano, C₁₋₄ alkyl, C₃₋₇ cycloalkyl or aryl; and

R₂₃ is hydrogen, C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl, all of which can be optionally substituted;

or a pharmaceutically-acceptable salt thereof.

Exemplary compounds of these formulas include:
4-[1-(4-fluorophenyl)-3-phenyl-1H-pyrazol-5-yl]pyridine
4-[4-bromo-1-(4-fluorophenyl)-3-phenyl-1H-pyrazol-5-yl]pyridine
4-[1-(4-fluorophenyl)-3-[4-(methylthio)phenyl]-1H-pyrazol-5-yl]pyridine
4-[1-(4-fluorophenyl)-3-[4-(methylsulfonyl)phenyl]-1H-pyrazol-5-yl]pyridine 4-[1-(4-fluorophenyl)-3-[4-(methylsulfinyl)phenyl]-1H-pyrazol-5-yl]pyridine;
4-[1-(4-fluorophenyl)-4,5-dihydro-3-phenyl-1H-pyrazol-5-yl]pyridine
4-[1-(4-fluorophenyl)-4,5-dihydro-3-[4-(methylthio)phenyl]-1H-pyrazol-5-yl]pyridine
   and pharmaceutically acceptable salts thereof.

Compounds useful in the practice of the present invention also include, but are not limited to, compounds of formulas: 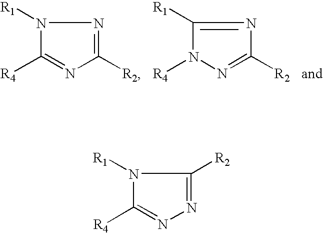
wherein

R₁ is 4-pyridyl or 4-pyrimidinyl ring, which ring is optionally substituted one or more times with Y, C₁₋₄ alkyl, halogen, hydroxyl, C₁₋₄ alkoxy, C₁₋₄ alkylthio, C₁₋₄ alkylsulfinyl, CH₂OR₁₂, amino, mono- and di-C₁₋₆ alkyl-substituted amino, N(R₁₀)C(O)R_{b}, or an N-heterocyclyl ring which has from 5 to 7 members and optionally contains an additional heteroatom selected from oxygen, sulfur or NR₁₅;

Y is X₁—Rₐ;

X₁ is oxygen, sulfur, or NH;

Rₐ is C₁₋₆ alkyl, aryl, arylC₁₋₆ alkyl, heterocyclic, heterocyclylC₁₋₆ alkyl, heteroaryl, or heteroarylC₁₋₆ alkyl, wherein each of these moieties can be optionally substituted;

R_{b} is hydrogen, C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl, wherein each of these moieties can be optionally substituted;

R₄ is phenyl, naphth-1-yl, naphth-2-yl, or a heteroaryl, which is optionally substituted by one or two substituents, each of which is independently selected, and which, for a 4-phenyl, 4-naphth-1-yl, 5-naphth-2-yl or 6-naphth-2-yl substituent, is halogen, cyano, nitro, —C(Z)NR₇R₁₇, —C(Z)OR₁₆, —(CR₁₀R₂₀)ᵥCOR₁₂, —SR₅, —SOR₅, —OR₁₂, halo-substituted-C₁₋₄ alkyl, C₁₋₄ alkyl, -ZC(Z)R₁₂, —NR₁₀C(Z)R₁₆, or —(CR₁₀R₂₀)ᵥNR₁₀R₂₀ and which, for other positions of substitution, is halogen, cyano, —C(Z)NR₁₃R₁₄, —C(Z)OR_{f}, —(CR₁₀R₂₀)_{m″}COR_{f}, —S(O)_{m″}R_{f}, —OR_{f}, halo-substituted C₁₋₄ alkyl, C₁₋₄ alkyl, -ZC(Z)R_{f}, —(CR₁₀R₂₀)_{m″}NR₁₀C(Z)R_{f}, —NR₁₀S(O)_{m′}R₈, —NR₁₀S(O)_{m′}NR₇R₁₇, or —(CR₁₀R₂₀)_{m″}NR₁₃R₁₄;

R_{f} is heterocyclyl, heterocyclylC₁₋₁₀ alkyl or R₈;

v is 0, 1, or 2;

m is 0, 1, or 2;

m′ is 1 or 2;

m″ is 0, 1, 2, 3, 4, or 5;

R₂ hydrogen, C(HOURS)(A)(R₂₂), —(CR₁₀R₂₃)ₙOR₉, heterocylyl, heterocyclylC₁₋₁₀ alkyl, C₁₋₁₀ alkyl, halo-substituted C₁₋₁₀ alkyl, C₂₋₁₀ alkenyl, C₂₋₁₀ alkynyl, C₃₋₇ cycloalkyl, C₃₋₇ cycloalkylC₁₋₁₀ alkyl, C₅₋₇ cycloalkenyl, C₅₋₇cycloalkenylC₁₋₁₀ alkyl, aryl, arylC₁₋₁₀ alkyl, heteroaryl, heteroarylC₁₋₁₀ alkyl, (CR₁₀R₂₃)ₙOR₁₁, (CR₁₀R₂₃)ₙS(O)ₘR₁₈, (CR₁₀R₂₃)ₙNHS(O)₂R₁₈, (CR₁₀R₂₃)ₙNR₁₃R₁₄, (CR₁₀R₂₃)ₙNO₂, (CR₁₀R₂₃)ₙCN, (CR₁₀R₂₃)ₙS(O)_{m′}NR₁₃R₁₄, (CR₁₀R₂₃)ₙC(Z)R₁₁, (CR₁₀R₂₃)ₙOC(Z)R₁₁, (CR₁₀R₂₃)ₙC(Z)OR₁₁, (CR₁₀R₂₃)ₙC(Z)NR₁₃R₁₄, (CR₁₀R₂₃)ₙC(Z)NR₁₁OR₉, (CR₁₀R₂₃)ₙNR₁₀C(Z)R₁₁, (CR₁₀R₂₃)ₙNR₁₀C(Z)NR₁₃R₁₄, (CR₁₀R₂₃)ₙN(OR₆)C(Z)NR₁₃R₁₄, (CR₁₀R₂₃)ₙN(OR₆)C(Z)R₁₁, (CR₁₀R₂₃)ₙC(═NOR₆)R₁₁, (CR₁₀R₂₃)ₙNR₁₀C(═NR₁₉)NR₁₃R₁₄, (CR₁₀R₂₃)ₙOC(Z)NR₁₃R₁₄, (CR₁₀R₂₃)ₙNR₁₀C(Z)NR₁₃R₁₄, (CR₁₀R₂₃)ₙNR₁₀C(Z)OR₁₀, 5-(R₁₈)-1,2,4-oxadiazol-3-yl or 4-(R₁₂)-5-(R₁₈R₁₉)-4,5-dihydro-1,2,4-oxadiazol-3-yl; wherein the aryl, arylalkyl, heteroaryl, heteroaryl alkyl, cycloalkyl, cycloalkyl alkyl, heterocyclic and heterocyclic alkyl groups can be optionally substituted;

A is an optionally-substituted aryl, heterocyclyl or heteroaryl ring, or A is a substituted C₁₋₁₀ alkyl;

n is 0, or an integer having a value of 1 to 10;

Z is oxygen or sulfur;

R₅ is hydrogen, C₁₋₄ alkyl, C₂₋₄ alkenyl, C₂₋₄ alkynyl or NR₇R₁₇, excluding the moieties —SR₅ being —SNR₇R₁₇ and —S(O)R₅ being —SOH;

R₆ is hydrogen, a pharmaceutically-acceptable cation, C₁₋₁₀ alkyl, C₃₋₇ cycloalkyl, aryl, arylC₁₋₄ alkyl, heteroaryl, heteroarylC₁₋₄ alkyl, heterocyclyl, aroyl, or C₁₋₁₀ alkanoyl;

R₇ and R₁₇ are each independently selected from hydrogen or C₁₋₄ alkyl, or R₇ and R₁₇ together with the nitrogen to which they are attached form a heterocyclic ring of 5 to 7 members which ring optionally contains an additional heteroatom selected from oxygen, sulfur or NR₁₅;

R₈ is C₁₋₁₀ alkyl, halo-substituted C₁₋₁₀ alkyl, C₂₋₁₀ alkenyl, C₂₋₁₀ alkynyl, C₃₋₇ cycloalkyl, C₅₋₇ cycloalkenyl, aryl, arylC₁₋₁₀ alkyl, heteroaryl, heteroarylC₁₋₁₀ alkyl, (CR₁₀R₂₀)ₙOR₁₁, (CR₁₀R₂₀)ₙS(O)ₘR₁₈, (CR₁₀R₂₀)ₙNHS(O)₂R₁₈, or (CR₁₀R₂₀)ₙNR₁₃R₁₄, wherein the aryl, arylalkyl, heteroaryl, and heteroaryl alkyl can be optionally substituted;

R₉ is hydrogen, —C(Z)R₁₁, optionally-substituted C₁₋₁₀ alkyl, S(O)₂R₁₈, optionally-substituted aryl or optionally-substituted arylC₁₋₄ alkyl;

R₁₀ and R₂₀ are each independently selected from hydrogen or C₁₋₄ alkyl;

R₁₁ is hydrogen, C₁₋₁₀ alkyl, C₃₋₇ cycloalkyl, heterocyclyl, heterocyclylC₁₋₁₀ alkyl, aryl, arylC₁₋₁₀ alkyl, heteroaryl or heteroarylC₁₋₁₀ alkyl, wherein the aryl, arylalkyl, heteroaryl, heteroaryl alkyl, heterocyclyl or heterocyclylalkyl can be optionally substituted;

R₁₂ is hydrogen or R₁₆;

R₁₃ and R₁₄ are each independently selected from hydrogen or optionally-substituted C₁₋₄ alkyl, optionally-substituted aryl or optionally-substituted arylC₁₋₄ alkyl, or together with the nitrogen to which they are attached form a heterocyclic ring of 5 to 7 members, which ring optionally contains an additional heteroatom selected from oxygen, sulfur or NR₉;

R₁₅ is R₁₀ or C(Z)C₁₋₄ alkyl;

R₁₆ is C₁₋₄ alkyl, halo-substituted C₁₋₄ alkyl, or C₃₋₇ cycloalkyl;

R₁₈ is C₁₋₁₀ alkyl, C₃₋₇ cycloalkyl, heterocyclyl, aryl, arylC₁₋₁₀ alkyl, heterocyclyl, heterocyclylC₁₋₁₀ alkyl, heteroaryl or heteroarylC₁₋₁₀ alkyl;

R₁₉ is hydrogen, cyano, C₁₋₄ alkyl, C₃₋₇ cycloalkyl or aryl; and

R₂₃ is hydrogen, C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl, all of which can be optionally substituted;

or a pharmaceutically-acceptable salt thereof.

Exemplary compounds of these formulas include:
1-(pyrid-4-yl)-3-phenyl-5-(4-fluorophenyl)-1,2,4-triazole;
1-(6-aminopyrimidin-4-yl)-3-phenyl-5-(4-fluorophenyl)-1,2,4-triazole;
1-[4-(6,7-dimethoxyquinazoline)]-3-phenyl-5-(4-fluorophenyl)-1,2,4-triazole;
1-(4-fluorophenyl)-3-phenyl-5-(2-aminopyrimidin-4-yl)-1,2,4-triazole;
3-(4-fluorophenyl)-4-(2-aminopyrimidin-4-yl)-5-phenyl-1,2,4-triazole;
   and pharmaceutically acceptable salts thereof.

Compounds useful in the practice of the present invention also include, but are not limited to, compounds of formula:

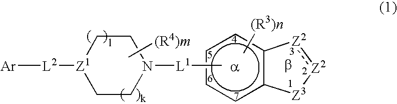

and the pharmaceutically acceptable salts thereof, or a pharmaceutical composition thereof, wherein

[symbol] represents a single or double bond;

one Z² is CA or CR⁸A and the other is CR¹, CR¹₂, NR⁶ or N wherein each R¹, R⁶ and R⁸ is independently hydrogen or noninterfering substituent;

A is —CO(X)ⱼY wherein Y is COR² or an isostere thereof and R² is hydrogen or a noninterfering substituent, X is a spacer preferably of 2-6 Å, and j is 0 or 1;

Z³ is NR⁷ or O;

each R³ is independently a noninterfering substituent;

n is 0-3;

each of L¹ and L² is a linker;

each R⁴ is independently a noninterfering substituent;

m is 0-4;

Z¹ is CR⁵ or N wherein R⁵ is hydrogen or a noninterfering substituent;

each of l and k is an integer from 0-2 wherein the sum of l and k is 0-3;

Ar is an aryl group substituted with 0-5 noninterfering substituents, wherein two noninterfering substituents can form a fused ring; and the distance between the atom of Ar linked to L² and the center of the α ring is preferably less than 24 Å. In the description above, certain positions of the molecule are described as permitting “noninterfering substituents.” This terminology is used because the substituents in these positions generally speaking are not relevant to the essential activity of the molecule taken as a whole. A wide variety of substituents can be employed in these positions, and it is well within ordinary skill to determine whether any particular arbitrary substituent is or is not “noninterfering.”

Compounds useful in the practice of the present invention also include, but are not limited to, compounds of formulas: 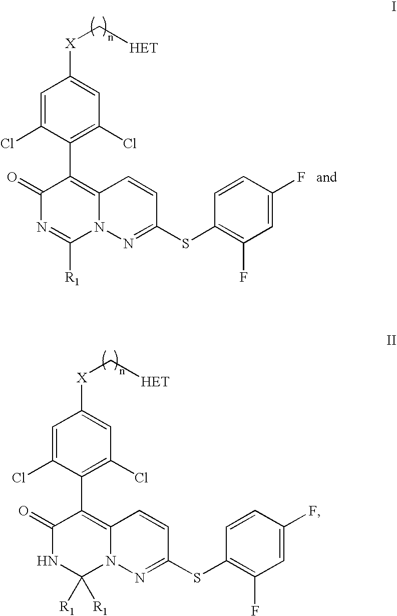
or

pharmaceutically acceptable salts thereof, wherein

HET is a 5-7 membered heterocycle with 1 to 4 N, S or O atoms, which heterocycle is substituted with 1 to 3 C₁-C₄ branched or straight chain alkyl groups. HET can optionally be substituted with halo, cyano, N(R′)₂, OR′, CO₂R′, CON(R′)₂, and SO₂N(R²)₂;

X is O or NR′;

n is 1 to 3;

R′ is selected from hydrogen, (C₁-C₃)-alkyl, (C₂-C₃)-alkenyl or alkynyl, phenyl or phenyl substituted with 1 to 3 substituents independently selected from halo, methoxy, cyano, nitro, amino, hydroxy, methyl or ethyl; or a 5-6 membered heterocyclic ring system optionally substituted with 1 to 3 substituents independently selected from halo, methoxy, cyano, nitro, amino, hydroxy, methyl or ethyl;

R₁ is selected from hydrogen, (C₁-C₃)-alkyl, hydroxy, or (C₁-C₃)-alkoxy;

R₂ is selected from hydrogen, (C₁-C₃)-alkyl, or (C₁-C₃)-alkenyloxy; each optionally substituted with —N(R′)₂, —OR′, —SR′, —C(O)—N(R′)₂, —S(O₂)—N(R′)₂, —C(O)—OR′, or R³; and

R³ is selected from 5-6 membered aromatic carbocyclic or heterocyclic ring systems.

Compounds useful in the practice of the present invention also include, but are not limited to, compounds of formulas: 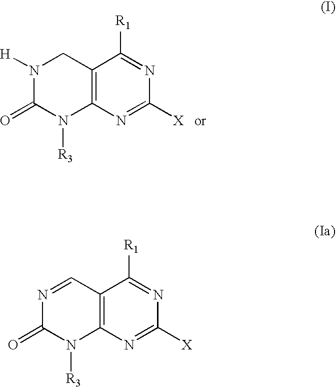
wherein

R₁ is an aryl or heteroaryl ring, which ring is optionally substituted;

R₂ is hydrogen, C₁₋₁₀ alkyl, C₃₋₇ cycloalkyl, C₃₋₇ cycloalkylC₁₋₁₀ alkyl, aryl, arylC₁₋₁₀ alkyl, heteroaryl, heteroarylC₁₋₁₀ alkyl, heterocyclic, or a heterocyclylC₁₋₁₀ alkyl moiety; and wherein each of these moieties, excluding hydrogen, are optionally substituted;

R₃ is a C₁₋₁₀ alkyl, C₃₋₇cycloalkyl, C₃₋₇cycloalkylC₁₋₁₀ alkyl, arylC₁₋₁₀alkyl, heteroaryl C₁₋₁₀alkyl, or heterocyclylC₁₋₁₀ alkyl moiety; and wherein each of these moieties are optionally substituted;

X is R₂, OR₂, S(O)ₘR₂ or (CH₂)ₙNR₄R₁₄, or (CH₂)ₙNR₂R₄;

n is 0 or an integer having a value of 1 to 10;

m is 0 or an integer having a value of 1 or 2;

R₄ and R₁₄ are each independently selected from hydrogen, optionally substituted C₁₋₁₄ alkyl, optionally substituted aryl, or an optionally substituted arylC₁₋₄alkyl, or R₄ and R₁₄ together with the nitrogen to which they are attached form a heterocyclic ring of 5 to 7 members, which ring optionally contains an additional heteroatom selected from oxygen, sulfur or NR₉, and which ring can be optionally substituted;

R₆ is hydrogen, C₁₋₁₀ alkyl, C₃₋₇ cycloalkyl, heterocyclyl, heterocyclylC₁₋₁₀alkyl, aryl, arylC₁₋₁₀ alkyl, heteroaryl or a heteroarylC₁₋₁₀ alkyl moiety; and wherein each of these moieties, excluding hydrogen, can be optionally substituted;

R₉ is hydrogen, C(Z)R₆, optionally substituted C₁₋₁₀ alkyl, optionally substituted aryl or optionally substituted arylC₁₋₄ alkyl;

Z is oxygen or sulfur;

or a pharmaceutically acceptable salt thereof.

Compounds useful in the practice of the present invention also include, but are not limited to, compounds of formulas: 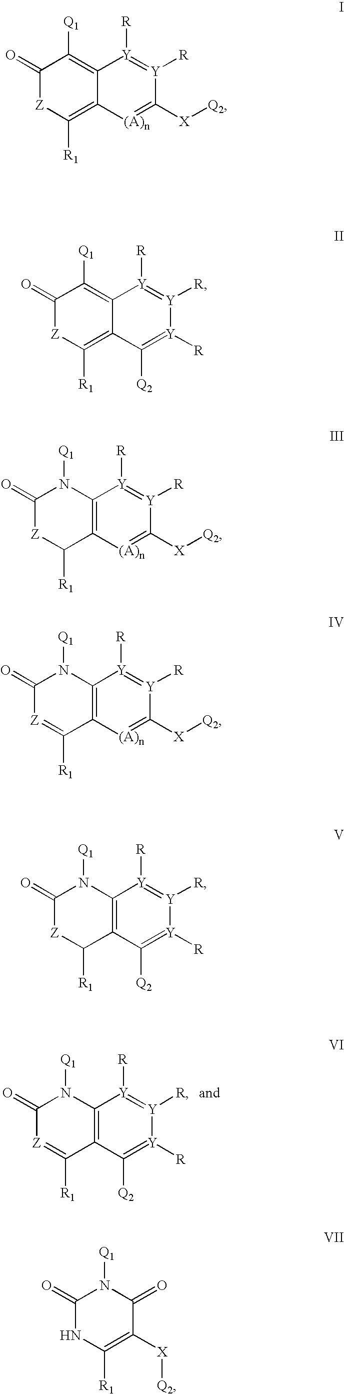
or pharmaceutically acceptable salts thereof, wherein

each of Q₁ and Q₂ are independently selected from 5-6 membered aromatic carbocyclic or heterocyclic ring systems, or 8-10 membered bicyclic ring systems comprising aromatic carbocyclic rings, aromatic heterocyclic rings or a combination of an aromatic carbocyclic ring and an aromatic heterocyclic ring; the rings that make up Q₁ are substituted with 1 to 4 substituents, each of which is independently selected from halo; C₁-C₃ alkyl optionally substituted with NR′₂, OR′, CO₂R′ or CONR′₂; (C₁-C₃)-alkoxy optionally substituted with NR′₂, OR′, CO₂R′ or CONR′₂; NR′₂; OCF₃; CF₃; NO₂; CO₂R′; CONR′; SR′; S(O₂)N(R′)₂; SCF₃; CN; N(R′)C(O)R⁴; N(R′)C(O)OR⁴; N(R′)C(O)C(O)R⁴; N(R′)S(O₂)R⁴; N(R′)R⁴; N(R⁴)₂; OR⁴; OC(O)R⁴; OP(O)₃H₂; or N═C—N(R′)₂; the rings that make up Q₂ are optionally substituted with up to 4 substituents, each of which is independently selected from halo; C₁-C₃ straight or branched alkyl optionally substituted with NR′₂, OR′, CO₂R′, S(O₂)N(R′)₂, N═C—N(R′)₂, R³, or CONR′₂; (C₁-C₃)-alkoxy optionally substituted with NR′₂, OR′, CO₂R′, S(O₂)N(R′)₂, N═C—N(R′)₂, R³, or CONR′₂; NR′₂, OCF₃; CF₃; NO₂; CO₂R′; CONR′; R³; OR³; NR³; SR³; C(O)R³; C(O)N(R′)R³; C(O)OR³; SR′; S(O₂)N(R′)₂; SCF₃; N═C—N(R′)₂; or CN;

R′ is selected from hydrogen, (C₁-C₃)-alkyl; (C₂-C₃)-alkenyl; (C₂-C₃) alkynyl; phenyl substituted with 1 to 3 substituents independently selected from halo, methoxy, cyano, nitro, amino, hydroxy, methyl or ethyl;

R³ is selected from 5-6 membered aromatic carbocyclic or heterocyclic ring systems;

R⁴ is (C₁-C₄)-alkyl optionally substituted with N(R′)₂, OR′, CO₂R′, CON(R′)₂, or SO₂N(R²)₂; or a 5-6 membered carbocyclic or heterocyclic ring system optionally substituted with N(R′)₂, OR′, CO₂R′, CON(R′)₂, or SO₂N(R²)₂;

X, if present, is selected from —S—, —O—, —S(O₂)—, —S(O)—, —S(O₂)—N(R²)—, —N(R²)—S(O₂)—, —N(R²)—C(O)O—, —O—C(O)—N(R²), —C(O)—, —C(O)O—, —O—C(O)—, —C(O)—N(R²)—, —N(R²)—C(O)—-N(R²)—, —C(R²)₂—, or —C(OR²)₂—;

each R is independently selected from hydrogen, —R², —N(R²)₂, —OR², SR², —C(O)—N(R²)₂, —S(O₂)—N(R²)₂, or —C(O)—OR², wherein two adjacent R are optionally bound to one another and, together with each Y to which they are respectively bound, form a 4-8 membered carbocyclic or heterocyclic ring;

R² is selected from hydrogen, (C₁-C₃)-alkyl, or (C₁-C₃)-alkenyl; each optionally substituted with —N(R′)₂, —OR′, SR′, —C(O)—N(R′)₂, —S(O₂)—N(R′)₂, —C(O)—OR′, or R³;

Y is N or C;

Z, if present, is N, NH, or, if chemically feasible, O;

A, if present, is N or CR′;

n is 0 or 1; and

R₁ is selected from hydrogen, (C₁-C₃)-alkyl, hydroxy, or (C₁-C₃)-alkoxy.

Compounds useful in the practice of the present invention also include, but are not limited to, compounds of formula: 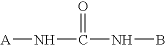
wherein A is 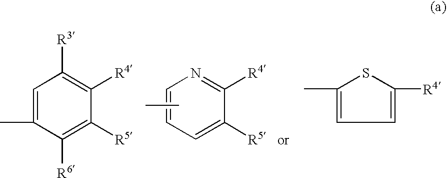
wherein

R^{3′}, R^{4′}, R^{5′} are each independently HOURS, C₁₋₁₀-alkyl, optionally substituted by halogen up to perhalo, C₁₋₁₀ alkoxy, optionally substituted by halogen, up to perhaloalkoxy, halogen; NO₂ or NH₂;

R^{6′} is HOURS, C₁₋₁₀-alkyl, C₁₋₁₀ alkoxy, —NHCOR¹; —NR¹COR¹; NO₂;

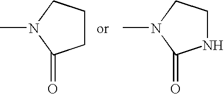

one of R^{4′}, R^{5′}, or R^{6′} can be —X—Y; or

2 adjacent R^{4′}-R^{6′} can together be an aryl or heteroaryl ring with 5-12 atoms, optionally substituted by C₁₋₁₀-alkyl, C₁₋₁₀ alkoxy, C₃₋₁₀ cycloalkyl, C₂₋₁₀ alkenyl, C₁₋₁₀ alkanoyl, C₆₋₁₂ aryl, C₅₋₁₂ heteroaryl or C₆₋₁₂ arakyl;

R¹ is C₁₋₁₀-alkyl optionally substituted by halogen, up to perhalo;

X is —CH₂—, —S—, —N(CH₃)—, —NHC(O)—, —CH₂—S—, —S—CH₂—, —C(O)—, or —O—;

X is additionally a single bond where Y is pyridyl;

Y is phenyl, pyridyl, naphthyl, pyridone, pyrazine, benzodioxane, benzopyridine, pyrimidine or benzothiazole, each optionally substituted by C₁₋₁₀-alkyl, C₁₋₁₀-alkoxy, halogen, OH, —SCH₃ or NO₂ or, where Y is phenyl, by 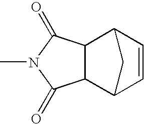
or a pharmaceutically-acceptable salt thereof;
or

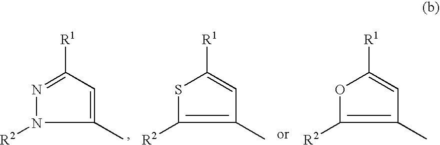

wherein

R¹ is selected from the group consisting of C₃-C₁₀ alkyl, C₃-C₁₀ cycloalkyl, up to per-halo substituted C₁-C₁₀ alkyl and up to per-halosubstituted C₃-C₁₀ cycloalkyl; and

R² is C₆-C₁₄ aryl, C₃-C₁₄ heteroaryl, substituted C₆-C₁₄ aryl or substituted C₃-C₁₄ heteroaryl;

wherein if R² is a substituted group, it is preferably substituted by one or more substituents independently selected from the group consisting of halogen, up to per-halosubstitution, and Vₙ, where n=0-3 and each V is independently selected from the group consisting of —CN, —OC(O)NR⁵R^{5′}, —CO₂R⁵, —C(O)NR⁵R^{5′}, —OR⁵, —SR⁵, —NR⁵R^{5′}, —C(O)R⁵, —NR⁵C(O)OR^{5′}, —SO₂R⁵—SOR⁵, —NR⁵C(O)R^{5′}, —NO₂, C₁-C₁₀ alkyl, C₃-C₁₀ cycloalkyl, C₆-C₁₄ aryl, C₃-C₁₃ heteroaryl, C₇-C₂₄ alkaryl, C₄-C₂₄ alkheteroaryl, substituted C₁-C₁₀ alkyl, substituted C₃-C₁₀ cycloalkyl, substituted C₆-C₁₄ aryl, substituted C₃-C₁₃ heteroaryl, substituted C₇-C₂₄ alkaryl and substituted C₄-C₂₄ alkheteroaryl;

wherein if V is a substituted group, it is substituted by one or more substituents independently selected from the group consisting of halogen, up to per-halosubstitution, —CN, —CO₂R⁵, —C(O)R⁵, —C(O)NR⁵R^{5′}, —NR⁵R^{5′}, —OR⁵, —SR⁵, —NR⁵C(O)R^{5′}, —NR⁵C(O)OR^{5′} and —NO₂; and

R⁵ and R^{5′} are independently selected form the group consisting of HOURS, C₁-C₁₀ alkyl, C₃-C₁₀ cycloalkyl, C₆-C₁₄ aryl, C₃-C₁₃ heteroaryl, C₇-C₂₄ alkaryl, C₄-C₂₃ alkheteroaryl, up to per-halosubstituted C₁-C₁₀ alkyl, up to per-halosubstituted C₃-C₁₀ cycloalkyl, up to per-halosubstituted C₆-C₁₄ aryl and up to per-halosubstituted C₃-C₁₃ heteroaryl;

or a pharmaceutically-acceptable salt thereof;

or

(c) a substituted moiety of up to 40 carbon atoms of the formula: -L-(M-L¹)_{q}, where L is a 5- or 6-membered cyclic structure bound directly to D, L¹, comprises a substituted cyclic moiety having at least 5 members, M is a bridging group having at least one atom, q is an integer of from 1-3; and each cyclic structure of L and L¹ contains 0-4 members of the group consisting of nitrogen, oxygen and sulfur;

L¹ is substituted by at least one substituent selected from the group consisting of —SO₂Rₓ, —C(O)Rₓ, and —C(NR_{y})R_{z};

R_{y} is hydrogen or a carbon-based moiety of up to 24 carbon atoms optionally containing heteroatoms selected from N, S and O and optionally halosubstituted, up to perhalo;

R_{z} is hydrogen or a carbon-based moiety of up to 30 carbon atoms optionally containing heteroatoms selected from N, S and O and optionally substituted by halogen, hydroxy and carbon-based substituents of up to 24 carbon atoms, which optionally contain heteroatoms selected from N, S and O and are optionally substituted by halogen; and

Rₓ is R_{z}, or NRₐR_{b} where Rₐ and R_{b} are

i) independently hydrogen,
a carbon-based moiety of up to 30 carbon atoms optionally containing heteroatoms selected from N, S and O and optionally substituted by halogen, hydroxy and carbon-based substituents of up to 24 carbon atoms, which optionally contain heteroatoms selected from N, S and O and are optionally substituted by halogen, or
—OSi(R_{f})₃ where R_{f} is hydrogen or a carbon-based moiety of up to 24 carbon atoms optionally containing heteroatoms selected from N, S and O and optionally substituted by halogen, hydroxy and carbon-based substituents of up to 24 carbon atoms, which optionally contain heteroatoms selected from N, S and O and are optionally substituted by halogen; or

ii) Rₐ and R_{b} together form a 5-7 member heterocyclic structure of 1-3 heteroatoms selected from N, S and O, or a substituted 5-7 member heterocyclic structure of 1-3 heteroatoms selected from N, S and O, substituted by halogen, hydroxy or carbon-based substituents of up to 24 carbon atoms, which optionally contain heteroatoms selected from N, S and O and are optionally substituted by halogen; or

iii) one of Rₐ or R_{b} is —C(O)—, a C₁-C₅ divalent alkylene group or a substituted C₁-C₅ divalent alkylene group bound to the moiety L to form a cyclic structure with at least 5 members, wherein the substituents of the substituted C₁-C₅ divalent alkylene group are selected from the group consisting of halogen, hydroxy, and carbon-based substituents of up to 24 carbon atoms, which optionally contain heteroatoms selected from N, S and O and are optionally substituted by halogen;

or a pharmaceutically-acceptable salt thereof; and

B is an unsubstituted or substituted, up to tricyclic, aryl or heteroaryl moiety with up to 30 carbon atoms with at least one 5- or 6-membered aromatic structure containing 0-4 members of the group consisting of nitrogen, oxygen and sulfur;

wherein if B is substituted, it is substituted by one or more substituents selected from the group consisting of halogen, up to per-halo, and Wₙ, wherein

n is 0-3 and each W is independently selected from the group consisting of

—CN, —CO₂R⁷, —C(O)NR⁷R⁷, —C(O)R⁷, —NO₂, —OR⁷, —SR⁷, —NR⁷R⁷,

—NR⁷C(O)OR⁷, —NR⁷C(O)R⁷, C₁-C₁₀ alkyl, C₂₋₁₀-alkenyl, C₁₋₁₀-alkoxy, C₃-C₁₀ cycloalkyl, C₆-C₁₄ aryl, C₇-C₂₄ alkaryl, C₃-C₁₃ heteroaryl, C₄-C₂₃ alkheteroaryl, substituted C₁-C₁₀ alkyl, substituted C₂₋₁₀-alkenyl, substituted C₁₋₁₀— alkoxy, substituted C₃-C₁₀ cycloalkyl, substituted C₄-C₂₃ alkheteroaryl and -Q-Ar;

wherein if W is a substituted group, it is substituted by one or more substituents independently selected from the group consisting of —CN, —CO₂R⁷,

—C(O)NR⁷R⁷, —C(O)R⁷, —NO₂, —OR⁷, —SR⁷, —NR⁷R⁷, —NR⁷C(O)OR⁷,

—NR⁷C(O)R⁷ and halogen up to per-halo;

wherein each R⁷ is independently selected from HOURS, C₁-C₁₀ alkyl, C₂₋₁₀-alkenyl, C₃-C₁₀ cycloalkyl, C₆-C₁₄ aryl, C₃-C₁₃ heteroaryl, C₇-C₂₄ alkaryl, C₄-C₂₃ alkheteroaryl, up to per-halosubstituted C₁-C₁₀ alkyl, up to per-halosubstituted C₂₋₁₀-alkenyl, up to per-halosubstituted C₃-C₁₀ cycloalkyl, up to per-halosubstituted C₆-C₁₄ aryl and up to per-halosubstituted C₃-C₁₃ heteroaryl;

wherein Q is —O—, —S—, —N(R)⁷, —(CH₂)-m, —C(O)—, —CH(OH)—,

—NR⁷C(O)NR⁷R⁷—, —NR⁷C(O)—, —C(O)NR⁷—, —(CH₂)ₘO—, —(CH₂)ₘS—,

—(CH₂)ₘN(R⁷)—, —O(CH₂)ₘ—, —CHX^{a}, —CX^{a}₂—, —S—(CH₂)ₘ— and —N(R⁷)(CH₂)ₘ—, where m=1-3, and X^{a} is halogen; and

Ar is a 5-10 member aromatic structure containing 0-4 members of the group consisting of nitrogen, oxygen and sulfur, which is unsubstituted or substituted by halogen up to per-halosubstitution and optionally substituted by Zₙ₁, wherein n1 is 0 to 3 and each Z substituent is independently selected from the group consisting of —CN, —CO₂R⁷, —C(O)NR⁷R⁷, —C(O)—NR⁷, —NO₂, —OR⁷, —SR⁷, —NR⁷R⁷, —NR⁷C(O)OR⁷, —C(O)R⁷, —NR⁷C(O)R⁷, C₁-C₁₀ alkyl, C₃-C₁₀ cycloalkyl, C₆-C₁₄ aryl, C₃-C₁₃ heteroaryl, C₇-C₂₄ alkaryl, C₄-C₂₃ alkheteroaryl, substituted C₁-C₁₀ alkyl, substituted C₃-C₁₀cycloalkyl, substituted C₇-C₂₄ alkaryl and substituted C₄-C₂₃ alkheteroaryl; wherein the one or more substituents of Z are independently selected from the group consisting of —CN, —CO₂R⁷, —C(O)NR⁷R⁷, —OR⁷, —SR⁷, —NO₂, —NR⁷R⁷, —NR⁷C(O)R⁷ and —NR⁷C(O)OR⁷;

or a pharmaceutically-acceptable salt thereof.

Exemplary compounds of these formulas include:
N-(5-tert-butyl-2-methoxyphenyl)-N′-(4-phenyloxyphenyl)urea;
N-(5-tert-butyl-2-methoxyphenyl)-N′-(4-(4-methoxyphenyloxy)phenyl)urea; N-(5-tert-butyl-2-methoxyphenyl)-N′-(4-(4-pyridinyloxy)phenyl)urea;
N-(5-tert-butyl-2-methoxyphenyl)-N′-(4-(4-pyridinylmethyl)phenyl)urea;
N-(5-tert-butyl-2-methoxyphenyl)-N′-(4-(4-pyridinylthio)phenyl)urea;
N-(5-tert-butyl-2-methoxyphenyl)-N′-(4-(4-(4,7-methano-1H-isoindole-1,3(2H)dionyl)methyl)phenyl)urea;
N-(5-tert-butyl-2-phenylphenyl)-N′-(2,3-dichlorophenyl)urea;
N-(5-tert-butyl-2-(3-thienyl)phenyl)-N′-(2,3-dichlorophenyl)urea;
N-(5-tert-butyl-2-(N-methylaminocarbonyl)methoxyphenyl)-N′-(2,3-dichlorophenyl)urea;
N-(5-tert-butyl-2-(N-methylaminocarbonyl)methoxyphenyl)-N′-(1-naphthyl)urea;
N-(5-tert-butyl-2-(N-morpholinocarbonyl)methoxyphenyl)-N′-(2,3-dichlorophenyl)urea;
N-(5-tert-butyl-2-(N-morpholinocarbonyl)methoxyphenyl)-N′-(1-naphthyl)urea;
N-(5-tert-butyl-2-(3-tetrahydrofuranyloxy)phenyl)-N′-(2,3-dichlorophenyl)urea;
N-(5-tert-butyl-2-methoxyphenyl)-N′-(4-(3-pyridinyl)methylphenyl)urea;
N-(5-trifluoromethyl-2-methoxyphenyl)-N′-(4-methylphenyl)urea;
N-(5-trifluoromethyl-2-methoxyphenyl)-N′-(4-methyl-2-fluorophenyl)urea;
N-(5-trifluoromethyl-2-methoxyphenyl)-N′-(4-fluoro-3-chlorophenyl)urea;
N-(5-trifluoromethyl-2-methoxyphenyl)-N′-(4-methyl-3-chlorophenyl)urea;
N-(5-trifluoromethyl-2-methoxyphenyl)-N′-(4-methyl-3-fluorophenyl)urea;
N-(5-trifluoromethyl-2-methoxyphenyl)-N′-(2,4-difluorophenyl)urea;
N-(5-trifluoromethyl-2-methoxyphenyl)-N′-(4-phenyloxy-3,5-dichlorophenyl)urea;
N-(5-trifluoromethyl-2-methoxyphenyl)-N′-(4-(4-pyridinylmethyl)phenyl)urea;
N-(5-trifluoromethyl-2-methoxyphenyl)-N′-(4-(4-pyridinylthio)phenyl)urea;
N-(5-trifluoromethyl-2-methoxyphenyl)-N′-(4-(4-pyridinyloxy)phenyl)urea;
N-(5-trifluoromethyl-2-methoxyphenyl)-N′-(3-(4-pyridinylthio)phenyl)urea;
N-(5-trifluoromethyl-2-methoxyphenyl)-N′-(4-(3-(N-methylaminocarbonyl)phenyloxy)phenyl)urea;
N-(5-fluorosulfonyl)-2-methoxyphenyl)-N′-(4-methylphenyl)urea;
N-(5-(difluoromethanesulfonyl)-2-methoxyphenyl)-N′-(4-methylphenyl)urea;
N-(5-(difluoromethanesulfonyl)-2-methoxyphenyl)-N′-(4-fluorophenyl)urea;
N-(5-(difluoromethanesulfonyl)-2-methoxyphenyl)-N′-(4-methyl-2-fluorophenyl)urea;
N-(5-(difluoromethanesulfonyl)-2-methoxyphenyl)-N′-(4-methyl-3-fluorophenyl)urea;
N-(5-(difluoromethanesulfonyl)-2-methoxyphenyl)-N′-(4-methyl-3-chlorophenyl)urea;
N-(5-(difluoromethanesulfonyl)-2-methoxyphenyl)-N′-(4-fluoro-3-chlorophenyl)urea;
N-(5-(difluoromethanesulfonyl)-2-methoxyphenyl)-N′-(4-fluoro-3-methylphenyl)urea;
N-(5-(difluoromethanesulfonyl)-2-methoxyphenyl)-N′-(2,3-dimethylphenyl)urea;
N-(5-(trifluoromethanesulfonyl)-2-methoxphenyl)-N′-(4-methylphenyl)urea;
N-(3-methoxy-2-naphthyl)-N′-(2-fluorophenyl)urea;
N-(3-methoxy-2-naphthyl)-N′-(4-methylphenyl)urea;
N-(3-methoxy-2-naphthyl)-N′-(3-fluorophenyl)urea;
N-(3-methoxy-2-naphthyl)-N′-(4-methyl-3-fluorophenyl)urea;
N-(3-methoxy-2-naphthyl)-N′-(2,3-dimethylphenyl)urea;
N-(3-methoxy-2-naphthyl)-N′-(1-naphthyl)urea;
N-(3-methoxy-2-naphthyl)-N′-(4-(4-pyridinylmethyl)phenyl)urea;
N-(3-methoxy-2-naphthyl)-N′-(4-(4-pyridinylthio)phenyl)urea;
N-(3-methoxy-2-naphthyl)-N′-(4-(4-methoxyphenyloxy)phenyl)urea;
N-(3-methoxy-2-naphthyl)-N′-(4-(4-(4,7-methano-1H-isoindole-1,3(2H)dionyl)methyl)phenyl)urea;
N-(2-hydroxy-4-nitro-5-chlorophenyl)-N′-(phenyl)urea;
N-(2-hydroxy-4-nitro-5-chlorophenyl)-N′-(4-(4-pyridinylmethyl)phenyl)urea;
   and pharmaceutically acceptable salts thereof.

Such compounds are described in published PCT applications WO 96/21452, WO 96/40143, WO 97/25046, WO 97/35856, WO 98/25619, WO 98/56377, WO 98/57966, WO 99/32110, WO 99/32121, WO 99/32463, WO 99/61440, WO 99/64400, WO 00/10563, WO 00/17204, WO 00/19824, WO 00/41698, WO 00/64422, WO 00/71535, WO 01/38324, WO 01/64679, WO 01/66539, and WO 01/66540, each of which is herein incorporated by reference in their entirety.

In all instances herein where there is an alkenyl or alkynyl moiety as a substituent group, the unsaturated linkage, i.e., the vinylene or acetylene linkage, is preferably not directly attached to the nitrogen, oxygen or sulfur moieties, for instance in OR_{f}, or for certain R₂ moieties.

As used herein, “optionally substituted” unless specifically defined shall mean such groups as halogen, such as fluorine, chlorine, bromine or iodine; hydroxy; hydroxy-substituted C₁₋₁₀alkyl; C₁₋₁₀ alkoxy, such as methoxy or ethoxy; S(O)ₘ alkyl, wherein m is 0, 1 or 2, such as methyl thio, methylsulfinyl or methyl sulfonyl; amino, mono and di-substituted amino, such as in the NR₇R₁₇ group; or where the R₇R₁₇ can together with the nitrogen to which they are attached cyclize to form a 5- to 7-membered ring which optionally includes an additional heteroatom selected from O, N, and S; C₁₋₁₀ alkyl, cycloalkyl, or cycloalkyl alkyl group, such as methyl, ethyl, propyl, isopropyl, t-butyl, etc. or cyclopropyl methyl; halo-substituted C₁₋₁₀ alkyl, such as CF₃; an optionally substituted aryl, such as phenyl, or an optionally substituted arylalkyl, such as benzyl or phenethyl, wherein these aryl moieties can also be substituted one to two times by halogen; hydroxy; hydroxy-substituted alkyl; C₁₋₁₀ alkoxy; S(O)ₘ alkyl; amino, mono- and di-substituted amino, such as in the NR₇R₁₇ group; alkyl, or CF₃.

Inhibitors useful in the present invention can be used with any pharmaceutically acceptable salt. The term “pharmaceutically acceptable salts” refers to salts prepared from pharmaceutically acceptable non-toxic bases or acids. When the compound utilized by the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Basic salts of inorganic and organic acids also include as hydrochloric acid, hydrobromic acid, sulphuric acid, phosphoric acid, methane sulphonic acid, ethane sulphonic acid, acetic acid, malic acid, tartaric acid, citric acid, lactic acid, oxalic acid, succinic acid, fumaric acid, maleic acid, benzoic acid, salicylic acid, phenylacetic acid and mandelic acid. In addition, pharmaceutically-acceptable salts of the above-described compounds can also be formed with a pharmaceutically-acceptable cation, for instance, if a substituent group comprises a carboxy moiety. Suitable pharmaceutically-acceptable cations are well known to those skilled in the art and include alkaline, alkaline earth, ammonium and quaternary ammonium cations.

Other pharmaceutically acceptable organic non-toxic bases from which salts can be formed include ion exchange resins such as, for example, arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. Synthesis of the disclosed compounds is discussed in U.S. patent application Ser. No. 09/575,060, which is hereby incorporated by reference in its entirety.

The inhibitors of p38 MAP kinase can be used as single therapeutic agents or in combination with other therapeutic agents. Drugs that could be usefully combined with these compounds include monoclonal antibodies targeting cells of the immune system, antibodies or soluble receptors or receptor fusion proteins targeting immune or non-immune cytokines, and small molecule inhibitors of cell division, protein synthesis, or mRNA transcription or translation, or inhibitors of immune cell differentiation, activation, or function (e.g., cytokine secretion). In addition, p38 inhibitors may be used in combination with other pain relieving compounds to promote efficacy or alleviate detrimental side effects associated therewith. For instance, p38 can alleviate detrimental side effects associated with opiates and other pain medications, such effects including but not limited to immunosuppression, tachyphylaxis, and systemic infection. See for example Singhal et al, Journal of Immunology, April 15; 168(8), 4025-33 (2002). Coadministration of p38 inhibitors with opiates would allow for a reduced amount of opiates to be used, thus minimizing negative side effects while maintaining the beneficial results of opiate-mediated analgesia. Thus, the coadministration of these compounds can be considered to yield a synergistic effect.

The following terms, as used herein, refer to:

“halo” or “halogens”, include the halogens: chloro, fluoro, bromo and iodo;

“C₁₋₁₀alkyl” or “alkyl”—both straight and branched chain radicals of 1 to 10 carbon atoms, unless the chain length is otherwise limited, including, but not limited to, methyl, ethyl, n-propyl, iso-propyl, n-butyl, sec-butyl, iso-butyl, tert-butyl, n-pentyl and the like;

the term “cycloalkyl” is used herein to mean cyclic radicals, preferably of 3 to 8 carbons, including but not limited to cyclopropyl, cyclopentyl, cyclohexyl, and the like;

the term “cycloalkenyl” is used herein to mean cyclic radicals, preferably of 5 to 8 carbons, which have at least one double bond, including but not limited to cyclopentenyl, cyclohexenyl, and the like;

the term “alkenyl” is used herein at all occurrences to mean straight or branched chain radical of 2-10 carbon atoms, unless the chain length is limited thereto, wherein there is at least one double bond between two carbon atoms in the chain, including, but not limited to ethenyl, 1-propenyl, 2-propenyl, 2-methyl-1-propenyl, 1-butenyl, 2-butenyl and the like;

“aryl”—phenyl and naphthyl;

“heteroaryl” (on its own or in any combination, such as “heteroaryloxy” or “heteroaryl alkyl”)—a 5-10-membered aromatic ring system in which one or more rings contain one or more heteroatoms selected from the group consisting of N, O and S, such as, but not limited, to pyrrole, pyrazole, furan, thiophene, quinoline, isoquinoline, quinazolinyl, pyridine, pyrimidine, oxazole, thiazole, thiadiazole, triazole, imidazole, or benzimidazole;

“heterocyclic” (on its own or in any combination, such as “heterocyclylalkyl”)—a saturated or partially unsaturated 4-10-membered ring system in which one or more rings contain one or more heteroatoms selected from the group consisting of N, O, and S; such as, but not limited to, pyrrolidine, piperidine, piperazine, morpholine, tetrahydropyran, or imidazolidine;

the term “aralkyl” or “heteroarylalkyl” or “heterocyclicalkyl” is used herein to mean C₁₋₄ alkyl as defined above attached to an aryl, heteroaryl or heterocyclic moiety as also defined herein unless otherwise indicate;

“sulfinyl”—the oxide S(O) of the corresponding sulfide, the term “thio” refers to the sulfide, and the term “sulfonyl” refers to the fully oxidized S(O)₂ moiety;

“aroyl”—a C(O)Ar, wherein Ar is as phenyl, naphthyl, or aryl alkyl derivative such as defined above, such groups include but are not limited to benzyl and phenethyl; and

“alkanoyl”—a C(O)C₁₋₁₀ alkyl wherein the alkyl is as defined above.

For the purposes herein the “core” 4-pyrimidinyl moiety for R₁ or R₂ is referred to as the formula:

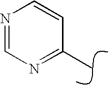

The compounds useful in the practice of the present invention can contain one or more asymmetric carbon atoms and can exist in racemic and optically active forms. The use of all of these compounds are included within the scope of the present invention.

Compounds useful in the practice of the present invention also'include, but are not limited to, the compounds shown in Tables 1 and 2, below.

TABLE 1

| | Citations, each of which is herein |
|---|---|
| Chemical Structure | incorporated by reference. |
| 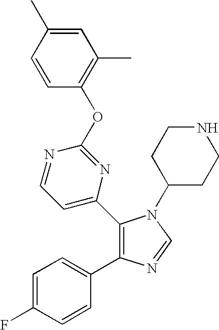 | WO-00166539, WO-00166540, WO-00164679, WO-00138324, WO-00064422, WO-00019824, WO-00010563, WO-09961440, WO-09932121, WO-09857966, WO-09856377, WO-09825619, WO-05756499, WO-09735856, WO-09725046, WO-09640143, WO-09621452; Gallagher, T. F., et. Al., Bioorg. Med Chem. 5: 49 |
| # (1997); Adams, J. L., et al., Bioorg. Med. Chem. Lett. 8: 3111- 3116 (1998) | |
| 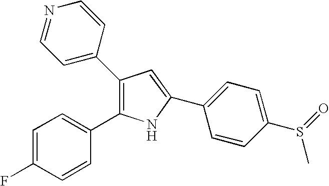 | De Laszlo, S. E., et. Al., Bioorg Med Chem Lett. 8: 2698 (1998) |
| 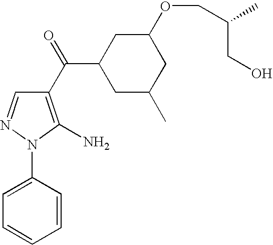 | WO-09957101; Poster presentation at the 5^{th} World Congress on Inflammation, Edinburgh, UK. (2001) |
| 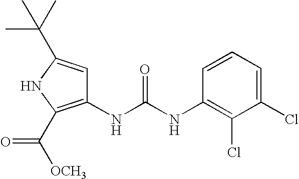 | WO-00041698, WO-09932110, WO-09932463 |
| 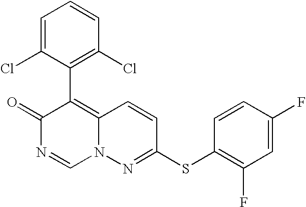 | WO-00017204, WO-09964400 |
| 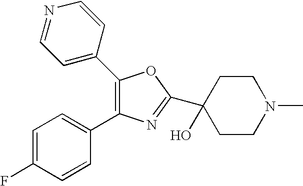 | Revesz. L., et al., Bioorg Med Chem Lett. 10: 1261 (2000) |
| 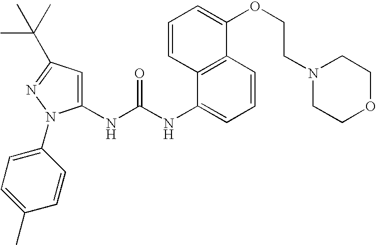 | WO-00207772 |
| 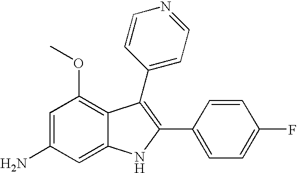 | Fijen, J. W., et al., Clin. Exp. Immunol. 124: 16-20 (2001); Wadsworth, S. A., el. at., J. Pharmacol. Expt. Therapeut. 291: 680 (1999) |
| 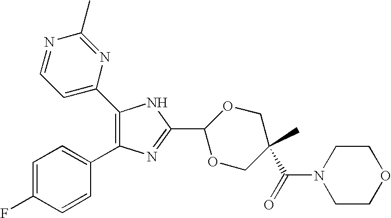 | Collis, A. J., et at.. Bioorg. Med Chem. Lett. 11: 693-696 (2001); McLay, L. M., et al., Bioorg Med Chem 9: 537-554 (2001) |
| 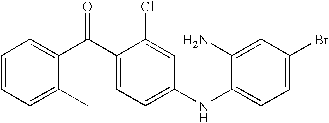 | WO-00110865, WO-00105749 |

TABLE 2
Exemplary p38 Inhibitors

| Cpd. | |
|---|---|
| # | Mol. Structure |
| 1 | 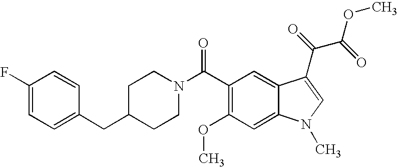 |
| 2 | 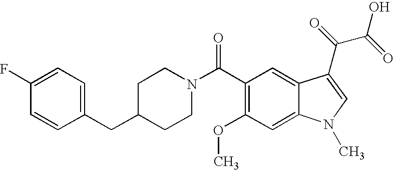 |
| 3 | 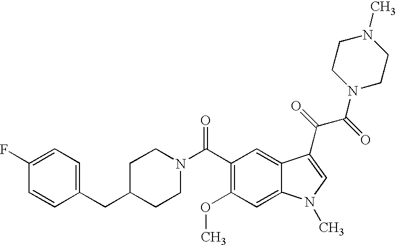 |
| 4 | 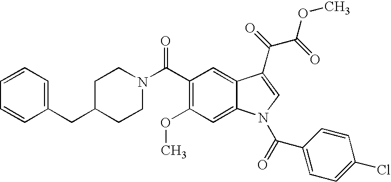 |
| 5 | 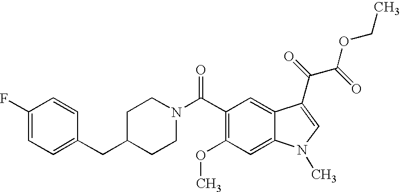 |
| 6 | 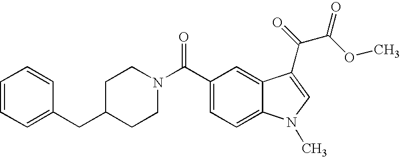 |
| 7 | 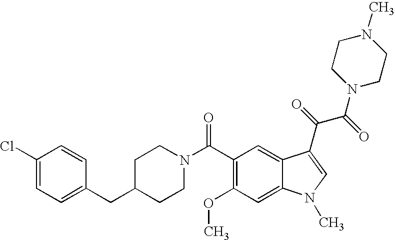 |
| 8 | 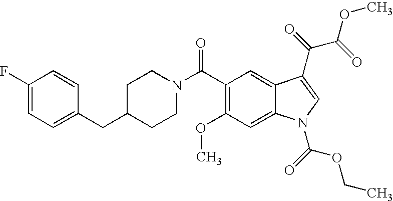 |
| 9 | 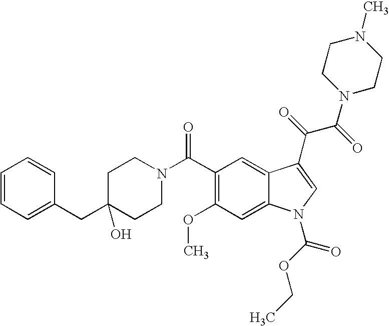 |
| 10 | 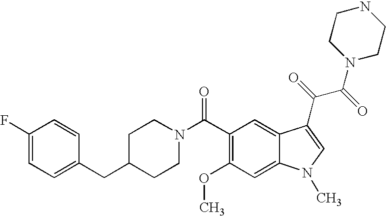 |
| 11 | 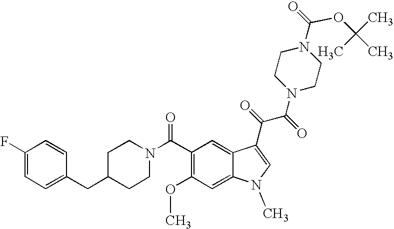 |
| 12 | 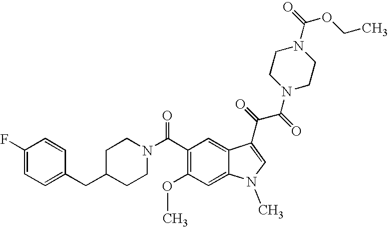 |
| 13 | 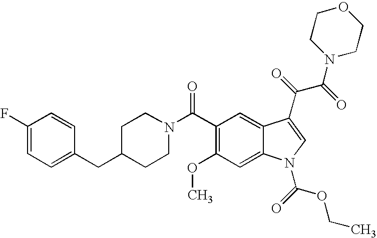 |
| 14 | 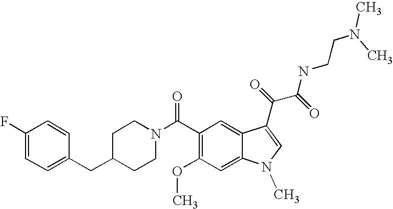 |
| 15 | 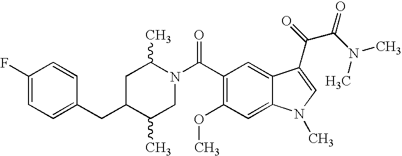 |
| 16 | 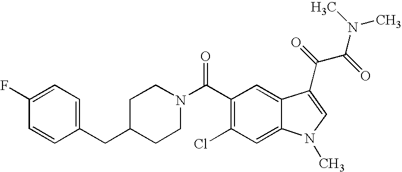 |
| 17 | 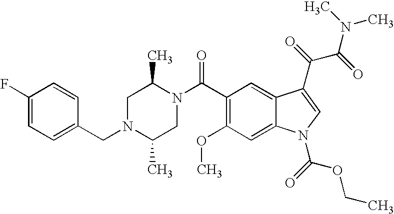 |
| 18 | 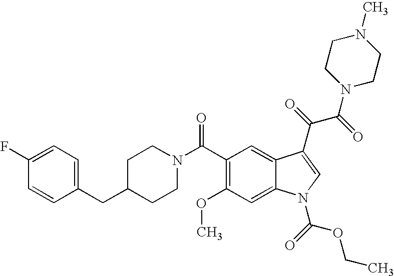 |
| 19 | 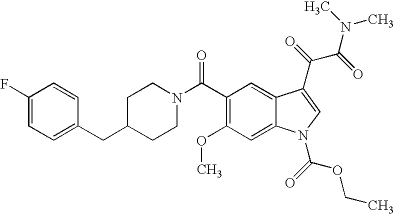 |
| 20 | 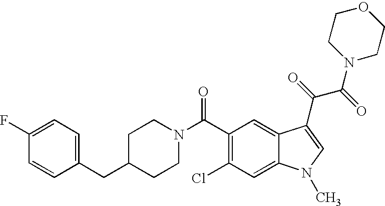 |
| 21 | 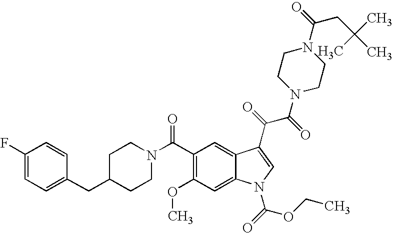 |
| 22 | 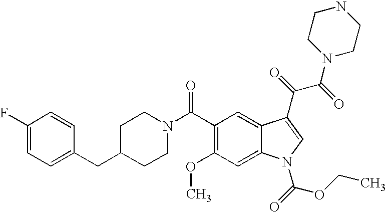 |
| 23 | 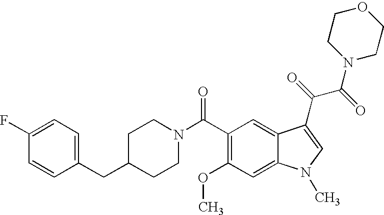 |
| 24 | 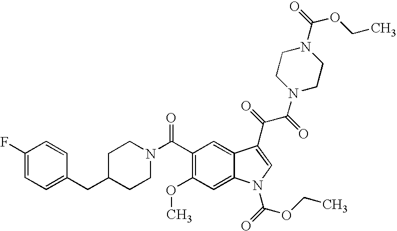 |
| 25 | 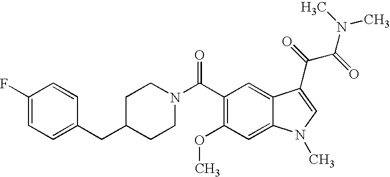 |
| 26 | 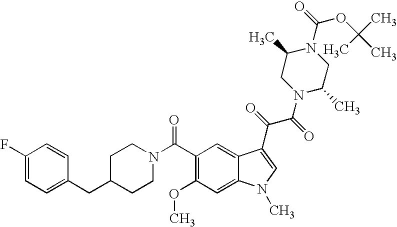 |
| 27 | 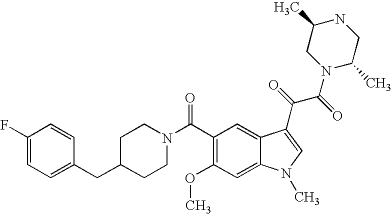 |
| 28 | 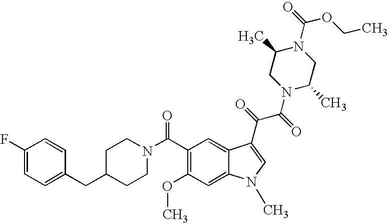 |
| 29 | 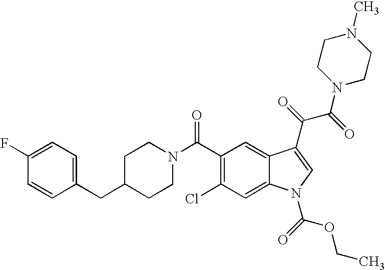 |
| 30 | 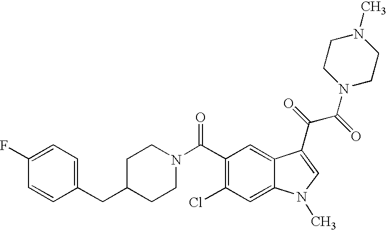 |
| 31 | 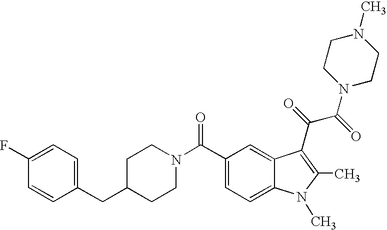 |
| 32 | 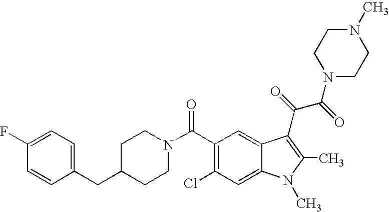 |
| 33 | 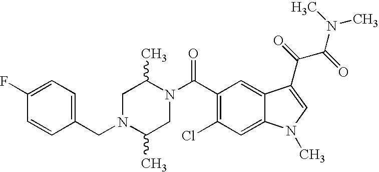 |
| 34 | 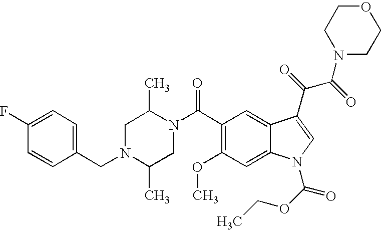 |
| 35 | 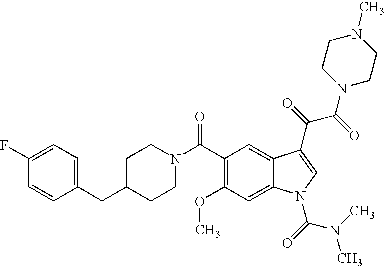 |
| 36 | 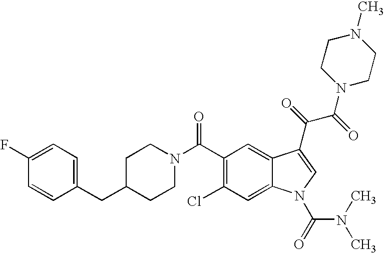 |
| 37 | 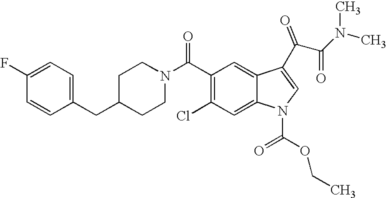 |
| 38 | 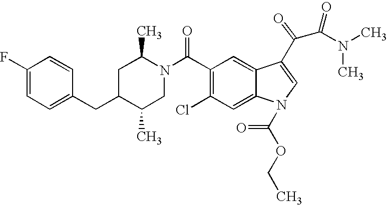 |
| 39 | 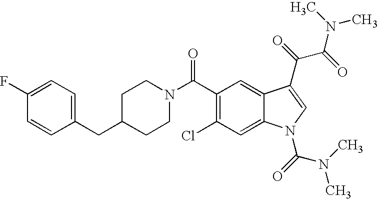 |
| 40 | 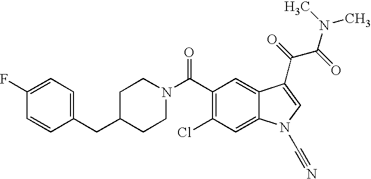 |
| 41 | 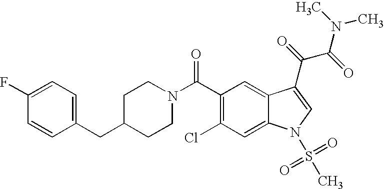 |
| 42 | 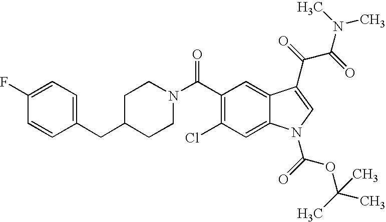 |
| 43 | 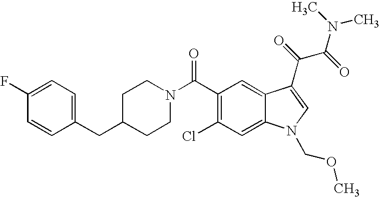 |
| 44 | 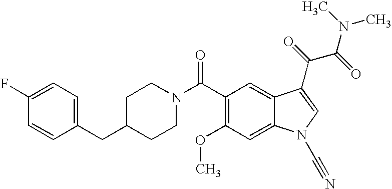 |
| 45 | 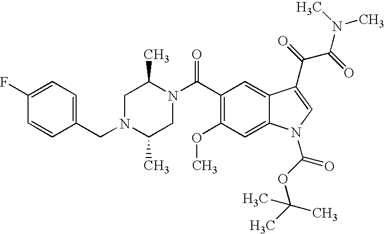 |
| 46 | 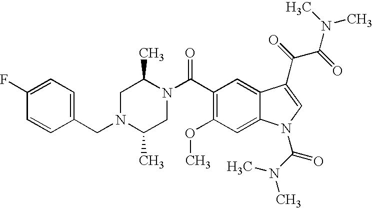 |
| 47 | 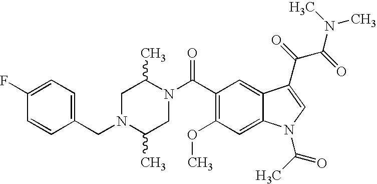 |
| 48 | 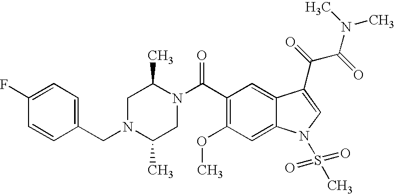 |
| 49 | 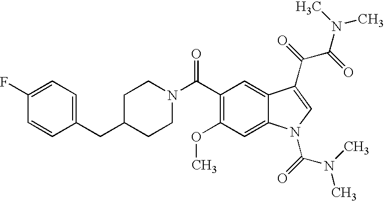 |
| 50 | 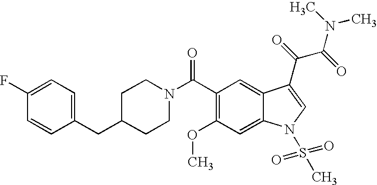 |
| 51 | 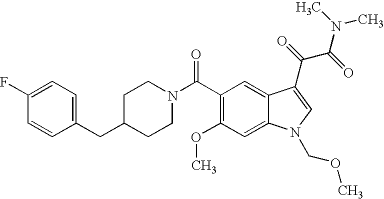 |
| 52 | 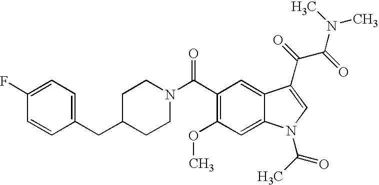 |
| 53 | 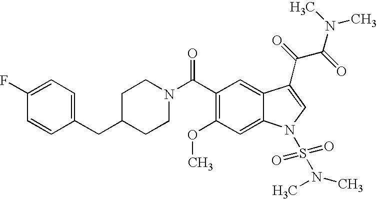 |
| 54 | 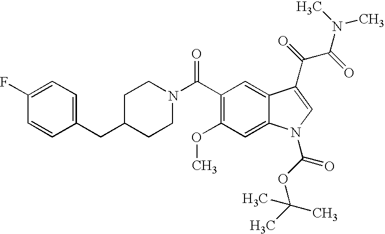 |
| 55 | 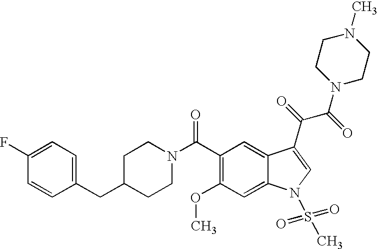 |
| 56 | 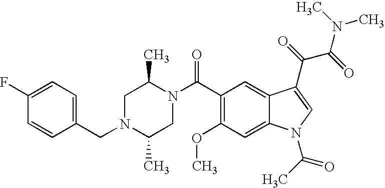 |
| 57 | 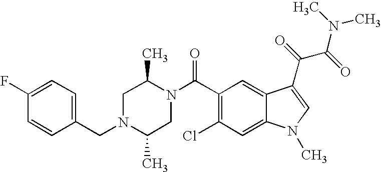 |
| 58 | 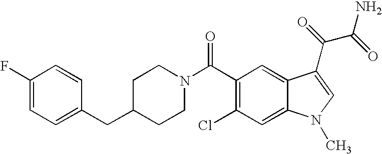 |
| 59 | 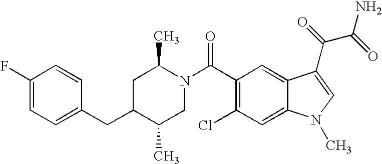 |
| 60 | 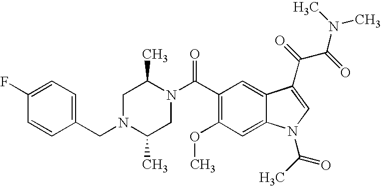 |
| 61 | 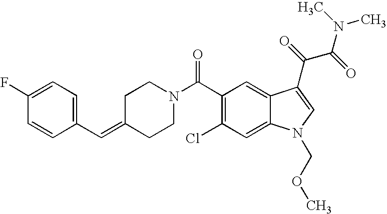 |
| 62 | 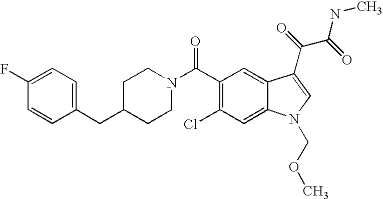 |
| 63 | 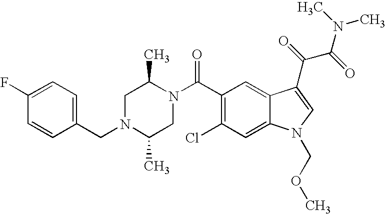 |
| 64 | 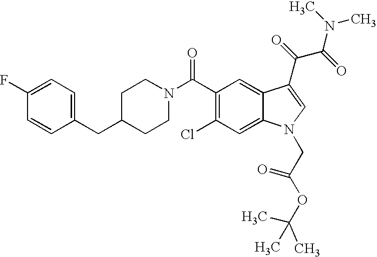 |
| 65 | 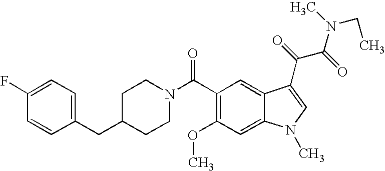 |
| 66 | 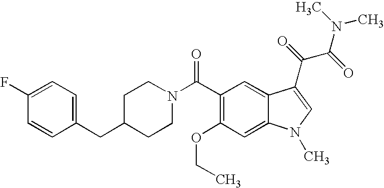 |
| 67 | 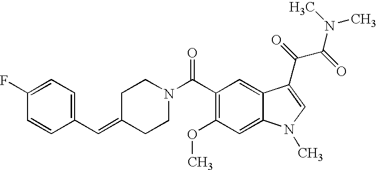 |
| 68 | 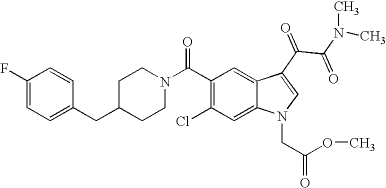 |
| 69 | 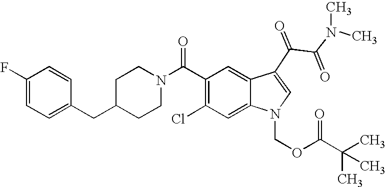 |
| 70 | 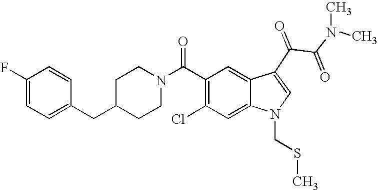 |
| 71 | 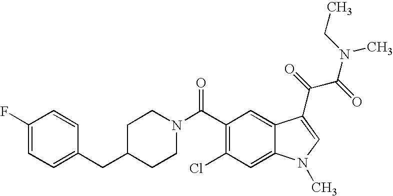 |
| 72 | 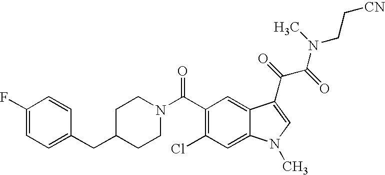 |
| 73 | 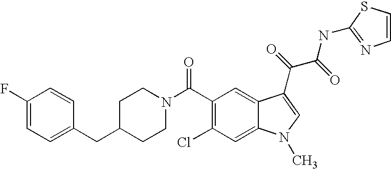 |
| 74 | 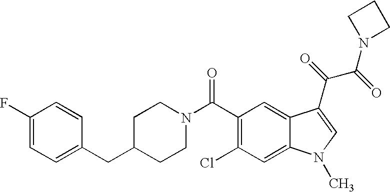 |
| 75 | 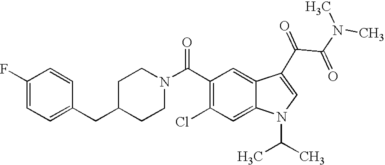 |
| 76 | 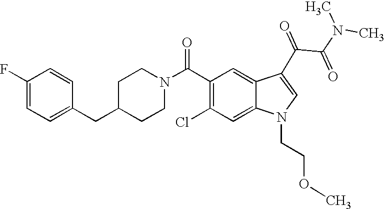 |
| 77 | 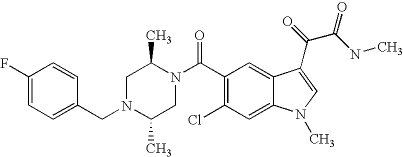 |
| 78 | 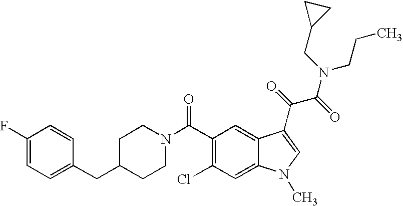 |
| 79 | 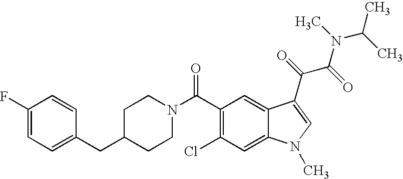 |
| 80 | 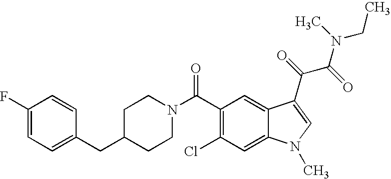 |
| 81 | 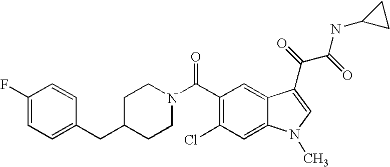 |
| 82 | 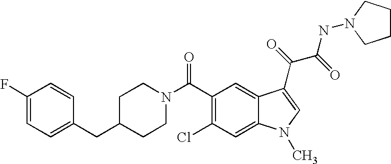 |
| 83 | 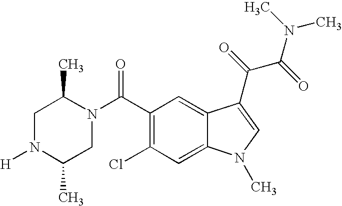 |
| 84 | 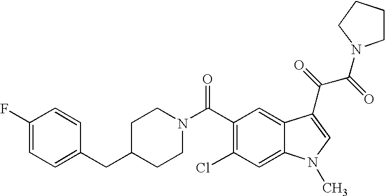 |
| 85 | 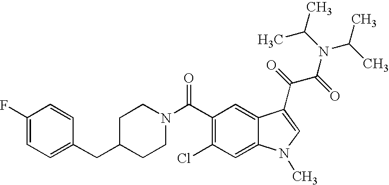 |
| 86 | 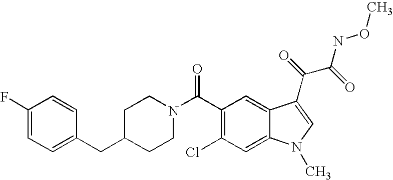 |
| 87 | 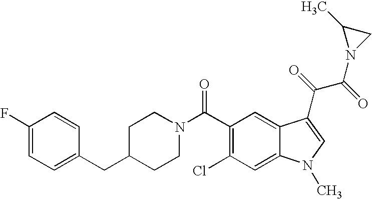 |
| 88 | 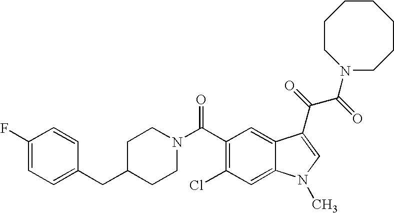 |
| 89 | 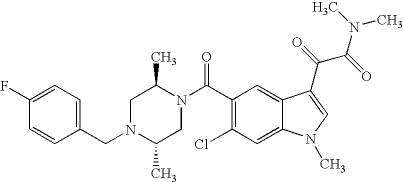 |
| 90 | 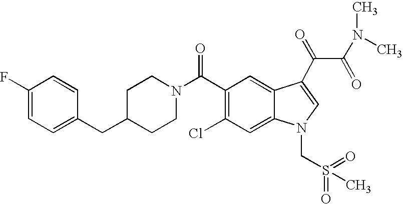 |
| 91 | 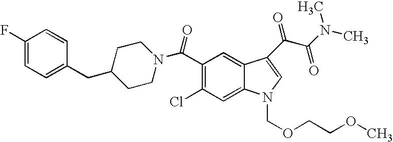 |
| 92 | 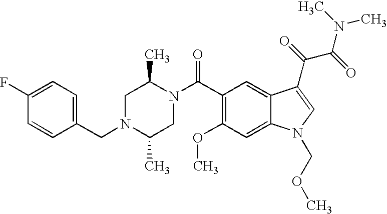 |
| 93 | 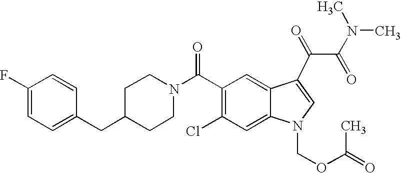 |
| 94 | 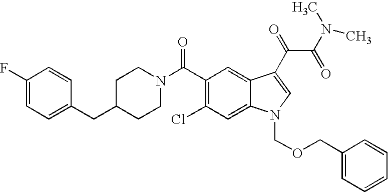 |
| 95 | 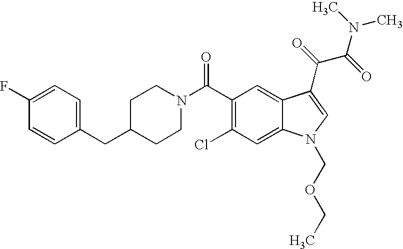 |
| 96 | 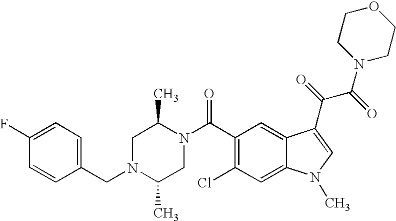 |
| 97 | 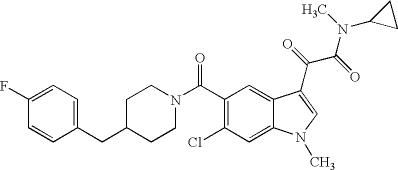 |
| 98 | 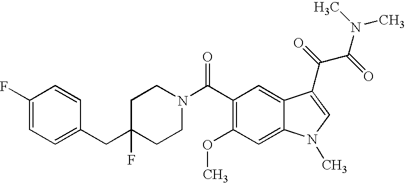 |
| 99 | 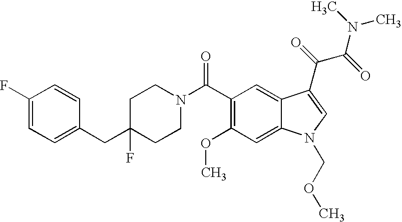 |
| 100 | 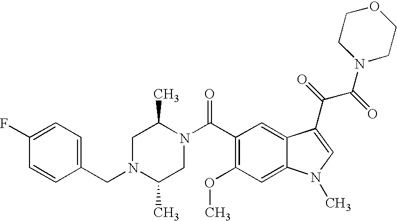 |
| 101 | 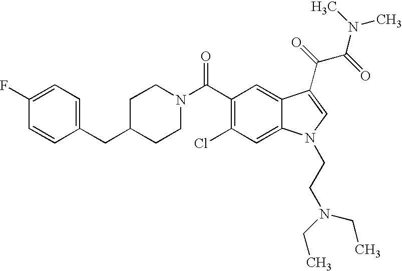 |
| 102 | 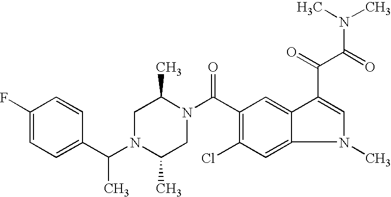 |
| 103 | 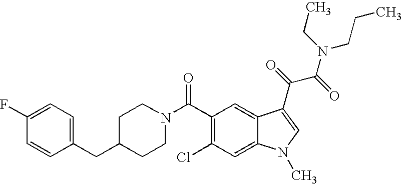 |
| 104 | 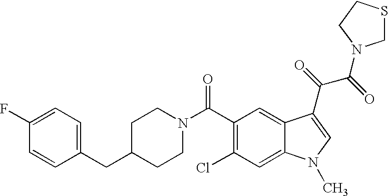 |
| 105 | 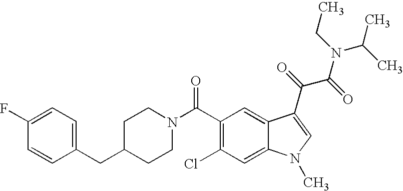 |
| 106 | 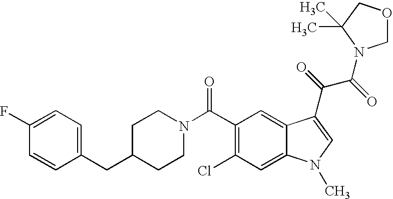 |
| 107 | 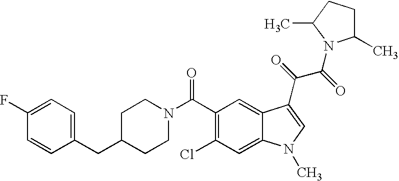 |
| 108 | 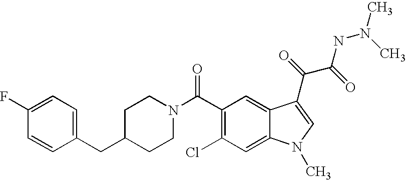 |
| 109 | 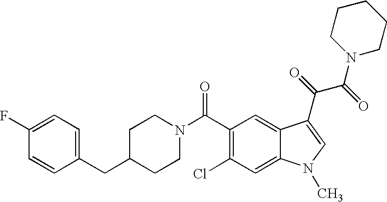 |
| 110 | 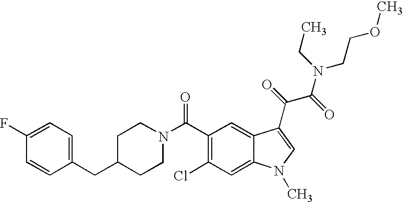 |
| 111 | 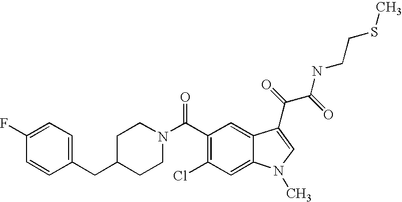 |
| 112 | 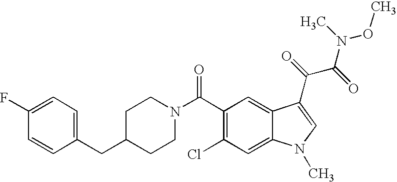 |
| 113 | 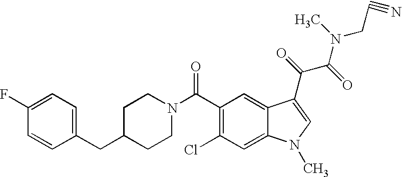 |
| 114 | 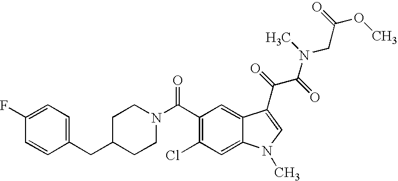 |
| 115 | 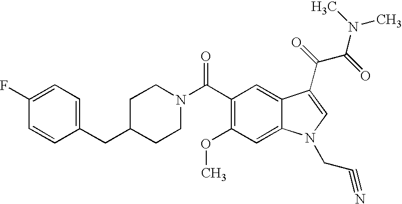 |
| 116 | 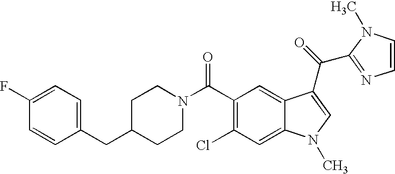 |
| 117 | 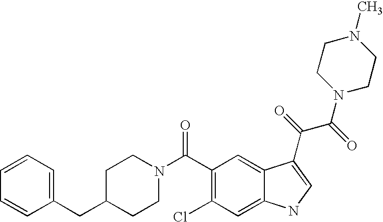 |
| 118 | 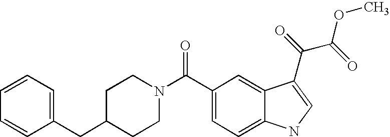 |
| 119 | 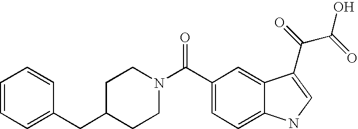 |
| 120 | 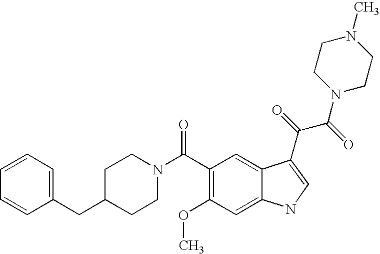 |
| 121 | 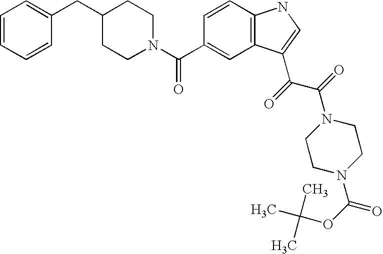 |
| 122 | 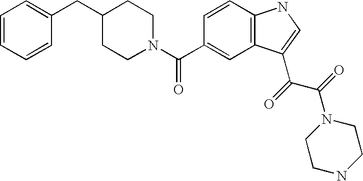 |
| 123 | 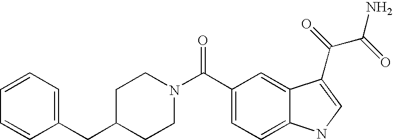 |
| 124 | 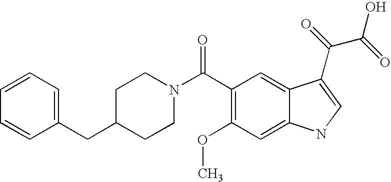 |
| 125 | 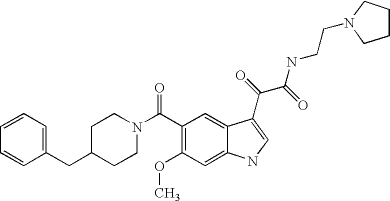 |
| 126 | 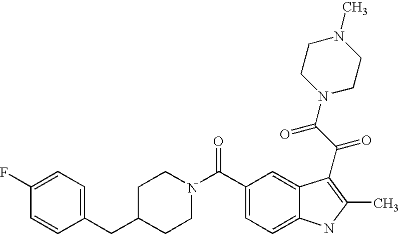 |
| 127 | 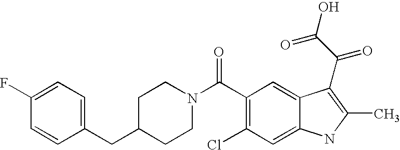 |
| 128 | 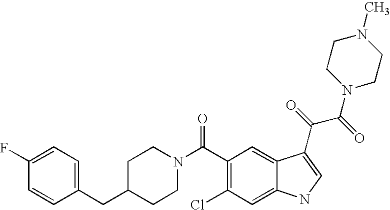 |
| 129 | 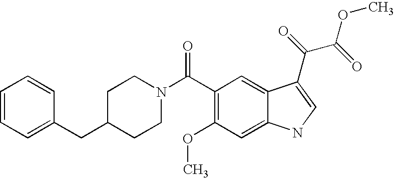 |
| 130 | 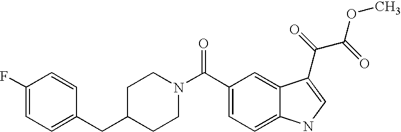 |
| 131 | 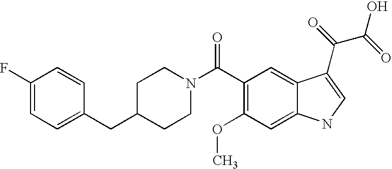 |
| 132 | 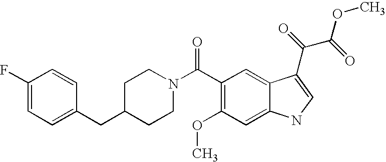 |
| 133 | 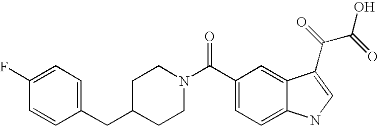 |
| 134 | 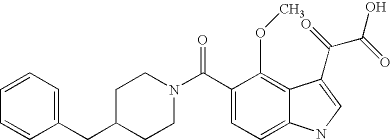 |
| 135 | 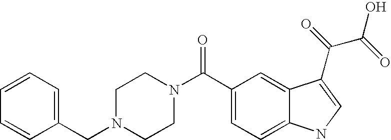 |
| 136 | 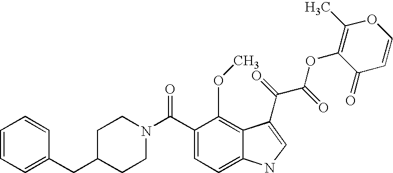 |
| 137 | 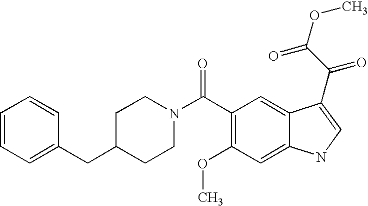 |
| 138 | 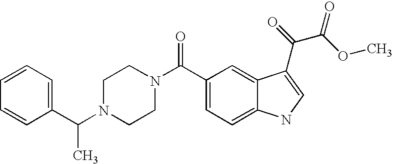 |
| 139 | 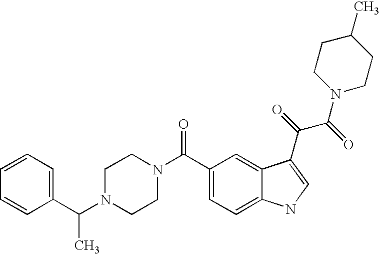 |
| 140 | 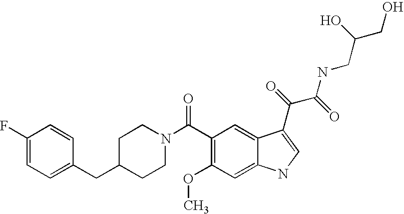 |
| 141 | 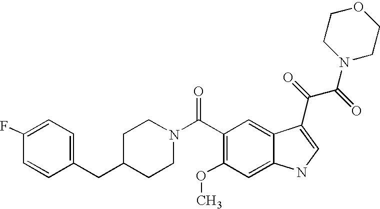 |
| 142 | 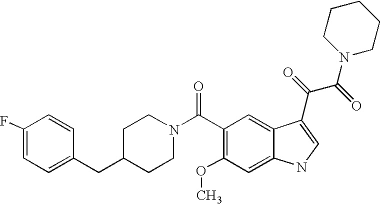 |
| 143 | 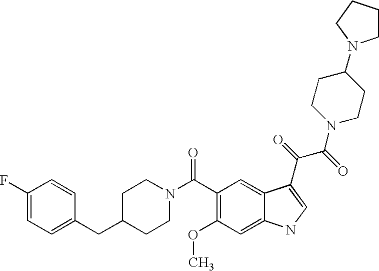 |
| 144 | 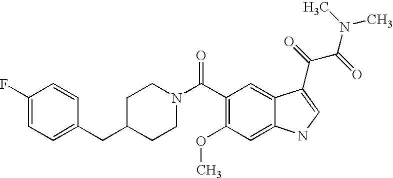 |
| 145 | 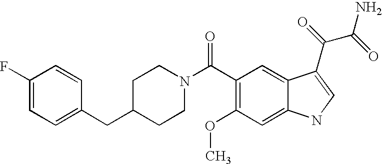 |
| 146 | 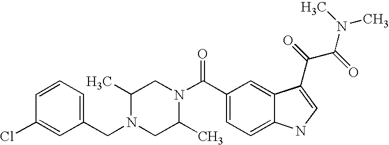 |
| 147 | 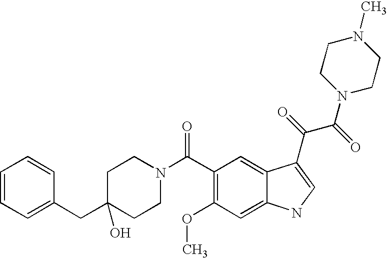 |
| 148 | 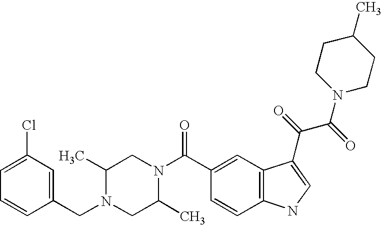 |
| 149 | 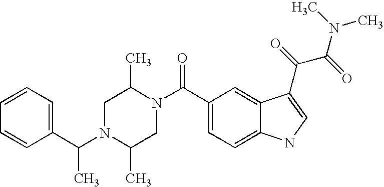 |
| 150 | 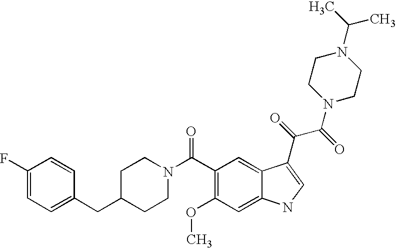 |
| 151 | 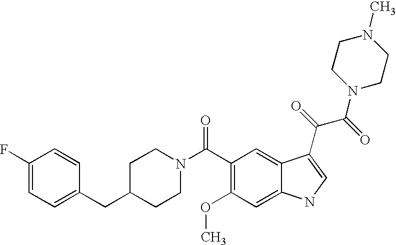 |
| 152 | 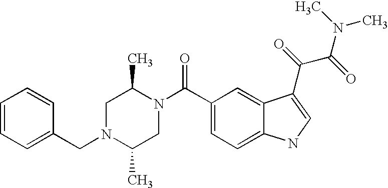 |
| 153 | 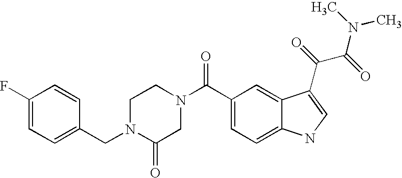 |
| 154 | 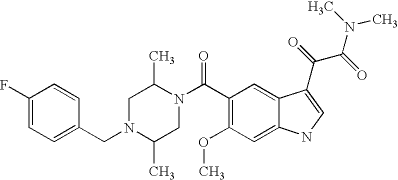 |
| 155 | 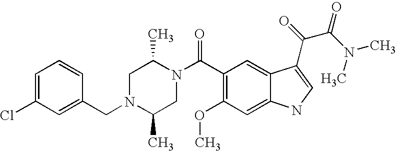 |
| 156 | 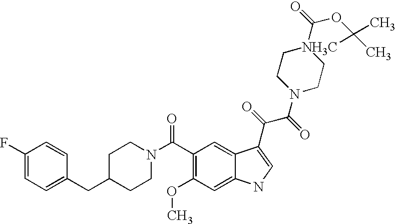 |
| 157 | 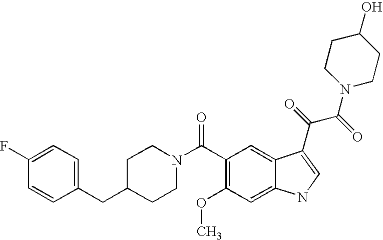 |
| 158 | 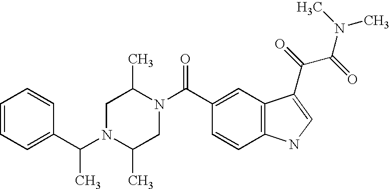 |
| 159 | 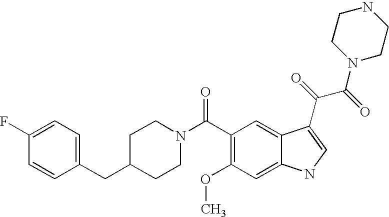 |
| 160 | 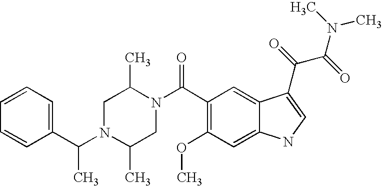 |
| 161 | 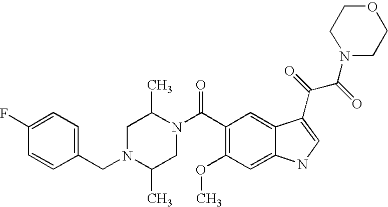 |
| 162 | 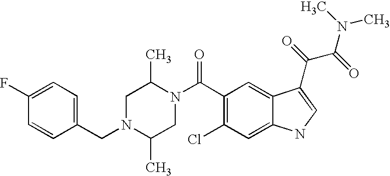 |
| 163 | 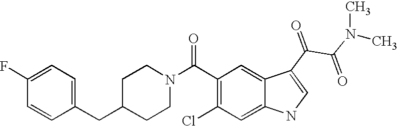 |
| 164 | 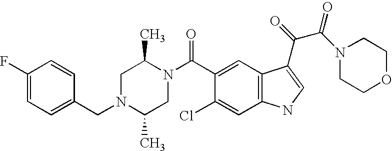 |
| 165 | 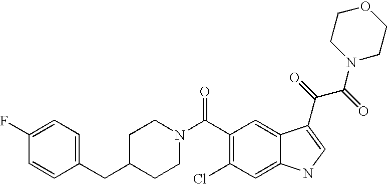 |
| 166 | 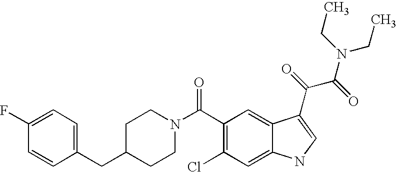 |
| 167 | 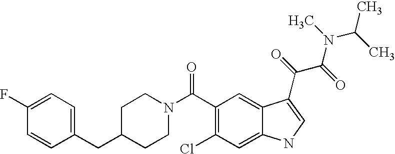 |
| 168 | 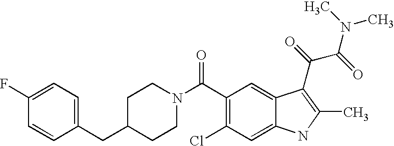 |
| 169 | 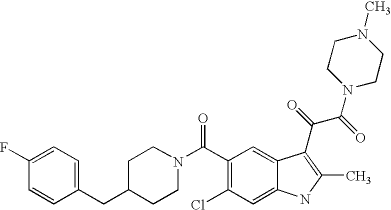 |
| 170 | 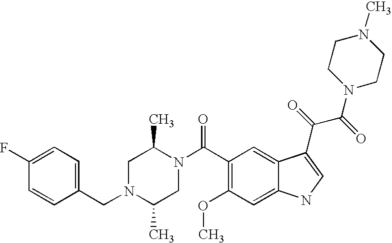 |
| 171 | 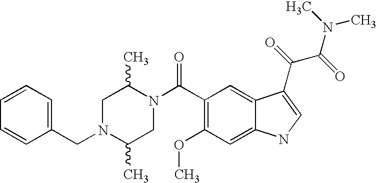 |
| 172 | 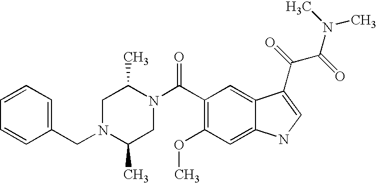 |
| 173 | 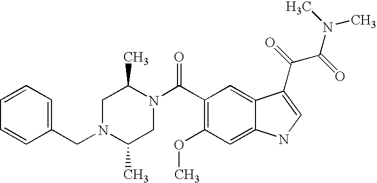 |
| 174 | 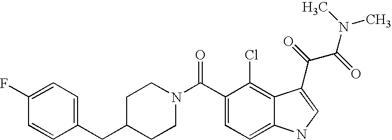 |
| 175 | 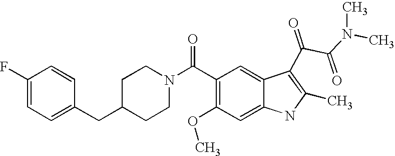 |
| 176 | 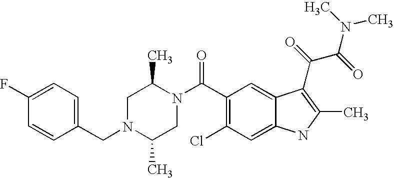 |
| 177 | 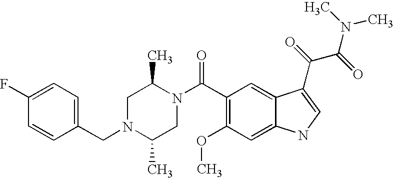 |
| 178 | 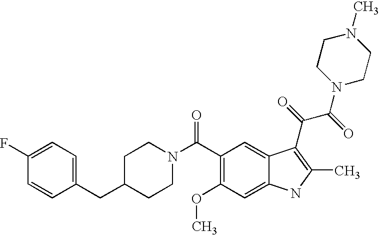 |
| 179 | 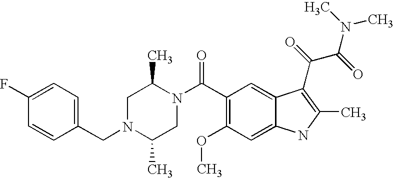 |
| 180 | 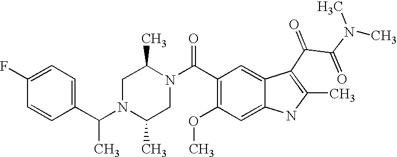 |
| 181 | 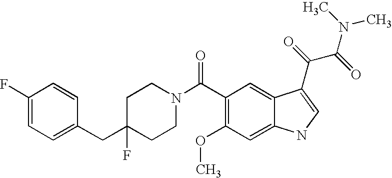 |

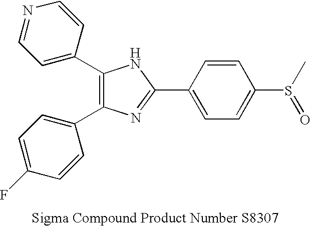

The compounds described above are provided for guidance and exemplary purposes only. It should be understood that any modulator of p38 MAP kinase is useful for the invention provided that it exhibits adequate activity relative to the targeted protein.

The following examples are offered to illustrate but not to limit the invention.

### EXAMPLES

In the following examples, a variety of different small molecule inhibitors were evaluated for their effect on the prevention or treatment of elicited pain responses. As provided herein SA (compound 15, Table B), SB (pyridinyl imidazole based compound that is known in the literature as a p38 MAPK modulator and is commercial available through Sigma-Aldrich® under product number S8307), SC (compound 57, Table B), SD (compound 183, Table B), SE (compound 154, Table B), SF (compound 2, Table B), SG (compound 3, Table B), SH (compound 84, Table B), SI (compound 92, Table B), SJ (compound 96, Table B), SK (compound 141, Table B), SL (compound 169, Table B), SM (compound 67, Table B) are compounds that generally exhibit p38 MAPK activity with a relative IC₅₀ value of less than 5 nM, as observed in an assay similar to the phosphorylation assay disclosed above (see Kumar). Various methods of administration were utilized including oral, intravenous, and intrathecal routes.

### Example 1

### Presence and Activity of p38 MAP Kinase in the Central Nervous System

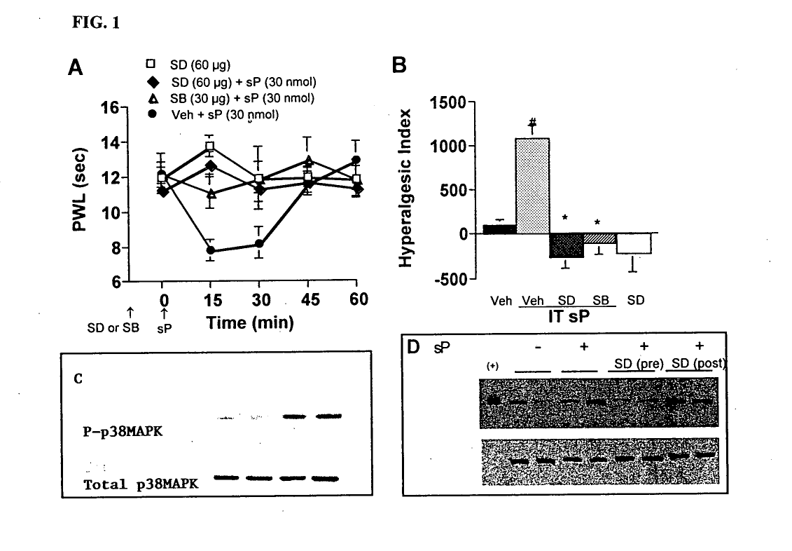

As shown in FIG. 1C, spinal cord protein extracts were examined by Western blotting and revealed that p38 MAPK is constitutively expressed under resting conditions. Spinal cords from rats were obtained after decapitation and hydroextrusion. Lumbar dorsal horns were processed for Western blot analysis using rabbit anti-P-p38 and rabbit anti-p38 antiserum (1:1000, Cell Signaling Technology) and COX-1 or COX-2 antibodies (1:500, Cayman). Immunopositive bands were detected by ECL. As noted, p38 MAPK is activated in its phosphorylated state (P-p38 MAPK), a form which was found to be constitutively present in low levels in dorsal horn tissue obtained from spinal cord after intrathecal (IT) injection of saline (FIG. 1C). However, IT administration of sP, in a dose that results in a potent NK1-receptor mediated thermal hyperalgesia (FIG. 1A,B), produced substantial increases in dorsal horn P-p38 MAPK (FIG. 1C).

The Effect of p38 MAPK Inhibitors on Hyperalgesia Induced by IT sP or NMDA

To determine the role of spinal p38 MAPK activation in pain behavior, an examination was conducted to determine the efficacy of SB203580 (SB), a moderately active p38 MAPK inhibitor and SD, an inhibitor with a preferential activity to p38 MAPK-α, against thermal hyperalgesia evoked by intrathecal injection of substance P (sP). (The inhibitory activity of SD for p38 alpha and p38 beta was evaluated with an assay using recombinant (E. coli) human enzymes and myelin basic protein as a substrate by the methods of Clerk and Kumar respectively. See A. Clerk, P. HOURS. Sugden, FEBS Lett 426, 93-6. (1998) and S. Kumar, et al., Biochem Biophys Res Commun 235, 533-8. (1997).

Male Sprague-Dawley rats (300-350 g) were implanted under isoflurane anesthesia with lumbar polyethylene (PE-10) catheters according to a modified method originally described in Physiol. Behav. 17, 1031 (1976). IT injection studies were carried out 5-8 days after surgery and all agents were injected in 10 μl followed by 10 μl to flush catheter. sP and SB203580 were dissolved in physiological saline, and SD was dissolved 5% DMSO (which was used as the control vehicle). IT saline and 5% DMSO alone had no effect on behavior or protein expression/phosphorylation.

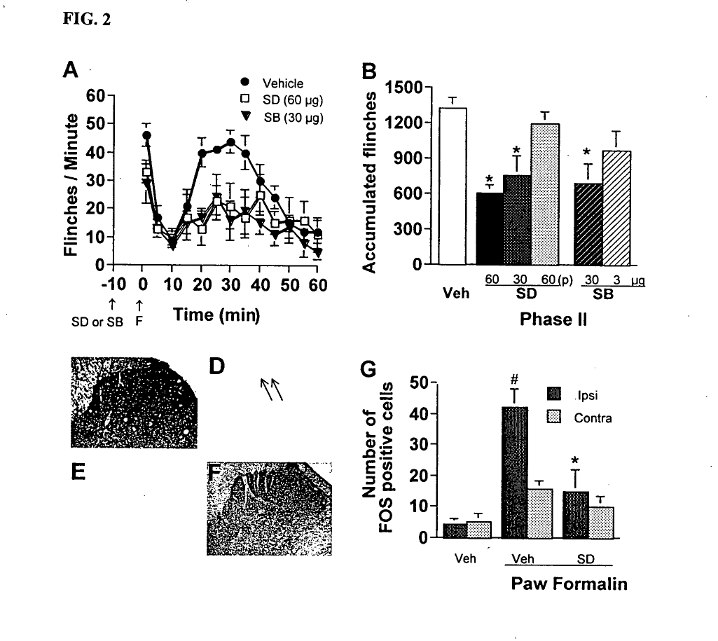

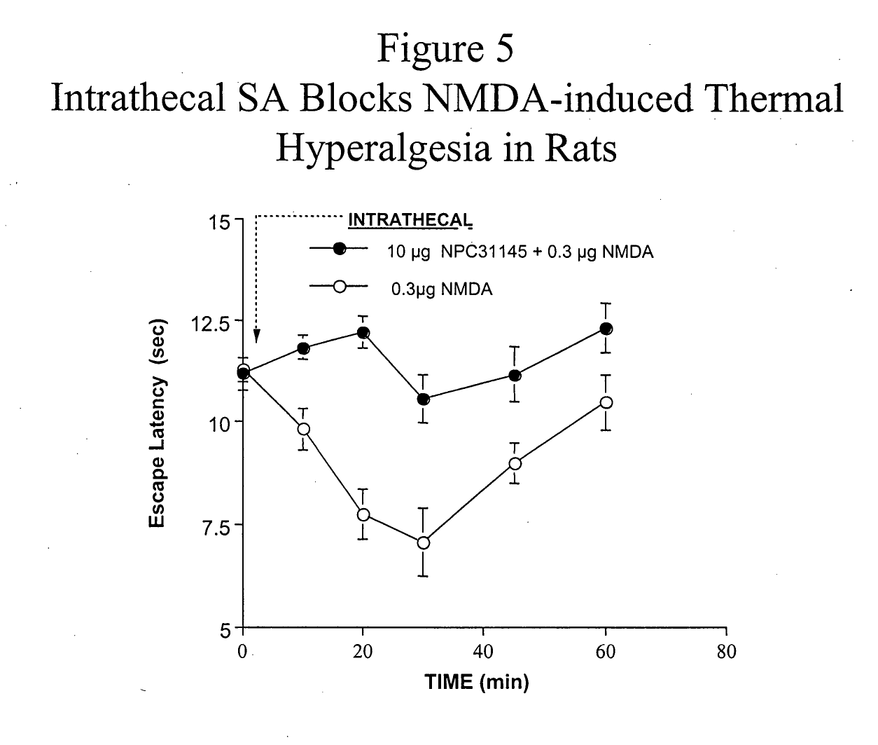

As shown in FIG. 2, both IT SD and SB blocked the thermal hyperalgesia induced by IT sP in a dose dependent fashion. The intrathecal delivery of NMDA (0.3 μg) produces a comparable hyperalgesia that is reversed by NMDA antagonists (MK801: 10 μg) and this hyperalgesia is also reversed in a dose dependent fashion by IT SD (3-60 μg) and IT SB (1-30 μg). Another p38 MAPK inhibitor, SA, exhibited similar attenuation of NMDA-induced hyperalgesia as shown in FIG. 5.

p38 MAPK Activation Relative to Altered Spinal Activity (Formalin Mediated Hyperalgesia)

An evaluation was conducted to determine presence or lack thereof for an afferent-mediated induction of phosphorylated (activated) spinal p38 MAPK. A formalin mediated hyperalgesia model was utilized to conduct the evaluation. (In the formalin model, a standard dose of formalin is injected into the rat paw, and flexions of the paw are quantitated over the following 90 minute period. A biphasic response pattern is typically observed, with numerous responses observed during the period five minutes after injection (Phase 1) and a second phase (Phase 2), which occurs during the period about 10-60 minutes following injection. The mean number of flinches per minute is recorded as a function of time. Quantitation of responses during each phase can be accomplished by calculation of area under the curve of flinches/minute.)

Formalin-Induced Hyperalgesia:

The formalin induction model reflects several levels of processing of nociceptive information in the spinal cord. See, e.g., U.S. Pat. No. 6,166,085. Protracted sensory input generated by the noxious stimulus employed in this test (formalin in the paw) has been shown to induce an acute pain response phase (phase 1) followed by a second phase (phase 2). This second phase is thought to represent a state of facilitated processing evoked by the afferent input present during phase I and to involve release of at least two substances, glutamate and a tachykinin, based on pharmacological evidence. Injection of formalin into the paw evokes an initial burst of afferent input followed by a persistent low level discharge. This model results in a biphasic increase in the activity of dorsal horn wide dynamic range neurons, and a parallel biphasic appearance of flinching.

In normal non-stimulated lumbar spinal cord P-p38 MAPK levels are low. However, 5 minutes after formalin injection into the plantar surface of the hind paw, P-p38 MAPK immunoreactivity was detected in spinal homogenates by Western blotting. A 53% increase of the P-p38/p38 ratio was calculated based on densitometry measurements (0.26±0.078 and 0.40±0.083 for control versus formalin group). The functional significance of this activation is supported by the observation that intrathecal injection of either SD or SB resulted in a potent dose-dependent attenuation of the second phase of flinching behavior induced by the formalin injection into the paw (FIG. 2A, B). These results, showing a reversal of the hyperalgesia evoked by intrathecal sP, NMDA or the afferent input generated by a local irritant, indicate that p38 MAPK activation acutely contributes to altered spinal excitability, presumably though through downstream phosphorylation of target protein.

p38 MAPK Activation Relative to Altered Spinal Activity (Carageenan Mediated Hyperalgesia)

Hyperalgesia was induced in the rat's right hindpaw by intraplantar injection of carrageenan (2 mg in 0.1 ml of a 20% solution (weight/volume) in physiological saline). To assess the thermally evoked paw withdrawal time, a device modeled after that described by Hargreaves (1988) was used. See Hargreaves K, Dubner R, Brown F, Flores C, Joris J. A new and sensitive method for measuring thermal nociception in cutaneous hyperalgesia, Pain, 32, 77-88 (1988). The device consisted of a glass surface on top of which the rats were placed. The glass surface was heated by a focused projection bulb below the glass surface. The first sign of discomfort is usually expressed as an attempt to sit up and lick the forepaws by the experimental animal. This response indicates a threshold of pain under the predetermined conditions. Dancing and jumping about by an undrugged animal is an indicator of unbearable pain; whereas drugged animals more commonly withdraw the hind paws and keep them close to their abdomen. A timer was actuated with the light source and latency defined as the time required for the paw to be withdrawn from the glass surface. See Dirig D M, Isakson P C, and Yaksh T L. J Pharmacol Exp Ther., 285, 1031-8 (1998).

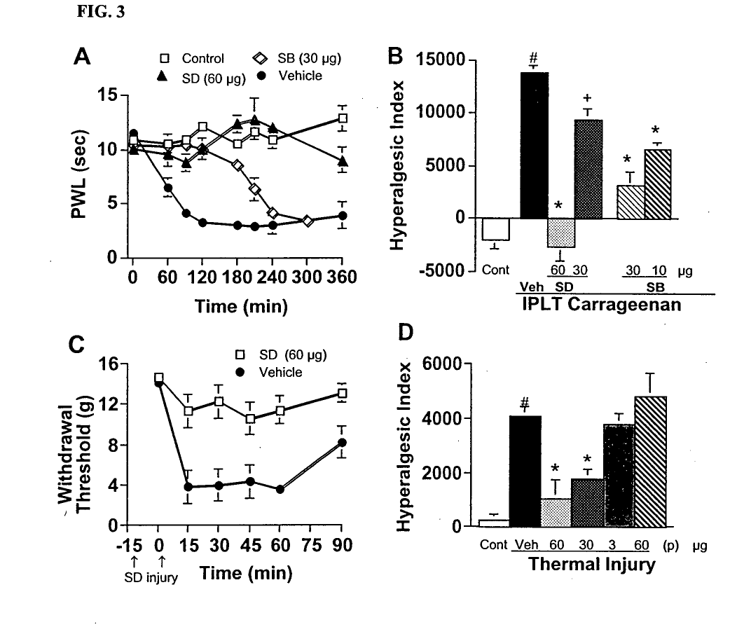

Pre-treatment with either IT SD or IT SB resulted in a potent dose-dependent suppression of carrageenan-induced thermal hyperalgesia (FIG. 3A, B). Importantly, this effect was observed only on the reduced latencies of the induced paw. There were no changes, even after the highest doses of either drug, in the response latency of the uninduced paw (p>0.5, data not shown).

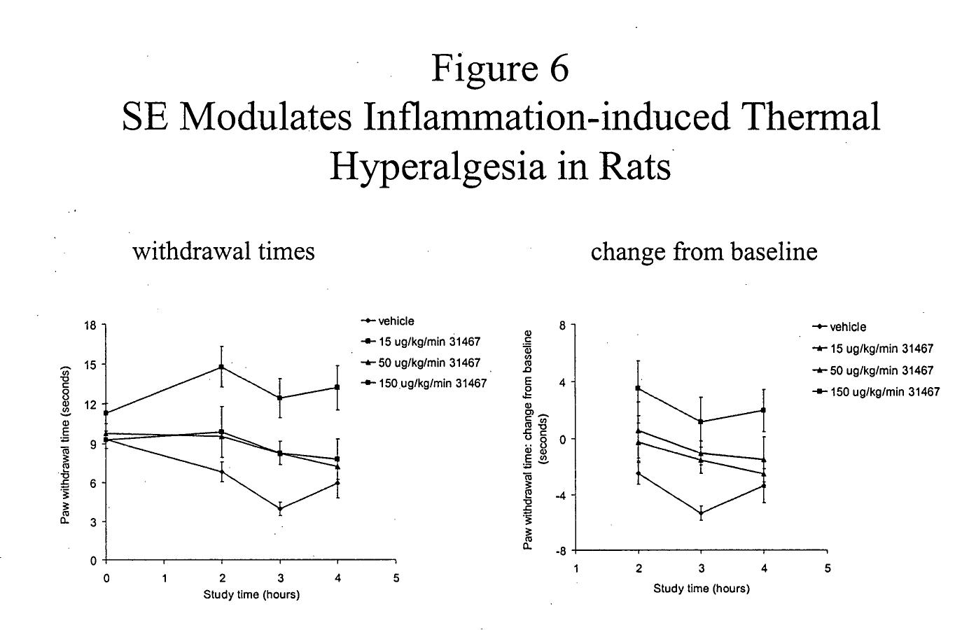

Attenuation of the carageenan induced hyperalgesia was also observed with the intravenous administration of SE, another p38 inhibitor. As shown in FIG. 6, responses similar to those with IT SD and SB were achieved. Dosages were varied and infer some correlation relative to observed response.

p38 MAPK Activation Relative to Thermal Injury Induced Allodynia

To determine if the effects of p38 MAPK inhibition were restricted to a thermal modality, we examined the effects of IT SD on tactile allodynia produced by a local unilateral thermal injury. Thermal injury was induced by placing the plantar surface of the right hindpaw on a hot plate (52.0+/−1 degree C.) for 45 sec. Thermal escape latencies and evoked paw-withdrawal responses were assessed using an under-glass thermal stimulus. See Dirig et al. Tactile allodynia was assessed by determining the threshold stimulus for evoking hindpaw withdrawal by calibrated filaments (15.14-0.41 g) delivered in an up-down paradigm. See S. R. Chaplan, F. W. Bach, J. W. Pogrel, J. M. Chung, and T. L. Yaksh, J Neurosci Methods 53, 55-63. (1994). As shown in FIGS. 3C and D, IT SD resulted in a dose dependent suppression of the tactile allodynia.

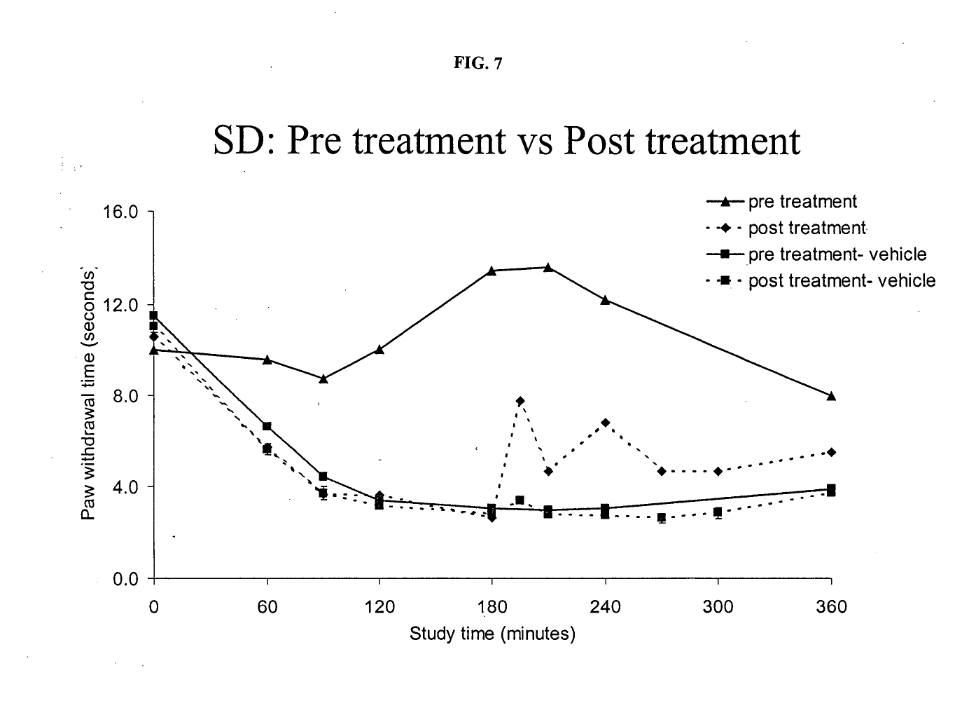

In a variation to this burn model, the effectiveness of IT SD was evaluated when it was administered pre-injury as opposed to post-injury. The results are presented in FIG. 7. In comparison to administration of IT SD post injury, pre-treatment yielded substantially higher levels of attenuation for tactile allodynia. This suggests that with IT (central) administration, pre-dosing with the inhibitors may be more effective than treatment after an injury has occurred.

p38 MAPK Regulation of Relevant Transcription Factors and Protein Expression.

To determine if spinal p38 MAPK is involved in regulation of transcription factors and protein expression associated with nociceptive stimulation an examination was done on the effects of p38 MAPK inhibition on activation of spinal cFOS, an immediate early gene reflective of neuronal activation, and upregulation of COX-2, an enzyme important to the injury induced facilitation at the spinal level. Examination was conducted as follows: Transverse spinal cord sections (20 μm) were cut and processed for immunohistochemistry using a FOS antibody (Calbiochem, 1:50000) as described by L. C. Yang, et al., Cell Mol Neurobiol 20, 351-65 (2000). For colocalization studies transverse spinal cord sections (10 μm) were cut and processed for confocal microscopy using polyclonal p38 and P-p38 antibody (Cell Signaling Technology), and monoclonal OX-42 (Biosource International, 1:100), Neu N (Chemicon, 1:1000), GFAP (Chemicon, 1:200) and APC (Oncogene, 1:500) antibodies.

Following intraplantar formalin, a pronounced increase was observed in the number of FOS immunoreactive neurons in the ipsilateral lamina 1-5 at the lumbar 2-6 level of the dorsal horn after 2 hours (FIG. 2C-E). Intrathecal injection of the selective p38 MAPK inhibitor SD 10 minutes prior to the formalin injection resulted in a decrease in the number of FOS positive neurons as compared to vehicle-treated controls. See FIG. 2F.

Upregulation of COX-2 after peripheral tissue injury has been demonstrated and suggested to play an important role in the evolution of the persistent hyperalgesia noted in chronic inflammatory states. Accordingly, the effect of p38 MAPK inhibition on the expression of COX-2 protein levels in the spinal cord was explored. As shown in FIG. 1D, IT sP results in a significant increase of COX-2 in dorsal horn 4 hours post injection. Prior inhibition of spinal p38 MAPK with IT SD, at a dose that blocked the thermal hyperalgesia, also prevented the subsequent upregulation of COX-2 protein otherwise noted following IT sP, while no changes was seen in the levels of COX-1. These results suggest that p38 MAPK inhibition blocks the down stream events initiated by NK-1 receptor activation.

Pre Versus Post Administration of p38 Inhibitor (Formalin and Thermal Paw Injury)

An examination was conducted on the effects of IT SD relative to flinching and tactile allodynia when an inhibitor was given 5 min after the paw formalin injection and the thermal paw injury, respectively. In these studies it was noted that, in contrast to pre-treatment, post-treatment did not result in a statistically significant antihyperalgesic activity (see FIGS. 2B and 3D). Also, post-treatment with IT SD did not prevent the IT sP-evoked increase of spinal COX-2 protein (FIG. 1D). The observation that SD was less effective when given after tissue injury than before suggests that p38 MAPK serves in mediating the initiation of down stream processes begun by the small afferent input.

Cell Populations Implicated by p38 MAPK and Spinal Nociceptive Processing

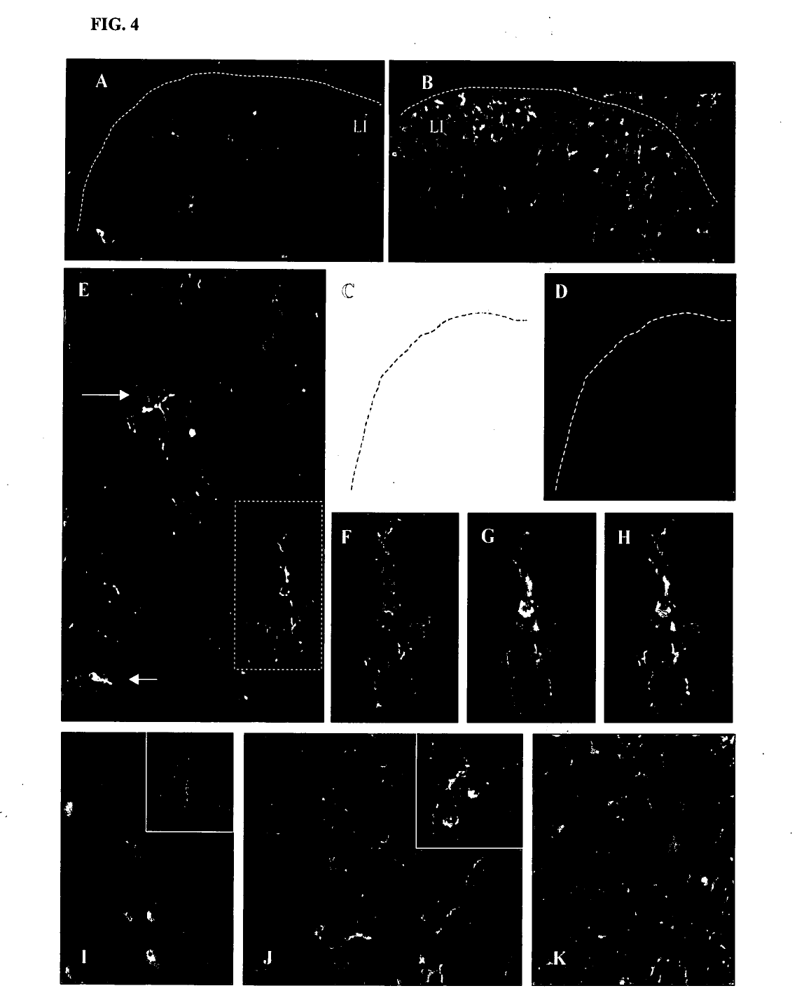

The potency and pervasive effects of p38 MAPK inhibition observed in the studies above, led to an evaluation relative to what cell populations p38 kinase is located. Rats received saline or IT sP and 10 minutes later spinal cords were harvested and fixed. Frozen transverse spinal cord sections taken from L3-L6 segments were prepared and stained with antibodies for activated p38 MAPK, microglia (OX-42), neurons (Neu N), astrocytes (GFAP) or oligodendrocytes (APC). In contrast to saline injected animals a significant increase in the number of p-p38 MAPK positive cells were detected after IT sP. P-p38 MAPK positive cells were localized predominantly in the superficial (I-II) and deep (VI-VII) dorsal laminae (FIG. 4A, B). Unexpectedly, confocal analysis revealed an exclusive co-localization with microglia (FIG. 4C-F). No P-p38 MAPK expression was detected in neurons, astrocytes or oligodendrocytes (FIG. 4G-I). In addition to the increased number of p-p38 MAPK positive microglial cells, these immunoreactive cells also displayed morphological signs of activation. Though not systematically quantified, examination of the histochemistry emphasized an increase in cell body size and processes. In these cells, varicosity-like profiles were observed in proximity of limiting membrane of neuronal N positive cells. Taken together these data suggest that activated microglial may represent a primary source of p38 and that microglia may accordingly play an important role in spinal nociceptive processing.

### Example 2

### Additional Studies Evaluating the Effects of p38 MAP Kinase Inhibitors on Mechanical and Thermal Hyperalgesia

The effects of p38 MAP kinase inhibitors on Mechanical and Thermal Hyperalgesia were evaluated as follows. Sprague Dawley Rats were evaluated in carageenan induced models whereby an intraplantar injection of carageenan was made into the left paw. At approximately 2 and 4 hour post carageenan, the left hind limb of each rat was assessed for the development of thermal hyperalgesia relative to a p38 MAP kinase inhibitor (SC), vehicle, indomethacin, and the vehicle for indomethacin. Indomethacin, a known nonsteroidal analgesic, was used as a control reference in this study. Assessment of mechanical and thermal hyperalgesia was accomplished through the use of a Randall Selitto Analgesiometer and the Hargreaves Plantar Device, respectively.

Development of Mechanical Hyperalgesia

Approximately 10 minutes after intraperitoneal dosing, or 30 minutes following oral indomethacin treatment, each animal was lightly anaesthetized (isoflurane in oxygen) and an intraplantar injection of carageenan was made into the left paw. Animals were allowed to recover from anesthesia.

Mechanical hyperalgesia development was observed in the vehicle treated group by the 4 hours post-carrageenan time point (105±13 g; P≦0.05), compared to pre dose (156±9 g). There was no mechanical hyperalgesic state seen in the vehicle treated group at 2 hours post-carrageenan.

Mechanical Hyperalgesia (Randall Selitto Test)

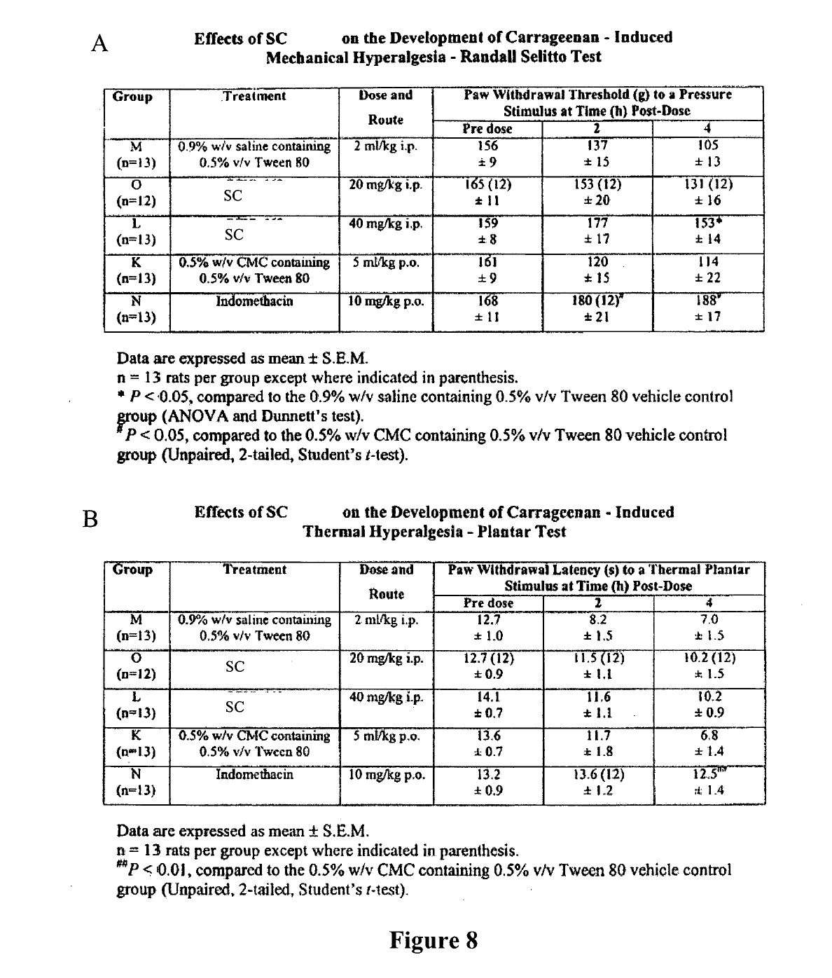

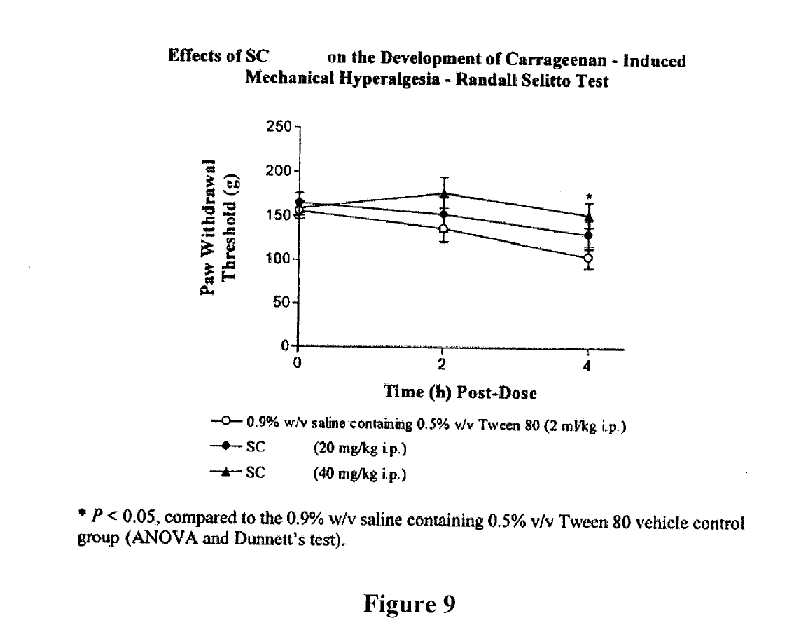

Mechanical hyperalgesia was tested using the Randall-Selittlo test. Randall, L. & Selitto, J., Arch Int Pharmacodyn (1957) 110:409-419. The carageenan model was established as set forth above. Approximately 30 minutes after intraplantar injection of carrageenan, administration of SC significantly attenuated the development of mechanical hyperalgesia at the 4 hour time point (153±14 g; P≦0.05) when compared to the vehicle treated group (105±13 g). In the same animals treated with SC, the paw withdrawal threshold increased from a pre dose threshold of 159±8 g to 177±17 g, 2 hours post-carrageenan. See FIGS. 8 and 9. In the same experiment, oral administration of indomethacin (10 mg/kg) exhibited a statistically significant increase in paw withdrawal threshold at both the 2 hours (180±21 g; P≦0.05) and the 4 hours time points (188±17 g; P±0.05), compared to the vehicle for indomethacin treated group (120±15 and 114±22 g, respectively).

Evaluation of Thermal Hyperalgesia (Plantar Test)

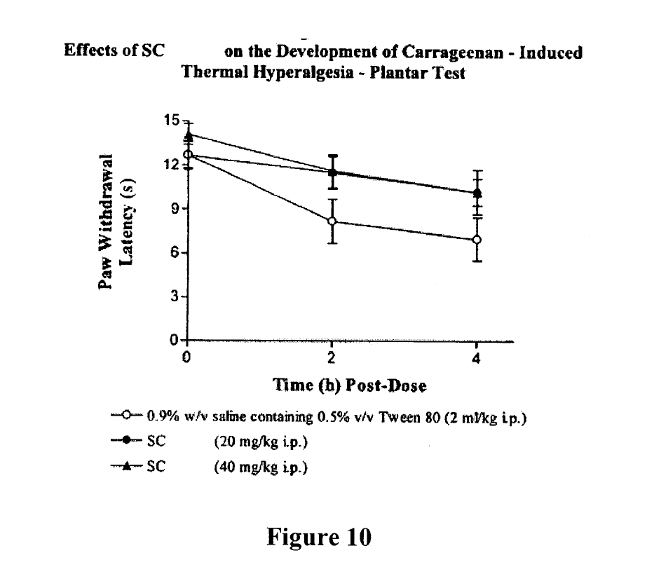

As set forth above in the carageenan model, the effect of SC was evaluated with respect to attenuation of the development of thermal hyperalgesia at either of the 2 and 4 hours post-carrageenan time points tested. As above with respect to mechanical hyperalgesia, observation of statistically significant thermal hyperalgesia development was made at both the 2 and 4 hour periods (8.2±1.5 s; P≦0.05 and 7.0±1.5 s; P≦0.01, respectively), compared to vehicle pre dose measurements (12.7±1.0 s). Relative to SC, there was an observed trend towards attenuation in thermal hyperalgesia observed at both dose levels tested at both the 2 hours time point (11.5±1.1 and 11.6±1.1 s, respectively), when compared to the vehicle group (8.2±1.5 s) and 4 hours time point (10.2±1.5 and 10.2±0.9 s, respectively), when compared to the vehicle group (7.0±1.5 s). See FIGS. 8 and 10.

The reference substance, indomethacin (10 mg/kg p.o.), significantly attenuated the development of thermal hyperalgesia at the 4 hours time point only (12.5±1.4 s; P≦0.01), when compared to the vehicle for indomethacin treated group (6.8±1.4 s). The withdrawal latency for indomethacin treated animals increased by 2 hours post-carrageenan, however, this was not significant.

From the outcome referenced above, the animals treated with intraperitoneal SC exhibited significant attenuation in the development of mechanical hyperalgesia at the 4 hour time point. At the 2 hours assessment, more than one dose of SC exhibited a trend towards attenuation in mechanical hyperalgesia. The effects of SC on thermal hyperalgesia development also indicated a trend in attenuation for both dose levels tested.

In summary, SC exhibited an ability to significantly attenuate the development of mechanical hyperalgesia. A trend towards attenuation in thermal hyperalgesia development was also observed at both dose levels tested. These results indicate that SC may possess selective antinociceptive properties.

As expected, the development of mechanical and thermal hyperalgesia associated with intraplantar injection of 0.6% w/v carrageenan lambda were significantly attenuated by prior administration of 10 mg/kg (p.o.) indomethacin. From this data, SC appears to possess antinociceptive properties.

### Example 3

### The Effect of Orally Administered p38 Inhibitor

As set forth in Example 2 above, Sprague Dawley rats were evaluated in the intraplantar carageenan model. The animals were administered the p38 MAP kinase inhibitor SA, vehicle, indomethacin, and the vehicle for indomethacin. After dosing, the animals were assessed for the development of mechanical hyperalgesia and thermal hyperalgesia using the Randall Selitto Analgesiometer and the Hargreaves Plantar Device, respectively.

Mechanical Hyperalgesia (Randall Selitto Test)

In the model, mechanical hyperalgesia development was observed in the vehicle treated group at the 4 hour post-carrageenan time point (121±15 g; P≦0.05), in comparison to the pre dose value (167±12 g).

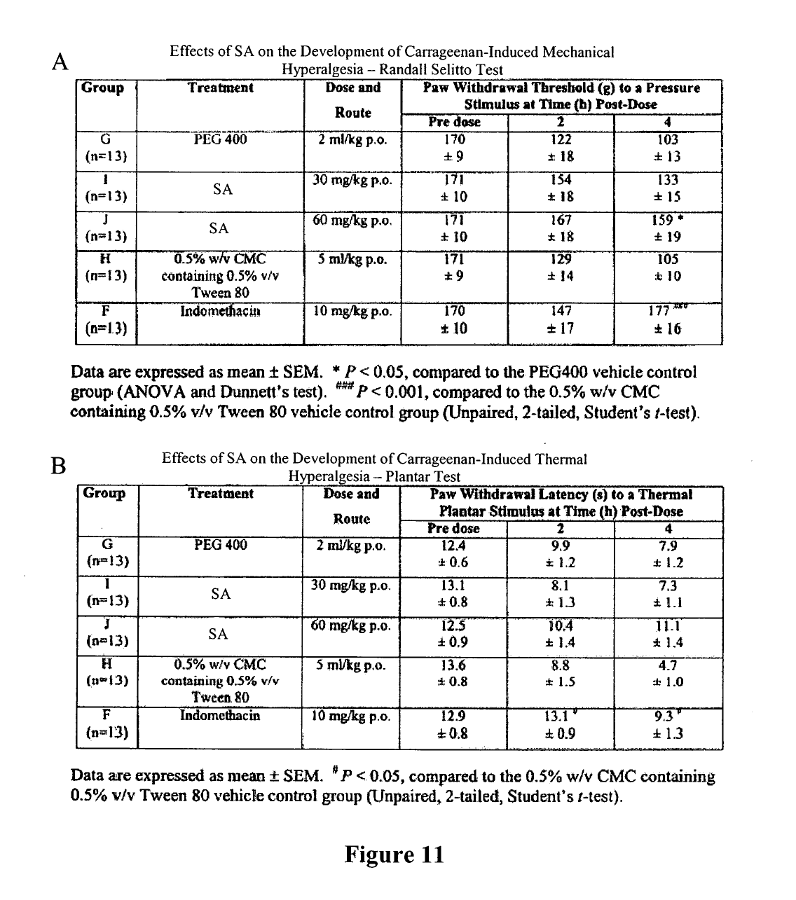

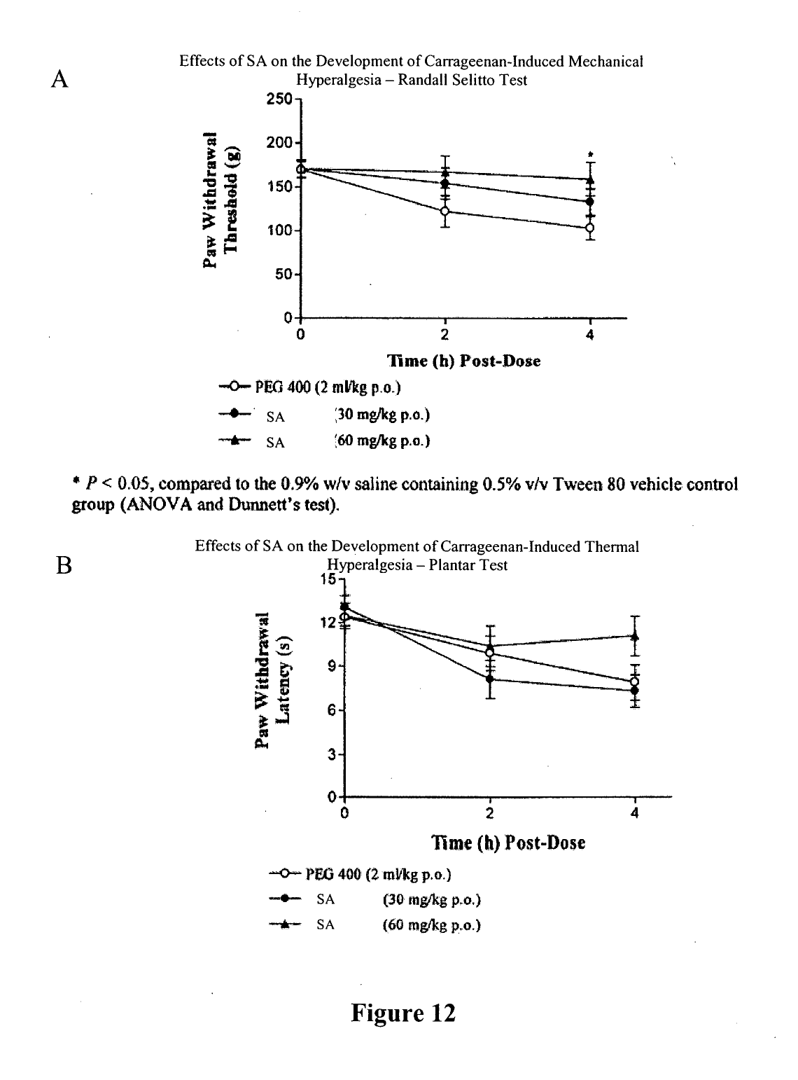

Substance A (SA) was administered orally 30 minutes prior to intraplantar injection of carrageenan. As shown in FIGS. 11 and 12, SA significantly attenuated the development of mechanical hyperalgesia at the 4 hours time point (159±19 g; P≦0.05) when compared to the vehicle treated group (103±13 g). Oral administration of indomethacin (10 mg/kg) significantly attenuated the development of mechanical hyperalgesia at the 4 hours time point (177±16 g; P≦0.001), compared to the vehicle for indomethacin treated group (105±10 g).

Thermal Hyperalgesia (Plantar Test)

In the model, thermal hyperalgesia development was statistically significant by the 4 hours observation period (7.9±1.2 s; P≦0.01), in comparison to the pre dose value (12.4±0.6 s).

Oral administration of SA prevented the development of thermal hyperalgesia as observed by the lack of a notable reduction in withdrawal latency at the 4 hours time point tested (11.1±1.4 s; P≦0.05), when compared to the vehicle group (7.9±1.2 s). At lower dose levels of SA, no significant attenuation was observed. The reference substance, indomethacin (10 mg/kg p.o.), significantly attenuated the development of thermal hyperalgesia at both the 2 and 4 hours time points (13.1±0.9 s; P≦0.05 and 9.3±1.3 s; P≦0.05, respectively), when compared to the vehicle for indomethacin treated group (8.8±1.5 and 4.7±1.0 s, respectively).

In summary, oral administration of SA significantly inhibited mechanical hyperalgesia. Attenuation in the development of thermal hyperalgesia was also observed in this study. These results indicate that SA possesses possible selective or non-selective antinociceptive properties.

Conclusion

Unexpectedly, the above studies suggest that p38 MAPK plays a pivotal role in the acute and persistent events affiliated with the transmission of pain initiated by tissue and other peripheral injuries. p38 MAPK seems to be an early component in the spinal cascade, linking the stimulus events and the down stream cellular processes. It is likely that p38 MAPK is also induced at the peripheral site of injury. p38 modulators are effective when administered intrathecally as well as peripherally, suggesting spinal as well as peripheral sites of action. Regardless of the mechanism, the administration of a p38 MAPK inhibitor in a therapeutically effective dosage prevents or treats pain in mammals.

### Example 4

### Treatment of Pain Associated with a Dental Procedure

A subject scheduled for a dental procedure, the filling of a cavity in a tooth, is administered approximately 40 mg/kg of the p38 MAP kinase inhibitor SF approximately 1 hours before the procedure is to begin. No other analgesics or anesthetics are administered. The dental procedure is performed and the subject experiences a reduced level of discomfort as compared to a subject having the same procedure in the absence of analgesics or anesthetics.

### Example 5

### Treatment of Pain Associated with a Dental Procedure

A subject scheduled for a dental procedure, the filling of a cavity in a tooth, is administered approximately 20 mg/kg of the p38 MAP kinase inhibitor SG approximately 1 hours before the procedure is to begin. No other analgesics or anesthetics are administered. The dental procedure is performed and the subject experiences a reduced level of discomfort as compared to a subject having the same procedure in the absence of analgesics or anesthetics.

### Example 6

### Treatment of Pain Associated with Athletic Injuries

A subject preparing for an athletic endeavor, the running of a long distance race, is administered approximately 50 mg/kg of SH approximately 30 minutes before the endeavor is to begin. No other analgesics or anesthetics are administered. The athlete participates in and completes the endeavor. The athlete experiences a reduced level of post-activity related discomfort as compared to a subject in a similar physical condition as the athlete how has participates in a similar athletic endeavor.

### Example 7

### Treatment of Pain Associated with Athletic Injuries

A subject preparing for an athletic endeavor, the running of a long distance race, is administered approximately 20 mg/kg of SI approximately 1 hours before the endeavor is to begin. No other analgesics or anesthetics are administered. The athlete participates in and completes the endeavor. The athlete experiences a reduced level of post-activity related discomfort as compared to a subject in a similar physical condition as the athlete how has participates in a similar athletic endeavor.

### Example 8

### Treatment of Pain Associated with Athletic Injuries

A subject preparing for an athletic endeavor, the running of a long distance race, is administered approximately 40 mg/kg of SM approximately 1 hours before the endeavor is to begin. No other analgesics or anesthetics are administered. The athlete participates in and completes the endeavor. The athlete experiences a reduced level of post-activity related discomfort as compared to a subject in a similar physical condition as the athlete how has participates in a similar athletic endeavor.

### Example 9

### Adjunct to Pre-Labor Anesthesia

A woman scheduled for a Cesarean section is prepared according to standard guidelines. A subarachnoid block is performed in the sitting position, following the administration of 1-2 liters of crystalloid solution. Skin infiltration with local anaesthetic is performed at the L2-3 or L3-L4 interspace. A spinal needle introducer is used to facilitate insertion of the needle into the patient. The needle is introduced into the epidural space and perforates the dura. The emergence of cerebrospinal fluid indicates proper placement of the needle. An opiod solution containing approximately 60 mg/kg of SL is administered and injected slowly of a ten to fifteen second time interval. The concentration of opiates in the solution is reduced because of the presence of the p38 MAP kinase inhibitor in the solution. The needle is then removed and resulting wound is dressed. The Cesarean section proceeds according to a standard protocol. The woman recovers more rapidly from the procedure because the reduced concentration of opiates in the anesthesia has a decreased inhibitory effect on her bowel function.

### Example 10

### Adjunct to Pre-Labor Anesthesia

A woman scheduled for a Cesarean section is prepared according to standard guidelines. A subarachnoid block is performed in the sitting position, following the administration of 1-2 liters of crystalloid solution. Skin infiltration with local anaesthetic is performed at the L2-3 or L3-L4 interspace. A spinal needle introducer is used to facilitate insertion of the needle into the patient. The needle is introduced into the epidural space and perforates the dura. The emergence of cerebrospinal fluid indicates proper placement of the needle. An opiod solution containing approximately 630 mg/kg of SJ is administered and injected slowly of a ten to fifteen second time interval. The concentration of opiates in the solution is reduced because of the presence of the p38 MAP kinase inhibitor in the solution. The needle is then removed and resulting wound is dressed. The Cesarean section proceeds according to a standard protocol. The woman recovers more rapidly from the procedure because the reduced concentration of opiates in the anesthesia has a decreased inhibitory effect on her bowel function.

### Example 11

### Adjunct to Pre-Labor Anesthesia

A woman scheduled for a Cesarean section is prepared according to standard guidelines. A subarachnoid block is performed in the sitting position, following the administration of 1-2 liters of crystalloid solution. Skin infiltration with local anaesthetic is performed at the L2-3 or L3-L4 interspace. A spinal needle introducer is used to facilitate insertion of the needle into the patient. The needle is introduced into the epidural space and perforates the dura. The emergence of cerebrospinal fluid indicates proper placement of the needle. An opiod solution containing approximately 50 mg/kg of SK is administered and injected slowly of a ten to fifteen second time interval. The concentration of opiates in the solution is reduced because of the presence of the p38 MAP kinase inhibitor in the solution. The needle is then removed and resulting wound is dressed. The Cesarean section proceeds according to a standard protocol. The woman recovers more rapidly from the procedure because the reduced concentration of opiates in the anesthesia has a decreased inhibitory effect on her bowel function.

Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A method to prevent or treat pain in a mammal in need thereof comprising administering an inhibitor of p38 kinase in a therapeutically effective amount to said mammal.

2. The method of claim 1, wherein said p38 MAP kinase inhibitor is selected from compounds of formula: 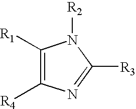
wherein
R₁ is a heteroaryl ring selected from 4-pyridyl, pyrimidinyl, quinolyl, isoquinolinyl, quinazolin-4-yl, 1-imidazolyl, 1-benzimidazolyl, 4-pyridazinyl, and a 1,2,4-triazin-5-yl ring, which heteroaryl ring is substituted one to three times with Y, N(R₁₀)C(O)R_{b}, a halo-substituted mono- or di-C₁₋₆ alkyl-substituted amino, or NHRₐ and which ring is further optionally substituted with C₁₋₄ alkyl, halogen, hydroxyl, optionally-substituted C₁₋₄ alkoxy, optionally-substituted C₁₋₄ alkylthio, optionally-substituted C₁₋₄ alkylsulfinyl, CH₂OR₁₂, amino, mono- and di-C₁₋₆ alkyl-substituted amino, NHRₐ, N(R₁₀)C(O)R_{b}, N(R₁₀)S(O)₂R_{d}, or an N-heterocyclyl ring which has from 5 to 7 members and optionally contains an additional heteroatom selected from oxygen, sulfur or NR₁₅;
Y is X₁—Rₐ;
X₁ is oxygen or sulfur;
Rₐ is C₁₋₆ alkyl, aryl, arylC₁₋₆ alkyl, heterocyclic, heterocyclylC₁₋₆ alkyl, heteroaryl, or heteroarylC₁₋₆ alkyl, wherein each of these moieties can be optionally substituted;
R_{b} is hydrogen, C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl;
R_{d} is C₁₋₆ alkyl, C₃₋₇ cycloalkyl, aryl, arylC₁₋₄ alkyl, heteroaryl, heteroarylC₁₋₄ alkyl, heterocyclyl, or heterocyclylC₁₋₄ alkyl;
R₃ is hydrogen;
R₄ is phenyl, naphth-1-yl, naphth-2-yl, or a heteroaryl, which is optionally substituted by one or two substituents, each of which is independently selected, and which, for a 4-phenyl, 4-naphth-1-yl, 5-naphth-2-yl or 6-naphth-2-yl substituent, is halogen, cyano, nitro, —C(Z)NR₇R₁₇, —C(Z)OR₁₆, —(CR₁₀R₂₀)—COR₁₂, —SR₅, —SOR₅, —OR₁₂, halo-substituted-C₁₋₄ alkyl, C₁₋₄ alkyl, -ZC(Z)R₁₂, —NR₁₀C(Z)R₁₆, or —(CR₁₀R₂₀)ᵥNR₁₀R₂₀ and which, for other positions of substitution, is halogen, cyano, —C(Z)NR₁₃R₁₄, —C(Z)OR_{f}, —(CR₁₀R₂₀)_{m″}COR_{f}, —S(O)ₘR_{f}, —OR_{f}, —OR₁₂, halo-substituted C₁₋₄ alkyl, C₁₋₄ alkyl, —(CR₁₀R₂₀)_{m″}NR₁₀C(Z)R_{f}, —NR₁₀S(O)_{m′}R₈, —NR₁₀S(O)_{m′}NR₇R₁₇, -ZC(Z)R_{f}, -ZC(Z)R₁₂, or —(CR₁₀R₂₀)_{m″}NR₁₃R₁₄;
R_{f} is heterocyclyl, heterocyclylC₁₋₁₀ alkyl or R₈;
Z is oxygen or sulfur;
v is 0, 1, or 2;
m is 0, 1, or 2;
m′ is 1 or 2;
m″ is 0, 1, 2, 3, 4, or 5;
R₂ is C₁₋₁₀ alkyl N₃, —(CR₁₀R₂₀)_{n′}OR₉, heterocylyl, heterocycylC₁₋₁₀ alkyl, C₁₋₁₀ alkyl, halo-substituted C₁₋₁₀ alkyl, C₂₋₁₀ alkenyl, C₂₋₁₀ alkynyl, C₃₋₇ cycloalkyl, C₃₋₇ cycloalkylC₁₋₁₀ alkyl, C₅₋₇ cycloalkenyl, C₅₋₇cycloalkenylC₁₋₁₀ alkyl, aryl, arylC₁₋₁₀ alkyl, heteroaryl, heteroarylC₁₋₁₀ alkyl, (CR₁₀R₂₀)ₙOR₁₁, (CR₁₀R₂₀)ₙS(O)ₘR₁₈, (CR₁₀R₂₀)ₙNHS(O)₂R₁₈, (CR₁₀R₂₀)ₙNR₁₃R₁₄, (CR₁₀R₂₀)ₙNO₂, (CR₁₀R₂₀)ₙCN, (CR₁₀R₂₀)_{n′}SO₂R₁₈, (CR₁₀R₂₀)ₙS(O)_{m′}NR₁₃R₁₄, (CR₁₀R₂₀)ₙC(Z)R₁₁, (CR₁₀R₂₀)ₙOC(Z)R₁₁, (CR₁₀R₂₀)ₙC(Z)OR₁₁, (CR₁₀R₂₀)ₙC(Z)NR₁₃R₁₄, (CR₁₀R₂₀)ₙC(Z)NR₁₁OR₉, (CR₁₀R₂₀)ₙNR₁₀C(Z)R₁₁, (CR₁₀R₂₀)ₙNR₁₀C(Z)NR₁₃R₁₄, (CR₁₀R₂₀)ₙN(OR₆)C(Z)NR₁₃R₁₄, (CR₁₀R₂₀)ₙN(OR₆)C(Z)R₁₁, (CR₁₀R₂₀)ₙC(═NOR₆)R₁₁, (CR₁₀R₂₀)ₙNR₁₀C(═NR₁₉)NR₁₃R₁₄, (CR₁₀R₂₀)ₙOC(Z)NR₁₃R₁₄, (CR₁₀R₂₀)ₙNR₁₀C(Z)NR₁₃R₁₄, (CR₁₀R₂₀)ₙNR₁₀C(Z)OR₁₀, 5-(R₁₈)-1,2,4-oxadiazol-3-yl or 4-(R₁₂)-5-(R₁₈R₁₉)-4,5-dihydro-1,2,4-oxadiazol-3-yl; wherein the aryl, arylalkyl, heteroaryl, heteroaryl alkyl, cycloalkyl, cycloalkyl alkyl, heterocyclic and heterocyclic alkyl groups can be optionally substituted;
n is an integer having a value of 1 to 10;
n′ is 0, or an integer having a value of 1 to 10;
R₅ is hydrogen, C₁₋₄ alkyl, C₂₋₄ alkenyl, C₂₋₄ alkynyl or NR₇R₁₇, excluding the moieties —SR₅ being —SNR₇R₁₇ and —S(O)R₅ being —SOH;
R₆ is hydrogen, a pharmaceutically-acceptable cation, C₁₋₁₀ alkyl, C₃₋₇ cycloalkyl, aryl, arylC₁₋₄ alkyl, heteroaryl, heteroarylC₁₋₁₀ alkyl, heterocyclyl, aroyl, or C₁₋₁₀ alkanoyl;
R₇ and R₁₇ are each independently selected from hydrogen or C₁₋₄ alkyl, or R₇ and R₁₇ together with the nitrogen to which they are attached form a heterocyclic ring of 5 to 7 members which ring optionally contains an additional heteroatom selected from oxygen, sulfur or NR₁₅;
R₈ is C₁₋₁₀ alkyl, halo-substituted C₁₋₁₀ alkyl, C₂₋₁₀ alkenyl, C₂₋₁₀ alkynyl, C₃₋₇ cycloalkyl, C₅₋₇ cycloalkenyl, aryl, arylC₁₋₁₀ alkyl, heteroaryl, heteroarylC₁₋₁₀ alkyl, (CR₁₀R₂₀)ₙOR₁₁, (CR₁₀R₂₀)ₙS(O)ₘR₁₈, (CR₁₀R₂₀)ₙNHS(O)₂R₁₈, or (CR₁₀R₂₀)ᵥNR₁₃R₁₄, wherein the aryl, arylalkyl, heteroaryl, and heteroaryl alkyl can be optionally substituted;
R₉ is hydrogen, —C(Z)R₁₁, optionally-substituted C₁₋₁₀ alkyl, S(O)₂R₁₈, optionally-substituted aryl or optionally-substituted arylC₁₋₄ alkyl;
R₁₀ and R₂₀ are each independently selected from hydrogen or C₁₋₄ alkyl;
R₁₁ is hydrogen, C₁₋₁₀ alkyl, C₃₋₇ cycloalkyl, heterocyclyl, heterocyclylC₁₋₁₀ alkyl, aryl, arylC₁₋₁₀ alkyl, heteroaryl or heteroarylC₁₋₁₀ alkyl;
R₁₂ is hydrogen or R₁₆;
R₁₃ and R₁₄ are each independently selected from hydrogen or optionally-substituted C₁₋₄ alkyl, optionally-substituted aryl or optionally-substituted arylC₁₋₄ alkyl, or together with the nitrogen to which they are attached form a heterocyclic ring of 5 to 7 members which ring optionally contains an additional heteroatom selected from oxygen, sulfur or NR₉;
R₁₅ is R₁₀ or C(Z)C₁₋₄ alkyl;
R₁₆ is C₁₋₄ alkyl, halo-substituted C₁₋₄ alkyl, or C₃₋₇ cycloalkyl;
R₁₈ is C₁₋₁₀ alkyl, C₃₋₇ cycloalkyl, heterocyclyl, aryl, arylC₁₋₁₀ alkyl, heterocyclyl, heterocyclylC₁₋₁₀ alkyl, heteroaryl or heteroarylC₁₋₁₀ alkyl; and
R₁₉ is hydrogen, cyano, C₁₋₄ alkyl, C₃₋₇ cycloalkyl or aryl;
or a pharmaceutically-acceptable salt thereof,
or wherein
R₁, Y, X₁, Rₐ, R_{b}, R_{d}, v, m, m′, m″, Z, n, n′, and R₅ are defined as above, and
R₂ is hydrogen, C₁₋₁₀ alkyl, halo-substituted C₁₋₁₀ alkyl, C₂₋₁₀ alkenyl, C₂₋₁₀ alkynyl, C₃₋₇ cycloalkyl, C₃₋₇ cycloalkylC₁₋₁₀ alkyl, C₅₋₇ cycloalkenyl, aryl, arylC₁₋₁₀ alkyl, heteroaryl, heteroarylC₁₋₁₀ alkyl, heterocyclyl, heterocyclylC₁₋₁₀ alkyl, (CR₁₀R₂₈)ₙOR₁₂, (CR₁₀R₂₈)_{n′}OR₁₃, (CR₁₀R₂₈)_{n′}S(O)ₘR₂₅, (CR₁₀R₂₈)ₙS(O)₂R₂₅, (CR₁₀R₂₈)_{n′}NHS(O)₂R₂₅, (CR₁₀R₂₈)_{n′}NR₈R₉, (CR₁₀R₂₈)_{n′}NO₂, (CR₁₀R₂₈)_{n′}CN, (CR₁₀R₂₈)_{n′}S(O)ₘNR₈R₉, (CR₁₀R₂₈)_{n′}C(Z)R₁₃, (CR₁₀R₂₈)_{n′}C(Z)OR₁₃, (CR₁₀R₂₈)_{n′}C(Z)NR₈R₉, (CR₁₀R₂₈)_{n′}C(Z)NR₁₃OR₁₂, (CR₁₀R₂₈)_{n′}NR₁₀C(Z)R₁₃, (CR₁₀R₂₈)_{n′}NR₁₀C(Z)NR₈R₉, (CR₁₀R₂₈)_{n′}N(OR₂₁)C(Z)NR₈R₉, (CR₁₀R₂₈)_{n′}N(OR₂₁)C(Z)R₁₃, (CR₁₀R₂₈)_{n′}C(═NOR₂₁)R₁₃, (CR₁₀R₂₈)_{n′}NR₁₀C(═NR₂₇)NR₈R₉, (CR₁₀R₂₈)_{n′}OC(Z)NR₈R₉, (CR₁₀R₂₈)_{n′}NR₁₀C(Z)OR₁₀, (CR₁₀R₂₈)_{n′}NR₁₀C(Z)OR₁₀, 5-(R₂₅)-1,2,4-oxadiazol-3-yl or 4-(R₁₂)-5-(R₁₈R₁₉)-4,5-dihydro-1,2,4-oxadiazol-3-yl; wherein the cycloalkyl, cycloalkyl alkyl, aryl, arylalkyl, heteroaryl, heteroarylalkyl, heterocyclyl, or heterocyclylalkyl moieties can be optionally substituted;
R₃ is hydrogen or Q-(Y)ₜ;
Q is an aryl or heteroaryl group;
t is 1, 2, or 3;
Y₁ is independently selected from hydrogen, C₁₋₅ alkyl, halo-substituted C₁₋₅ alkyl, halogen, or —(CR₁₀R₂₀)ₙY₂;
Y₂ is OR₈, NO₂, S(O)_{m″}R₁₁, SR₈, S(O)_{m″}OR₈, S(O)ₘNR₈R₉, NR₈R₉, O(CR₁₀R₂₀)_{n′}NR₈R₉, C(O)R₈, CO₂R₈, CO₂(CR₁₀R₂₀)_{n′}CONR₈R₉, ZC(O)R₈, CN, C(Z)NR₈R₉, NR₁₀C(Z)R₈, C(Z)NR₈OR₉, NR₁₀C(Z)NR₈R₉, NR₁₀S(O)_{m′}R₁₁, N(OR₂₁)C(Z)NR₈R₉, N(OR₂₁)C(Z)R₈, C(═NOR₂₁)R₈, NR₁₀C(═NR₁₅)SR₁₁, NR₁₀C(═NR₁₅)NR₈R₉, NR₁₀C(═CR₁₄R₂₄)SR₁₁, NR₁₀C(═CR₁₄R₂₄)NR₈R₉, NR₁₀C(O)C(O)NR₈R₉, NR₁₀C(O)C(O)OR₁₀, C(═NR₁₃)NR₈R₉, C(═NOR₁₃)NR₈R₉, C(═NR₁₃)ZR₁₁, OC(Z)NR₈R₉, NR₁₀S(O)_{m″}CF₃, NR₁₀C(Z)OR₁₀, 5-(R₁₈)-1,2,4-oxadiazol-3-yl or 4-(R₁₂)-5-(R₁₈R₁₉)-4,5-dihydro-1,2,4-oxadiazol-3-yl;
R₄ is phenyl, naphth-1-yl or naphth-2-yl which is optionally substituted by one or two substituents, each of which is independently selected, and which, for a 4-phenyl, 4-naphth-1-yl or 5-naphth-2-yl substituent, is halo, nitro, cyano, C(Z)NR₇R₁₇, C(Z)OR₂₃, (CR₁₀R₂₀)ᵥCOR₃₆, SR₅, SOR₅, OR₃₆, halo-substituted-C₁₋₄ alkyl, C₁₋₄ alkyl, ZC(Z)R₃₆, NR₁₀C(Z)R₂₃, or (CR₁₀R₂₀)ᵥNR₁₀R₂₀ and which, for other positions of substitution, is halo, nitro, cyano, C(Z)NR₁₆R₂₆, C(Z)OR₈, (CR₁₀R₂₀)_{m′}COR₈, S(O)ₘR₈, OR₈, halo-substituted-C₁₋₄ alkyl, C₁₋₄ alkyl, (CR₁₀R₂₀)_{m″}, NR₁₀C(Z)R₈, NR₁₀S(O)_{m′}R₁₁, NR₁₀S(O)_{m′}NR₇R₁₇, ZC(Z)R₈ or (CR₁₀R₂₀)_{m″}NR₁₆R₂₆;
R₇ and R₁₇ are each independently selected from hydrogen or C₁₋₄ alkyl, or R₇ and R₁₇ together with the nitrogen to which they are attached form a heterocyclic ring of 5 to 7 members, which ring optionally contains an additional heteroatom selected from oxygen, sulfur or NR₂₂;
R₈ is hydrogen, heterocyclyl, heterocyclylalkyl or R₁₁;
R₉ is hydrogen, C₁₋₁₀ alkyl, C₂₋₁₀ alkenyl, C₂₋₁₀ alkynyl, C₃₋₇ cycloalkyl, C₅₋₇ cycloalkenyl, aryl, arylalkyl, heteroaryl or heteroarylalkyl, or R₈ and R₉ can together with the nitrogen to which they are attached form a heterocyclic ring of 5 to 7 members, which ring optionally contains an additional heteroatom selected from oxygen, sulfur or NR₁₂;
R₁₀ and R₂₀ are each independently selected from hydrogen or C₁₋₄ alkyl;
R₁₁ is C₁₋₁₀ alkyl, halo-substituted C₁₋₁₀ alkyl, C₂₋₁₀ alkenyl, C₂₋₁₀ alkynyl, C₃₋₇ cycloalkyl, C₅₋₇ cycloalkenyl, aryl, arylalkyl, heteroaryl or heteroarylalkyl;
R₁₂ is hydrogen, —C(Z)R₁₃ or optionally-substituted C₁₋₄ alkyl, optionally-substituted aryl, optionally-substituted arylC₁₋₄ alkyl, or S(O)₂R₂₅;
R₁₃ is hydrogen, C₁₋₁₀ alkyl, C₃₋₇ cycloalkyl, heterocyclyl, heterocyclylC₁₋₁₀ alkyl, aryl, arylC₁₋₁₀ alkyl, heteroaryl or heteroaryl C₁₋₁₀ alkyl, wherein all of these moieties can be optionally substituted;
R₁₄ and R₂₄ are each independently selected from hydrogen, alkyl, nitro or cyano;
R₁₅ is hydrogen, cyano, C₁₋₄ alkyl, C₃₋₇ cycloalkyl or aryl;
R₁₆ and R₂₆ are each independently selected from hydrogen or optionally-substituted C₁₋₄ alkyl, optionally-substituted aryl or optionally-substituted arylC₁₋₄ alkyl, or together with the nitrogen to which they are attached form a heterocyclic ring of 5 to 7 members, which ring optionally contains an additional heteroatom selected from oxygen, sulfur or NR₁₂;
R₁₈ and R₁₉ are each independently selected from hydrogen, C₁₋₄ alkyl, substituted alkyl, optionally-substituted aryl, optionally-substituted arylalkyl, or together denote an oxygen or sulfur;
R₂₁ is hydrogen, a pharmaceutically-acceptable cation, C₁₋₁₀ alkyl, C₃₋₇ cycloalkyl, aryl, arylC₁₋₄ alkyl, heteroaryl, heteroarylalkyl, heterocyclyl, aroyl, or C₁₋₁₀ alkanoyl;
R₂₂ is R₁₀ or C(Z)-C₁₋₄ alkyl;
R₂₃ is C₁₋₄ alkyl, halo-substituted-C₁₋₄ alkyl, or C₃₋₅ cycloalkyl;
R₂₅ is C₁₋₁₀ alkyl, C₃₋₇ cycloalkyl, heterocyclyl, aryl, arylalkyl, heterocyclyl, heterocyclylC₁₋₁₀ alkyl, heteroaryl or heteroarylalkyl;
R₂₇ is hydrogen, cyano, C₁₋₄ alkyl, C₃₋₇ cycloalkyl, or aryl;
R₂₈ is hydrogen, C₁₋₆ alkyl, C₃₋₇ cycloalkyl, aryl, arylC₁₋₄ alkyl, heteroaryl, heteroarylC₁₋₄alkyl, heterocyclyl, or heterocyclylC₁₋₄ alkyl moiety, all of which can be optionally substituted; and
R₃₆ is hydrogen or R₂₃;
and a pharmaceutically acceptable salt thereof.

3. The method of claim 1, wherein said p38 MAP kinase inhibitor is selected from the compounds of the formula: 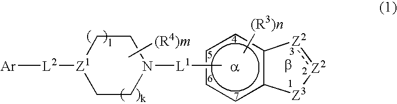
and the pharmaceutically acceptable salts thereof, or a pharmaceutical composition thereof, wherein [symbol]
represents a single or double bond;
one Z² is CA or CR⁸A and the other is CR¹, CR¹₂, NR⁶ or N wherein each R¹, R⁶ and R⁸ is independently hydrogen or noninterfering substituent;
A is —CO(X)ⱼY wherein Y is COR² or an isostere thereof and R² is hydrogen or a noninterfering substituent, X is a spacer of approximately 2-6 Å, and j is 0 or 1;
Z³ is NR⁷ or O;
each R³ is independently a noninterfering substituent;
n is 0-3;
each of L¹ and L² is a linker;
each R⁴ is independently a noninterfering substituent;
m is 0-4;
Z¹ is CR⁵ or N wherein R⁵ is hydrogen or a noninterfering substituent;
each of l and k is an integer from 0-2 wherein the sum of l and k is 0-3;
Ar is an aryl group substituted with 0-5 noninterfering substituents, wherein two noninterfering substituents can form a fused ring; and
the distance between the atom of Ar linked to L² and the center of the cc ring is approximately 4.5-24 Å.

4. The method of claim 1, wherein said p38 MAP kinase inhibitor is selected from the compounds of the formula: 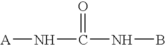
wherein A is 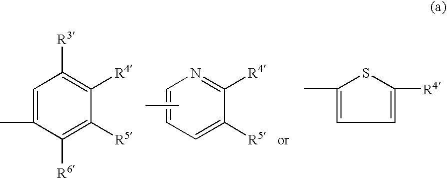
wherein
R^{3′}, R^{4′}, R^{5′} are each independently HOURS, C₁₋₁₀-alkyl, optionally substituted by halogen up to perhalo, C₁₋₁₀ alkoxy, optionally substituted by halogen, up to perhaloalkoxy, halogen; NO₂ or NH₂;
R^{6′} is HOURS, C₁₋₁₀-alkyl, C₁₋₁₀ alkoxy, —NHCOR¹; —NR¹COR¹; NO₂; 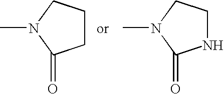
one of R^{4′}, R^{5′}, or R^{6′} can be —X—Y; or
2 adjacent R^{4′}-R^{6′} can together be an aryl or heteroaryl ring with 5-12 atoms, optionally substituted by C₁₋₁₀-alkyl, C₁₋₁₀ alkoxy, C₃₋₁₀ cycloalkyl, C₂₋₁₀ alkenyl, C₁₋₁₀ alkanoyl, C₆₋₁₂ aryl, C₅₋₁₂ heteroaryl or C₆₋₁₂ arakyl;
R₁ is C₁₋₁₀-alkyl optionally substituted by halogen, up to perhalo;
X is —CH₂—, —S—, —N(CH₃)—, —NHC(O)—, —CH₂—S—, —S—CH₂—, —C(O)—, or —O—;
X is additionally a single bond where Y is pyridyl;
Y is phenyl, pyridyl, naphthyl, pyridone, pyrazine, benzodioxane, benzopyridine, pyrimidine or benzothiazole, each optionally substituted by
C₁₋₁₀-alkyl, C₁₋₁₀-alkoxy, halogen, OH, —SCH₃ or NO₂ or, where Y is phenyl, by 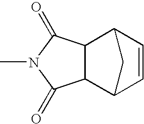
and a pharmaceutically-acceptable salt thereof;
or 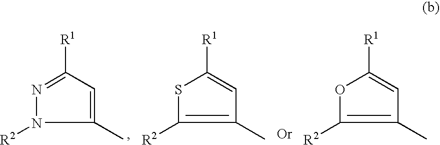
wherein
R¹ is selected from the group consisting of C₃-C₁₀ alkyl, C₃-C₁₀ cycloalkyl, up to per-halo substituted C₁-C₁₀ alkyl and up to per-halosubstituted C₃-C₁₀ cycloalkyl; and
R² is C₆-C₁₄ aryl, C₃-C₁₄ heteroaryl, substituted C₆-C₁₄ aryl or substituted C₃-C₁₄ heteroaryl;
wherein if R² is a substituted group, it is preferably substituted by one or more substituents independently selected from the group consisting of halogen, up to per-halosubstitution, and Vₙ, where n=0-3 and each V is independently selected from the group consisting of —CN, —OC(O)NR⁵R^{5′}, —CO₂R⁵, —C(O)NR⁵R^{5′}, —OR⁵, —SR⁵, —NR⁵R^{5′}, —C(O)R⁵, —NR⁵C(O)OR^{5′}, —SO₂R⁵—SOR⁵, —NR⁵C(O)R^{5′}, —NO₂, C₁-C₁₀ alkyl, C₃-C₁₀ cycloalkyl, C₆-C₁₄ aryl, C₃-C₁₃ heteroaryl, C₇-C₂₄ alkaryl, C₄-C₂₄ alkheteroaryl, substituted C₁-C₁₀ alkyl, substituted C₃-C₁₀ cycloalkyl, substituted C₆-C₁₄ aryl, substituted C₃-C₁₃ heteroaryl, substituted C₇-C₂₄ alkaryl and substituted C₄-C₂₄ alkheteroaryl;
wherein if V is a substituted group, it is substituted by one or more substituents independently selected from the group consisting of halogen, up to per-halosubstitution, —CN, —CO₂R⁵, —C(O)R⁵, —C(O)NR⁵R^{5′}, —NR⁵R^{5′}, —OR⁵, —SR⁵, NR⁵C(O)R^{5′}, —NR⁵C(O)OR^{5′} and —NO₂; and
R⁵ and R^{5′} are independently selected form the group consisting of HOURS, C₁-C₁₀ alkyl, C₃-C₁₀ cycloalkyl, C₆-C₁₄aryl, C₃-C₁₃ heteroaryl, C₇-C₂₄ alkaryl, C₄-C₂₃ alkheteroaryl, up to per-halosubstituted C₁-C₁₀ alkyl, up to per-halosubstituted C₃-C₁₀ cycloalkyl, up to per-halosubstituted C₆-C₁₄ aryl and up to per-halosubstituted C₃-C₁₃ heteroaryl;
and a pharmaceutically-acceptable salt thereof;
or
(c) a substituted moiety of up to 40 carbon atoms of the formula: -L-(M-L¹)_{q}, where L is a 5- or 6-membered cyclic structure bound directly to D, L¹, comprises a substituted cyclic moiety having at least 5 members, M is a bridging group having at least one atom, q is an integer of from 1-3; and each cyclic structure of L and L¹ contains 0-4 members of the group consisting of nitrogen, oxygen and sulfur;
L¹ is substituted by at least one substituent selected from the group consisting of —SO₂Rₓ, —C(O)Rₓ and —C(NR_{y})R_{z};
R_{y} is hydrogen or a carbon-based moiety of up to 24 carbon atoms optionally containing heteroatoms selected from N, S and O and optionally halosubstituted, up to perhalo;
R_{z} is hydrogen or a carbon-based moiety of up to 30 carbon atoms optionally containing heteroatoms selected from N, S and O and optionally substituted by halogen, hydroxy and carbon-based substituents of up to 24 carbon atoms, which optionally contain heteroatoms selected from N, S and O and are optionally substituted by halogen; and
Rₓ is R_{z}, or NRₐR_{b} where Rₐ and R_{b} are
i) independently hydrogen,
a carbon-based moiety of up to 30 carbon atoms optionally containing heteroatoms selected from N, S and O and optionally substituted by halogen, hydroxy and carbon-based substituents of up to 24 carbon atoms, which optionally contain heteroatoms selected from N, S and O and are optionally substituted by halogen, or
—OSi(R_{f})₃ where R_{f} is hydrogen or a carbon-based moiety of up to 24 carbon atoms optionally containing heteroatoms selected from N, S and O and optionally substituted by halogen, hydroxy and carbon-based substituents of up to 24 carbon atoms, which optionally contain heteroatoms selected from N, S and O and are optionally substituted by halogen; or
ii) Rₐ and R_{b} together form a 5-7 member heterocyclic structure of 1-3 heteroatoms selected from N, S and O, or a substituted 5-7 member heterocyclic structure of 1-3 heteroatoms selected from N, S and O, substituted by halogen, hydroxy or carbon-based substituents of up to 24 carbon atoms, which optionally contain heteroatoms selected from N, S and O and are optionally substituted by halogen; or
iii) one of Rₐ or R_{b} is —C(O)—, a C₁-C₅ divalent alkylene group or a substituted C₁-C₅ divalent alkylene group bound to the moiety L to form a cyclic structure with at least 5 members, wherein the substituents of the substituted C₁-C₅ divalent alkylene group are selected from the group consisting of halogen, hydroxy, and carbon-based substituents of up to 24 carbon atoms, which optionally contain heteroatoms selected from N, S and O and are optionally substituted by halogen;
and a pharmaceutically-acceptable salt thereof; and
B is an unsubstituted or substituted, up to tricyclic, aryl or heteroaryl moiety with up to 30 carbon atoms with at least one 5- or 6-membered aromatic structure containing 0-4 members of the group consisting of nitrogen, oxygen and sulfur;
wherein if B is substituted, it is substituted by one or more substituents selected from the group consisting of halogen, up to per-halo, and Wₙ, wherein
n is 0-3 and each W is independently selected from the group consisting of
—CN, —CO₂R⁷, —C(O)NR⁷R⁷, —C(O)R⁷, —NO₂, —OR⁷, —SR⁷, —NR⁷R⁷,
—NR⁷C(O)OR⁷, —NR⁷C(O)R⁷, C₁-C₁₀ alkyl, C₂₋₁₀-alkenyl, C₁₋₁₀-alkoxy, C₃-C₁₀ cycloalkyl, C₆-C₁₄ aryl, C₇-C₂₄ alkaryl, C₃-C₁₃ heteroaryl, C₄-C₂₃ alkheteroaryl, substituted C₁-C₁₀ alkyl, substituted C₂₋₁₀-alkenyl, substituted C₁₋₁₀-alkoxy, substituted C₃-C₁₀ cycloalkyl, substituted C₄-C₂₃ alkheteroaryl and -Q-Ar;
wherein if W is a substituted group, it is substituted by one or more substituents independently selected from the group consisting of —CN, —CO₂R⁷,
—C(O)NR⁷R⁷, —C(O)R⁷, —NO₂, —OR⁷, —SR⁷, —NR⁷R⁷, —NR⁷C(O)OR⁷,
—NR⁷C(O)R⁷ and halogen up to per-halo;
wherein each R⁷ is independently selected from HOURS, C₁-C₁₀ alkyl, C₂₋₁₀-alkenyl, C₃-C₁₀ cycloalkyl, C₆-C₁₄ aryl, C₃-C₁₃ heteroaryl, C₇-C₂₄ alkaryl, C₄-C₂₃ alkheteroaryl, up to per-halosubstituted C₁-C₁₀ alkyl, up to per-halosubstituted C₂₋₁₀-alkenyl, up to per-halosubstituted C₃-C₁₀ cycloalkyl, up to per-halosubstituted C₆-C₁₄ aryl and up to per-halosubstituted C₃-C₁₃ heteroaryl;
wherein Q is —O—, —S—, —N(R)⁷, —(CH₂)—ₘ, —C(O)—, —CH(OH)—, —NR⁷C(O)NR⁷R⁷—, —NR⁷C(O)—, —C(O)NR⁷—, —(CH₂)ₘO—, —(CH₂)ₘS—, —(CH₂)ₘN(R⁷)—, —O(CH₂)ₘ—, —CHX^{a}, —CX^{a}₂—, —S—(CH₂)ₘ— and —N(R⁷)(CH₂)ₘ—, where m=1-3, and X^{a} is halogen; and
Ar is a 5-10 member aromatic structure containing 0-4 members of the group consisting of nitrogen, oxygen and sulfur, which is unsubstituted or substituted by halogen up to per-halosubstitution and optionally substituted by Zₙ₁, wherein n1 is 0 to 3 and each Z substituent is independently selected from the group consisting of —CN, —CO₂R⁷, —C(O)NR⁷R⁷, —C(O)—NR⁷, —NO₂, —OR⁷, —SR⁷, —NR⁷R⁷, —NR⁷C(O)OR⁷, —C(O)R⁷, —NR⁷C(O)R⁷, C₁-C₁₀ alkyl, C₃-C₁₀ cycloalkyl, C₆-C₁₄ aryl, C₃-C₁₃ heteroaryl, C₇-C₂₄ alkaryl, C₄-C₂₃ alkheteroaryl, substituted C₁-C₁₀ alkyl, substituted C₃-C₁₀cycloalkyl, substituted C₇-C₂₄ alkaryl and substituted C₄-C₂₃ alkheteroaryl; wherein the one or more substituents of Z are independently selected from the group consisting of —CN, —CO₂R⁷, —C(O)NR⁷R⁷, —OR⁷, —SR⁷, —NO₂, —NR⁷R⁷, —NR⁷C(O)R⁷ and —NR⁷C(O)OR⁷;
and a pharmaceutically-acceptable salt thereof.

5. The method of claim 1 wherein said inhibitor is an inhibitor of p38α kinase.

6. The method of claim 1 wherein said inhibitor exhibits an IC₅₀ value for p38α kinase that is at least ten fold less than the IC₅₀ value said inhibitor exhibits relative to other isoforms of p38 MAP kinase.

7. A method for preventing a facilitative state for sensation of pain in a mammal comprising administering an inhibitor of p38 kinase in a therapeutically effective amount to said mammal.

8. The method of claim 7 wherein said facilitative state comprises hyperalgesia.

9. The method of claim 7 wherein said facilitative state comprises allodynia.

10. A method to prevent or treat pain in a mammal in need thereof comprising administering an inhibitor of p38 kinase in combination with an agent that inhibits pain and/or reduces inflammation in therapeutically effective amounts to said mammal.

11. A method to prevent pain in a mammal in need thereof comprising administering to said mammal an inhibitor of p38 kinase prior to a nociceptive event in a therapeutically effective amount.

12. A method to prevent pain in a mammal in need thereof comprising administering an inhibitor of p38 kinase in combination with an agent that inhibits pain and/or reduces inflammation in a therapeutically effective amount to said mammal.

13. A method of identifying a compound for preventing or treating pain in a mammal in need thereof, which comprises assaying candidate compounds for inhibition of p38 kinase activity, and identifying a compound that inhibits p38 kinase in a mammalian cell as indicative of a compound that alleviates or inhibits pain.

14. A method to prevent or treat pain in a mammal in need thereof comprising administering a compound identified by the method of claim 13 to said mammal.
